(19) United States
(12) Patent Application Publication
(10) Pub. No.: US 2024/0217961 A1
(43) Pub. Date: Jul. 4, 2024

(54) (S)-Mepazine Salt Forms, Process of Preparing, and Formulations Thereof

(71) Applicant: MONOPTEROS THERAPEUTICS, INC., Chestnut Hill, MA (US)

(72) Inventors: Peter Keller, Chestnut Hill, MA (US); Jon Lawson, Chestnut Hill, MA (US); Sami Karaborni, Chestnut Hill, MA (US); Qianwei Liu, Chestnut Hill, MA (US); Siyi Jiang, Chestnut Hill, MA (US)

(21) Appl. No.: 18/279,405

(22) PCT Filed: Mar. 4, 2022
(86) PCT No.: PCT/US22/18823

§ 371 Date: Aug. 30, 2023

Related U.S. Application Data
(60) Provisional application No. 63/157,314, filed on Mar. 5, 2021.

(51) Int. Cl. C07D 417/06; A61K 9/20; A61K 31/5415

(52) U.S. Cl. CPC C07D 417/06; A61K 9/2009; A61K 9/2018; A61K 9/2054; A61K 31/5415

(57) ABSTRACT

Provided herein are salt and crystalline forms of (S)-mepazine, formulations of the same, uses of the same, and processes of preparing (S)-mepazine.

### BACKGROUND

The compound (S)-10-(1-methylpiperidin-3-yl)methyl)-10H-phenothiazine (“(S)-mepazine”) is useful as an inhibitor of paracaspase, in particular, an inhibitor of MALT1 and thus useful in treating disorders and diseases in the development of which dysregulation of the activity of the paracaspase (e.g., MALT1) plays a role:

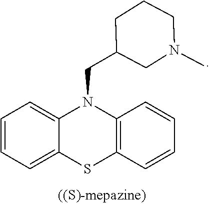

Research has shown that paracaspase (e.g., MALT1) inhibitors can be useful in the treatment of cancers. Exemplary cancers include carcinoma, a melanoma, a sarcoma, a myeloma, a leukemia, or a lymphoma. Exemplary cancers further include melanoma, colon cancer ovarian cancer, prostate cancer or cervical cancer. In addition, research has shown that paracaspase (e.g., MALT1) inhibitors can be useful in the treatment of paracaspase-dependent immune disease, such as allergic inflammation or an autoimmune disease. Exemplary paracaspase-dependent immune diseases include multiple sclerosis.

There is a need for various new salt and crystalline forms of (S)-mepazine with desirable chemical and physical properties, formulations of the same, processes of preparing (S)-mepazine and uses of the same.

### SUMMARY

Provided herein are salts having a structure of

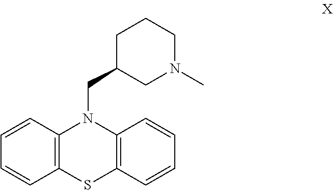 [SMILES: CN1CCC[C@H](CN2c3ccccc3Sc3ccccc32)C1]

wherein X comprises a conjugate base of an organic diacid. In embodiments, X is succinate, fumarate, hemi-fumarate, tartrate, malate, glutamate, or adipate.

Also provided herein are processes for synthesizing (S)-mepazine, or a salt thereof:

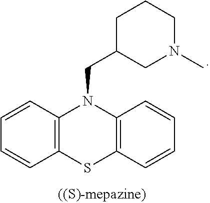

comprising: (a) admixing compound (J), a base, and a leaving group reagent in a solvent to form compound (K):

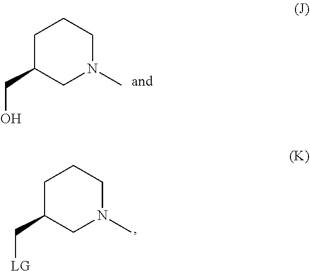

wherein LG is a leaving group; (b) forming a hydrochloride salt of compound (K); and (c) admixing the hydrochloride salt of compound (K) and phenothiazine in a solvent to form (S)-mepazine.

Also provided herein are pharmaceutical formulations comprising (S)-mepazine or a pharmaceutically acceptable salt thereof, and suitable excipients, in the form of a tablet. In embodiments, the tablet is an immediate release tablet.

Also provided herein are methods of treating a subject suffering from cancer, comprising administering to the subject a therapeutically effective amount of the salt as disclosed herein or the pharmaceutical formulation as disclosed herein.

Also provided herein are methods of treating a subject suffering from an autoimmune disease, comprising administering to the subject a therapeutically effective amount of the salt as disclosed herein or the pharmaceutical formulation as disclosed herein.

### BRIEF DESCRIPTION OF FIGURES

FIG. 1 depicts an X-ray powder diffraction (“XRPD”) pattern of the (S)-mepazine succinate crystalline salt form.

FIG. 2 depicts a differential scanning calorimetry (“DSC”) thermograph and a thermogravimetric analysis (“TGA”) trace of the (S)-mepazine succinate crystalline salt form.

FIG. 3 depicts an X-ray powder diffraction (“XRPD”) pattern of the (S)-mepazine fumarate salt crystalline form.

FIG. 4 depicts a differential scanning calorimetry (“DSC”) thermograph and a thermogravimetric analysis (“TGA”) trace of the (S)-mepazine fumarate salt crystalline form.

FIG. 5 depicts an X-ray powder diffraction (“XRPD”) pattern of the (S)-mepazine hemi-fumarate salt crystalline form I.

FIG. 6 depicts a differential scanning calorimetry (“DSC”) thermograph and a thermogravimetric analysis (“TGA”) trace of the (S)-mepazine hemi-fumarate salt crystalline form I.

FIG. 7 depicts an X-ray powder diffraction (“XRPD”) pattern of the (S)-mepazine hemi-fumarate salt crystalline form II.

FIG. 8 depicts a differential scanning calorimetry (“DSC”) thermograph and a thermogravimetric analysis (“TGA”) trace of the (S)-mepazine hemi-fumarate salt crystalline form II.

FIG. 9 depicts an XRPD pattern of the (S)-mepazine tartrate salt crystalline form I.

FIG. 10 depicts a differential scanning calorimetry (“DSC”) thermograph and a thermogravimetric analysis (“TGA”) trace of the (S)-mepazine tartrate salt crystalline form I.

FIG. 11 depicts an XRPD pattern of the (S)-mepazine tartrate salt crystalline form II.

FIG. 12 depicts a differential scanning calorimetry (“DSC”) thermograph and a thermogravimetric analysis (“TGA”) trace of the (S)-mepazine tartrate salt crystalline form II.

FIG. 13 depicts an XRPD pattern of the (S)-mepazine malate salt crystalline form.

FIG. 14 depicts a differential scanning calorimetry (“DSC”) thermograph and a thermogravimetric analysis (“TGA”) trace of the (S)-mepazine malate salt crystalline form.

FIG. 15 depicts an XRPD pattern of the (S)-mepazine glutamate salt crystalline form.

FIG. 16 depicts a differential scanning calorimetry (“DSC”) thermograph and a thermogravimetric analysis (“TGA”) trace of the (S)-mepazine glutamate salt crystalline form.

FIG. 17 depicts an XRPD pattern of the (S)-mepazine adipate salt crystalline form.

FIG. 18 depicts a differential scanning calorimetry (“DSC”) thermograph and a thermogravimetric analysis (“TGA”) trace of the (S)-mepazine adipate salt crystalline form.

FIG. 19 depicts an XRPD pattern of the crystalline (S)-mepazine free form.

FIG. 20 depicts a differential scanning calorimetry (“DSC”) thermograph and a thermogravimetric analysis (“TGA”) trace of the crystalline (S)-mepazine free form.

FIG. 21 depicts an XRPD pattern of the (S)-mepazine hydrochloride salt crystalline form.

FIG. 22 depicts a differential scanning calorimetry (“DSC”) thermograph and a thermogravimetric analysis (“TGA”) trace of the (S)-mepazine hydrochloride salt crystalline form.

### DETAILED DESCRIPTION

The present disclosure provides polymorphs and salts of (S)-10-((1-methylpiperidin-3-yl)methyl)-10H-phenothiazine, termed “(S)-mepazine” herein, and having a structure of:

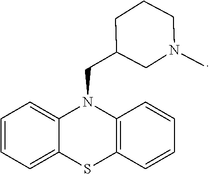

Embodiments of the salt forms of (S)-mepazine can be characterized by one or more of the parameters described in further detail below.

### Organic Diacid Salt Crystalline Forms of (S)-Mepazine

Provided herein are salt forms of (S)-mepazine and an organic diacid. In the crystalline forms of (S)-mepazine the counter-ion is a conjugate base of an organic diacid. Throughout, organic diacid used herein refers to the acid or conjugate base, unless specified otherwise. The, organic diacid of the disclosed salt forms is a C₁-C₁₀ organic diacid and comprises two carboxylic acid functional groups. In embodiments, the organic diacid can be a C₄-C₆ organic diacid. In embodiments, the organic diacid can be a polyol, i.e., comprise two or more (e.g., 2, 3, or 4) hydroxyl groups. Contemplated organic diacids include, but are not limited to, succinic acid, fumaric acid, tartaric acid, malic acid, glutamic acid, and adipic acid. Unless otherwise specified, the diacid salt is as present in a 0.9 to 1.1 molar ratio, e.g., 1 to 1 molar ratio, with the (S)-mepazine. In cases where the diacid is fumaric acid, the salt can be as a fumarate salt or as a hemi-fumarate salt.

In various cases, the organic diacid salt of (S)-mepazine is crystalline.

### Succinate Crystalline Salt Form

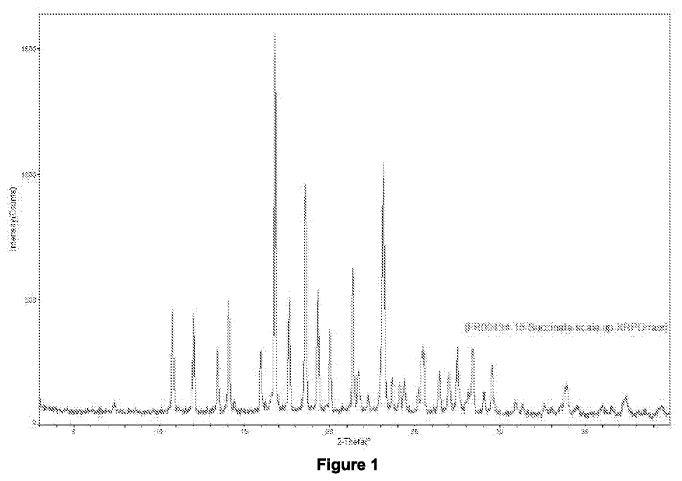

Provided herein is a succinate crystalline salt form of (S)-mepazine. The succinate crystalline salt form of (S)-mepazine can be characterized by an X-ray powder diffraction pattern, obtained as set forth in the Examples, having peaks at about 12.0, 16.8, and 18.6±0.2° 2θ using Cu Kα radiation. The succinate crystalline salt form of (S)-mepazine optionally can be further characterized by an X-ray powder diffraction pattern having additional peaks at about ±0.2° 2θ using Cu Kα radiation. The succinate crystalline salt form of (S)-mepazine optionally can be further characterized by an X-ray powder diffraction pattern having additional peaks at about 10.8, 16.0, 17.6, 19.3, and 23.2±0.2° 2θ using Cu Kα radiation. The succinate crystalline salt form of (S)-mepazine optionally can be further characterized by an X-ray powder diffraction pattern having additional peaks at about 3.4, 4.1, 13.5, 14.1, 20.0, 21.4, 21.7, 25.5, 27.0, 27.5, and 30.9±0.2° 2θ using Cu Kα radiation. The succinate crystalline salt form of (S)-mepazine optionally can be further characterized by an X-ray powder diffraction pattern having additional peaks at about 14.4, 23.7, 24.1, 24.4, 25.2, 28.1, 28.4, 29.1, 29.6, 32.6, and 33.9±0.2° 2θ using Cu Kα radiation. The succinate crystalline salt form of (S)-mepazine optionally can be characterized by an X-ray powder diffraction pattern having peaks shown in Table 5 set forth in the Examples. In some embodiments, the succinate crystalline salt form of (S)-mepazine has an X-ray powder diffraction pattern substantially as shown in FIG. 1, wherein by “substantially” is meant that the reported peaks can vary by about ±0.2°. It is well known in the field of XRPD that while relative peak heights in spectra are dependent on a number of factors, such as sample preparation and instrument geometry, peak positions are relatively insensitive to experimental details.

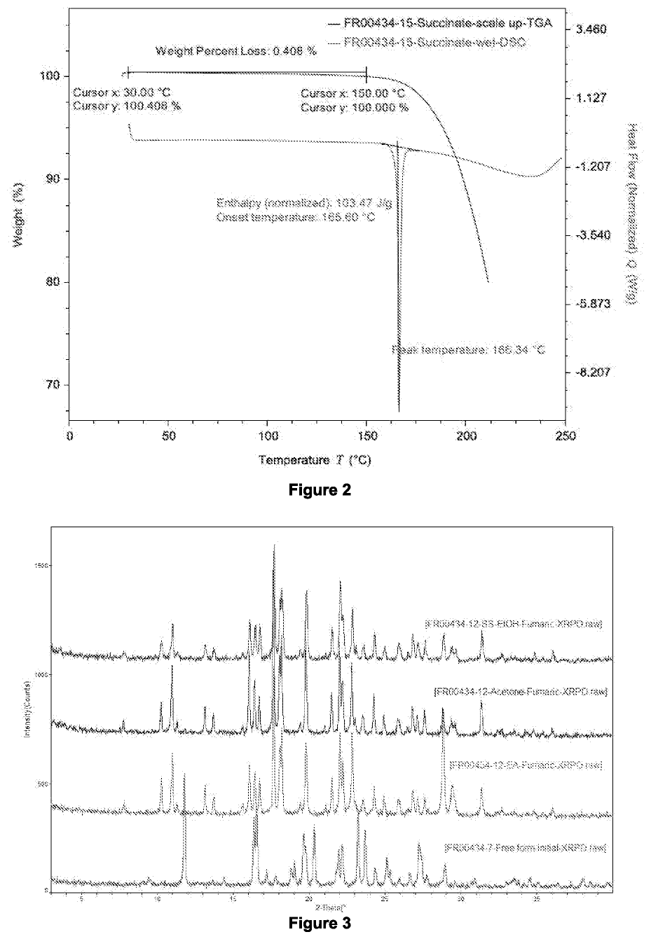

Differential scanning calorimetry (DSC) thermographs were obtained, as set forth in the Examples, for the succinate crystalline salt form of (S)-mepazine. The DSC curve indicates an endothermic transition at about 166° C.±3° C. Thus, in some embodiments, the succinate crystalline salt form of (S)-mepazine can be characterized by a DSC thermograph having an onset temperature in a range of about 156° C. to about 176° C. For example, in some embodiments, the succinate crystalline salt form of (S)-mepazine is characterized by DSC, as shown in FIG. 2.

The succinate crystalline salt form of (S)-mepazine also can be characterized by thermogravimetric analysis (TGA). Thus, the succinate crystalline salt form of (S)-mepazine can be characterized by a weight loss in a range of about 0% to about 1% with an onset temperature in a range of about 145° ° C. to about 155° C. For example, the succinate crystalline salt form of (S)-mepazine can be characterized by a weight loss of about 0.4% between about 60° ° C. to 150° C. In some embodiments, the succinate crystalline salt form of (S)-mepazine has a thermogravimetric analysis substantially as depicted in FIG. 2, wherein by “substantially” is meant that the reported TGA features can vary by about ±5° C.

### Fumarate Salt Crystalline Form

Fumarate crystalline salt form of (S)-mepazine can be characterized by an X-ray powder diffraction pattern, obtained as set forth in the Examples, having peaks at about 17.7, 18.1, and 22.1±0.2° 2θ using Cu Kα radiation. The fumarate crystalline salt form optionally can be further characterized by an X-ray powder diffraction pattern having additional peaks at about 11.0, 16.1, 18.2, 19.8, and 22.9±0.2° 2θ using Cu Kα radiation. The fumarate crystalline salt form optionally can be further characterized by an X-ray powder diffraction pattern having additional peaks at about 10.2, 16.5, 16.8, 21.5, 22.2, and 24.3±0.2° 2θ using Cu Kα radiation. The fumarate crystalline salt form optionally can be further characterized by an X-ray powder diffraction pattern having additional peaks at about 5.5, 7.8, 11.3, 13.2, 13.8, 15.7, 19.4, 21.1, 23.1, 23.6, 25.0, 25.9, 26.9, 27.2, 27.7, 28.9, 29.5, 29.7, 31.4, 32.5, 32.7, 33.6, 34.8, and 36.1±0.2° 2θ using Cu Kα radiation. The fumarate crystalline salt form optionally can be characterized by an X-ray powder diffraction pattern having peaks shown in Table 6 set forth in the Examples. In some embodiments, the fumarate crystalline salt form has an X-ray powder diffraction pattern substantially as shown in FIG. 3, wherein by “substantially” is meant that the reported peaks can vary by about ±0.2°. It is well known in the field of XRPD that while relative peak heights in spectra are dependent on a number of factors, such as sample preparation and instrument geometry, peak positions are relatively insensitive to experimental details.

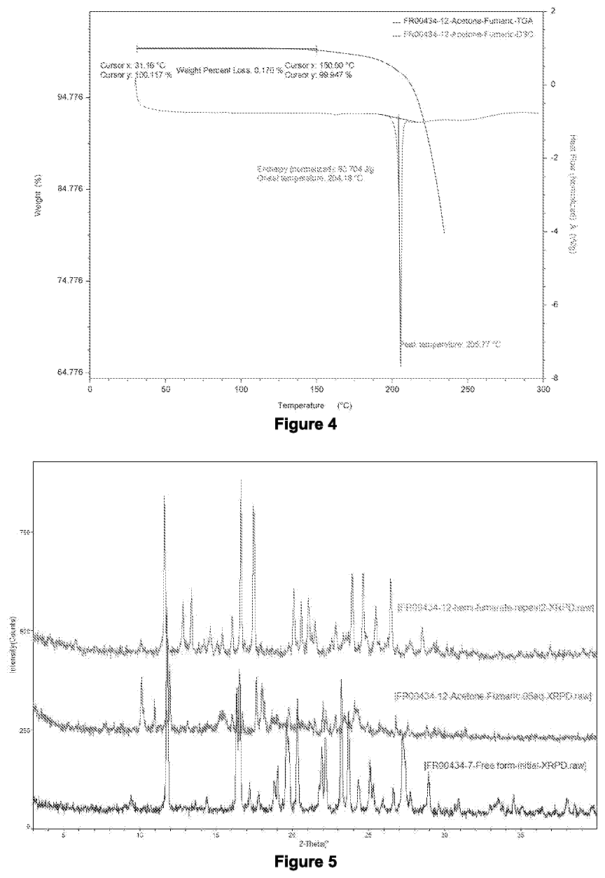

Differential scanning calorimetry (DSC) thermographs were obtained, as set forth in the Examples, for the fumarate crystalline salt form. The DSC curve indicates an endothermic transition at about 204.2° C.±3° C. Thus, in some embodiments, fumarate crystalline salt form can be characterized by a DSC thermograph having a melting endotherm with an onset in a range of about 200° ° C. to about 210° C. For example, in some embodiments, the fumarate crystalline salt form is characterized by DSC, as shown in FIG. 4.

The fumarate crystalline salt form of (S)-mepazine also can be characterized by thermogravimetric analysis (TGA). Thus, the fumarate crystalline salt form of (S)-mepazine can be characterized by a weight loss in a range of about 0% to about 0.5% with an onset temperature in a range of about 145° C. to about 155° C. For example, fumarate crystalline salt form of (S)-mepazine can be characterized by a weight loss of about 0.17%. In some embodiments, the fumarate crystalline salt form of (S)-mepazine has a thermogravimetric analysis substantially as depicted in FIG. 4, wherein by “substantially” is meant that the reported TGA features can vary by about ±5° C.

### Hemi-Fumarate Crystalline Salt Forms I and II

The hemi-fumarate crystalline salt form I of (S)-mepazine can be characterized by an X-ray powder diffraction pattern, obtained as set forth in the Examples, having peaks at about 10.1, 12.0, and 17.7±0.2° 2θ using Cu Kα radiation. The hemi-fumarate crystalline salt form I optionally can be further characterized by an X-ray powder diffraction pattern having additional peaks at about 11.0, 15.5, 18.1, 18.2, 19.8, and 22.0±0.2° 2θ using Cu Kα radiation. The hemi-fumarate crystalline salt form I optionally can be further characterized by an X-ray powder diffraction pattern having additional peaks at about 7.8, 11.8, 13.2, 16.1, 16.5, 16.8, 18.7, 22.2, 22.9, 23.5, 24.2, 24.2, 25.0, 25.8, 26.8, 27.6, 28.9, 30.0, and 31.4±0.2° 2θ using Cu Kα radiation. The hemi-fumarate crystalline salt form I optionally can be characterized by an X-ray powder diffraction pattern having peaks shown in Table 8A set forth in the Examples. In some embodiments, the hemi-fumarate crystalline salt form I has an X-ray powder diffraction pattern substantially as shown in FIG. 5, wherein by “substantially” is meant that the reported peaks can vary by about ±0.2°.

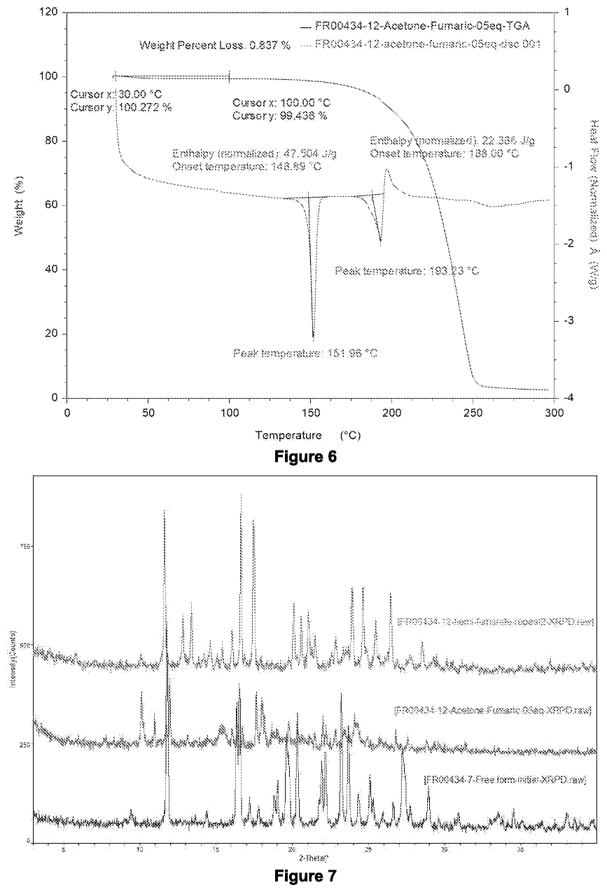

Differential scanning calorimetry (DSC) thermographs were obtained, as set forth in the Examples, for the hemi-fumarate crystalline salt form I. The DSC curve indicates an endothermic transition at about 148.9° C. and 188.0±3° C. Thus, in some embodiments, the hemi-fumarate crystalline salt form I can be characterized by a DSC thermograph having a melting endotherm with an onset in a range of about 145° C. to about 155° C. and about 185° ° C. to about 195° ° C. For example, in some embodiments the hemi-fumarate crystalline salt form I is characterized by DSC, as shown in FIG. 6.

The hemi-fumarate crystalline salt form I also can be characterized by thermogravimetric analysis (TGA). Thus, the hemi-fumarate crystalline salt form I can be characterized by a weight loss in a range of about 0.5% to about 1.5% with an onset temperature in a range of about 95° C. to about 105° C. For example, the hemi-fumarate crystalline salt form I can be characterized by a weight loss of about 0.84%, up to about 100° C. In some embodiments, the hemi-fumarate crystalline salt form I has a thermogravimetric analysis substantially as depicted in FIG. 6, wherein by “substantially” is meant that the reported TGA features can vary by about ±5° C.

The hemi-fumarate crystalline salt form II of (S)-mepazine can be characterized by an X-ray powder diffraction pattern, obtained as set forth in the Examples, having peaks at about 11.7, 16.7, and 17.5±0.2° 2θ using Cu Kα radiation. The hemi-fumarate crystalline salt form II optionally can be further characterized by an X-ray powder diffraction pattern having additional peaks at about 13.4, 20.1, 24.0, 24.7, and 26.5±0.2° 2θ using Cu Kα radiation. The hemi-fumarate crystalline salt form II optionally can be further characterized by an X-ray powder diffraction pattern having additional peaks at about 5.9, 10.1, 12.9, 13.9, 14.2, 14.7, 15.1, 15.5, 16.1, 16.9, 18.0, 21.5, 22.6, 22.9, 24.9, 25.5, 27.8, 28.6, 29.3, 30.1, 31.2, 37.0, and 39.1±0.2° 2θ using Cu Kα radiation. The hemi-fumarate crystalline salt form II optionally can be characterized by an X-ray powder diffraction pattern having peaks shown in Table 8B set forth in the Examples. In some embodiments, the hemi-fumarate crystalline salt form II has an X-ray powder diffraction pattern substantially as shown in FIG. 7, wherein by “substantially” is meant that the reported peaks can vary by about ±0.2°.

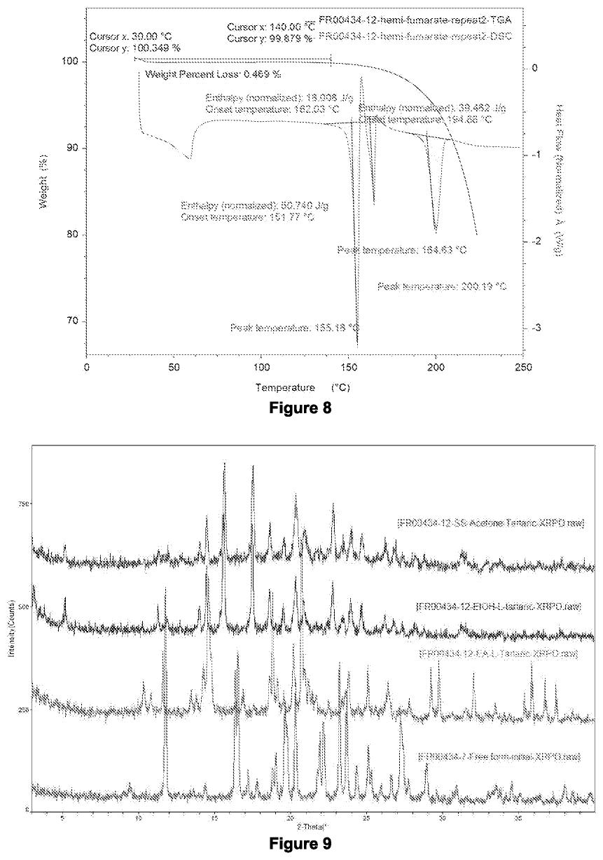

Differential scanning calorimetry (DSC) thermographs were obtained, as set forth in the Examples, for the hemi-fumarate crystalline salt form II. The DSC curve indicates an endothermic transition at about 151.8° C., 162.0° C., and 194.9±3° C. Thus, in some embodiments, the hemi-fumarate crystalline salt form II can be characterized by a DSC thermograph having a melting endotherm with an onset in a range of about 145° C. to about 155° C., about 158° C. to about 165° C., and about 190° ° C. to about 200° ° C. For example, in some embodiments the hemi-fumarate crystalline salt form II is characterized by DSC, as shown in FIG. 8.

The hemi-fumarate crystalline salt form II also can be characterized by thermogravimetric analysis (TGA). Thus, the hemi-fumarate crystalline salt form II can be characterized by a weight loss in a range of about 0.1% to about 1% with an onset temperature in a range of about 120° ° C. to about 140° C. For example, the hemi-fumarate crystalline salt form II can be characterized by a weight loss of about 0.47%, up to about 130° C. In some embodiments, the hemi-fumarate crystalline salt form II has a thermogravimetric analysis substantially as depicted in FIG. 8, wherein by “substantially” is meant that the reported TGA features can vary by about ±5° C.

### Tartrate Crystalline Salt Forms

Tartrate crystalline salt form I of (S)-mepazine can be characterized by an X-ray powder diffraction pattern, obtained as set forth in the Examples, having peaks at about 14.5, 15.6, and 17.5±0.2° 2θ using Cu Kα radiation. The tartrate crystalline salt form I optionally can be further characterized by an X-ray powder diffraction pattern having additional peaks at about 18.6, 20.4, 22.8, 24.0, and 24.7±0.2° 2θ using Cu Kα radiation. The tartrate crystalline salt form I optionally can be further characterized by an X-ray powder diffraction pattern having additional peaks at about 3.1, 3.9, 5.2, 11.3, 14.0, 19.6, 20.9, 22.5, 26.2, and 31.2±0.2° 2θ using Cu Kα radiation. The tartrate crystalline salt form I optionally can be further characterized by an X-ray powder diffraction pattern having additional peaks at about 23.5, 26.8, 28.1, 28.8, and 35.5±0.2° 2θ using Cu Kα radiation. The tartrate crystalline salt form I optionally can be characterized by an X-ray powder diffraction pattern having peaks shown in Table 9 set forth in the Examples. In some embodiments, the tartrate crystalline salt form I has an X-ray powder diffraction pattern substantially as shown in FIG. 9, wherein by “substantially” is meant that the reported peaks can vary by about ±0.2°. It is well known in the field of XRPD that while relative peak heights in spectra are dependent on a number of factors, such as sample preparation and instrument geometry, peak positions are relatively insensitive to experimental details.

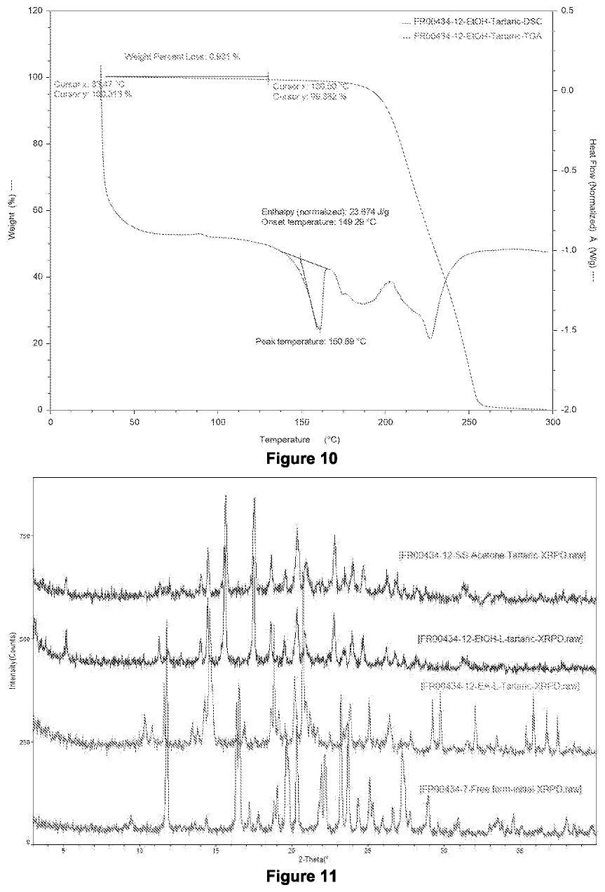

Differential scanning calorimetry (DSC) thermographs were obtained, as set forth in the Examples, for the tartrate crystalline salt form I. The DSC curve indicates an endothermic transition at about 204.2° C.±3° C. Thus, in some embodiments, the tartrate crystalline salt form I can be characterized by a DSC thermograph having a melting endotherm with an onset in a range of about 200° ° C. to about 210° C. For example, in some embodiments, the tartrate crystalline salt form I is characterized by DSC, as shown in FIG. 10.

The tartrate crystalline salt form I of (S)-mepazine also can be characterized by thermogravimetric analysis (TGA). Thus, the tartrate crystalline salt form I of (S)-mepazine can be characterized by a weight loss in a range of about 0% to about 0.5% with an onset temperature in a range of about 145° C. to about 155° C. For example, the tartrate crystalline salt form I of (S)-mepazine can be characterized by a weight loss of about 0.17%. In some embodiments, the tartrate crystalline salt form I of (S)-mepazine has a thermogravimetric analysis substantially as depicted in FIG. 10, wherein by “substantially” is meant that the reported TGA features can vary by about ±5° C.

Tartrate crystalline salt form II of (S)-mepazine can be characterized by an X-ray powder diffraction pattern, obtained as set forth in the Examples, having peaks at about 14.7, 18.9, and 20.8±0.2° 2θ using Cu Kα radiation. The tartrate crystalline salt form II optionally can be further characterized by an X-ray powder diffraction pattern having additional peaks at about 11.6, 20.2, 29.8, 29.3, and 35.9±0.2° 2θ using Cu Kα radiation. The tartrate crystalline salt form II optionally can be further characterized by an X-ray powder diffraction pattern having additional peaks at about 10.4, 13.5, 14.3, 16.9, 18.7, 19.2, 19.6, 20.9, 21.2, 21.7, 23.7, 23.8, 25.1, 26.4, 27.8, 32.0, 33.5, 35.4, 36.7, and 37.5±0.2° 2θ using Cu Kα radiation. The tartrate crystalline salt form II optionally can be further characterized by an X-ray powder diffraction pattern having additional peaks at about 10.9, 13.9, 17.6, 21.5, 22.5, 23.4, 31.5, 34.1, and 38.6±0.2° 2θ using Cu Kα radiation The tartrate crystalline salt form II optionally can be characterized by an X-ray powder diffraction pattern having peaks shown in Table 10 set forth in the Examples. In some embodiments, the tartrate crystalline salt form II has an X-ray powder diffraction pattern substantially as shown in FIG. 11, wherein by “substantially” is meant that the reported peaks can vary by about ±0.2°. It is well known in the field of XRPD that while relative peak heights in spectra are dependent on a number of factors, such as sample preparation and instrument geometry, peak positions are relatively insensitive to experimental details.

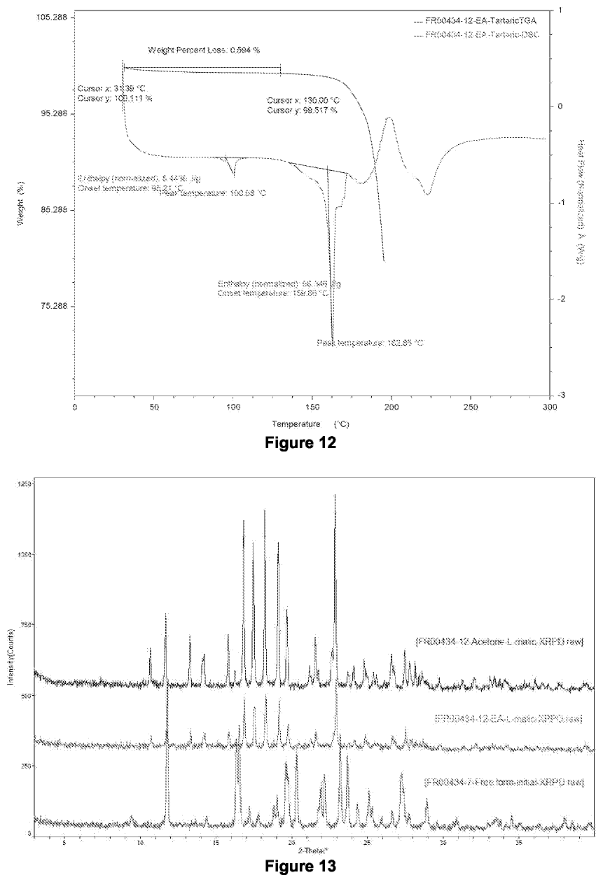

Differential scanning calorimetry (DSC) thermographs were obtained, as set forth in the Examples, for the tartrate crystalline salt form II. The DSC curve indicates an endothermic transition at about 148.9° C. and 188.0° C.±3° C. Thus, in some embodiments, the tartrate crystalline salt form II can be characterized by a DSC thermograph having a melting endotherm with an onset in a range of about 145° C. to about 155° C. and about 185° ° C. to about 195° ° C. For example, in some embodiments, the tartrate crystalline salt form II is characterized by DSC, as shown in FIG. 12.

The tartrate crystalline salt form II of (S)-mepazine also can be characterized by thermogravimetric analysis (TGA). Thus, the tartrate crystalline salt form II of (S)-mepazine can be characterized by a weight loss in a range of about 0.25% to about 0.75% with an onset temperature in a range of about 125° C. to about 135° C. For example, the tartrate crystalline salt form II of (S)-mepazine can be characterized by a weight loss of about 0.59%. In some embodiments, the tartrate crystalline salt form II of (S)-mepazine has a thermogravimetric analysis substantially as depicted in FIG. 12, wherein by “substantially” is meant that the reported TGA features can vary by about ±5° C.

### Malate Crystalline Salt Form

Malate crystalline salt form of (S)-mepazine can be characterized by an X-ray powder diffraction pattern, obtained as set forth in the Examples, having peaks at about 16.9, 18.3, and 23.0±0.2° 2θ using Cu Kα radiation. The malate crystalline salt form optionally can be further characterized by an X-ray powder diffraction pattern having additional peaks at about 13.8, 17.6, 19.2, 19.8, and 27.6±0.2° 2θ using Cu Kα radiation. The tartrate crystalline salt form optionally can be further characterized by an X-ray powder diffraction pattern having additional peaks at about 10.8, 11.8, 14.3, 15.9, 21.3, 21.7, 24.9, 26.7, 27.9, 28.2, and 28.7±0.2° 2θ using Cu Kα radiation. The tartrate crystalline salt form optionally can be further characterized by an X-ray powder diffraction pattern having additional peaks at about 24.2, 25.5, 25.7, 29.8, 31.4, 32.2, 35.5, 36.7, 39.5, and 39.6±0.2° 2θ using Cu Kα radiation. The malate crystalline salt form optionally can be characterized by an X-ray powder diffraction pattern having peaks shown in Table 11 set forth in the Examples. In some embodiments, the malate crystalline salt form has an X-ray powder diffraction pattern substantially as shown in FIG. 13, wherein by “substantially” is meant that the reported peaks can vary by about ±0.2°. It is well known in the field of XRPD that while relative peak heights in spectra are dependent on a number of factors, such as sample preparation and instrument geometry, peak positions are relatively insensitive to experimental details.

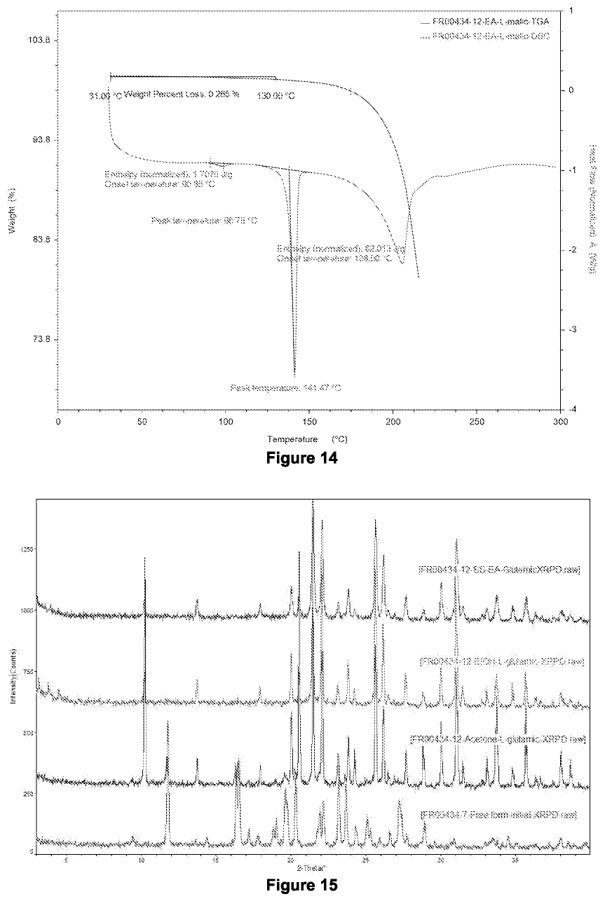

Differential scanning calorimetry (DSC) thermographs were obtained, as set forth in the Examples, for the malate crystalline salt form. The DSC curve indicates an endothermic transition at about 138° C.±3° C. Thus, in some embodiments, the malate crystalline salt form can be characterized by a DSC thermograph having a melting endotherm with an onset in a range of about 135° C. to about 145° C. For example, in some embodiments, the malate crystalline salt form is characterized by DSC, as shown in FIG. 14.

The malate crystalline salt form of (S)-mepazine also can be characterized by thermogravimetric analysis (TGA). Thus, the malate crystalline salt form of (S)-mepazine can be characterized by a weight loss in a range of about 0% to about 0.5% with an onset temperature in a range of about 125° ° C. to about 135° C. For example, the malate crystalline salt form of (S)-mepazine can be characterized by a weight loss of about 0.26%. In some embodiments, the malate crystalline salt form of (S)-mepazine has a thermogravimetric analysis substantially as depicted in FIG. 14, wherein by “substantially” is meant that the reported TGA features can vary by about ±5° C.

### Glutamate Crystalline Salt Form

Glutamate crystalline salt form of (S)-mepazine can be characterized by an X-ray powder diffraction pattern, obtained as set forth in the Examples, having peaks at about 21.5, 22.1, and 25.7±0.2° 2θ using Cu Kα radiation. The glutamate crystalline salt form optionally can be further characterized by an X-ray powder diffraction pattern having additional peaks at about 20.1, 20.6, 23.9, 26.2, and 30.1±0.2° 2θ using Cu Kα radiation. The glutamate crystalline salt form optionally can be further characterized by an X-ray powder diffraction pattern having additional peaks at about 10.3, 13.8, 18.0, 23.2, 27.7, 31.5, 33.8, 34.9, 35.8, and 38.1±0.2° 2θ using Cu Kα radiation. The glutamate crystalline salt form optionally can be further characterized by an X-ray powder diffraction pattern having additional peaks at about 24.3, 26.5, 28.9, 32.8, 33.1, 36.4, 38.7, and 39.4±0.2° 2θ using Cu Kα radiation. The glutamate crystalline salt form optionally can be characterized by an X-ray powder diffraction pattern having peaks shown in Table 12 set forth in the Examples. In some embodiments, the glutamate crystalline salt form has an X-ray powder diffraction pattern substantially as shown in FIG. 15, wherein by “substantially” is meant that the reported peaks can vary by about ±0.2°. It is well known in the field of XRPD that while relative peak heights in spectra are dependent on a number of factors, such as sample preparation and instrument geometry, peak positions are relatively insensitive to experimental details.

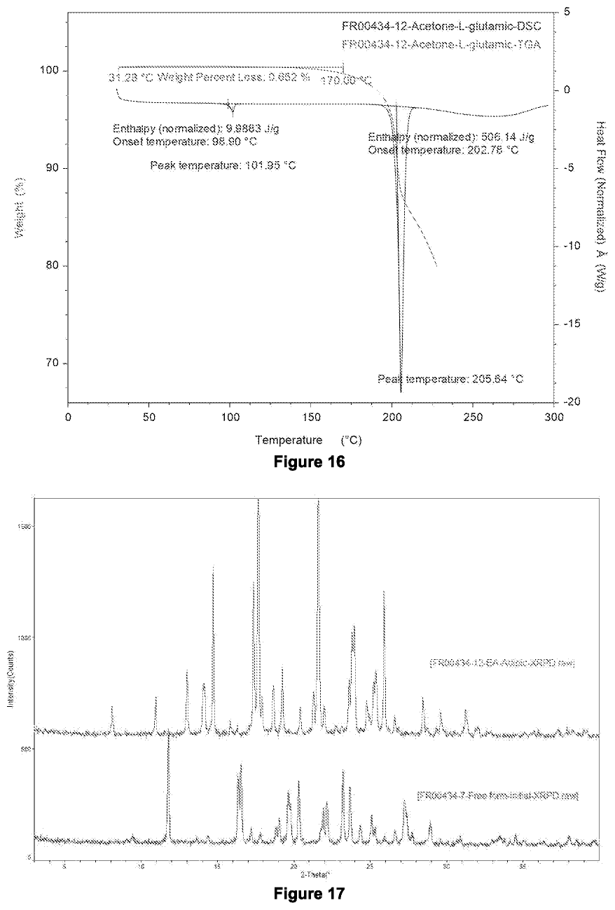

Differential scanning calorimetry (DSC) thermographs were obtained, as set forth in the Examples, for the glutamate crystalline salt form. The DSC curve indicates an endothermic transition at about 98.9° C. and 202.8° C.±3° C. Thus, in some embodiments, the glutamate crystalline salt form can be characterized by a DSC thermograph having a melting endotherm with an onset in a range of about 95° C. to about 105° C. and about 198° C. to about 208° ° C. For example, in some embodiments, the glutamate crystalline salt form is characterized by DSC, as shown in FIG. 16.

The glutamate crystalline salt form of (S)-mepazine also can be characterized by thermogravimetric analysis (TGA). Thus, the glutamate crystalline salt form of (S)-mepazine can be characterized by a weight loss in a range of about 0.35% to about 0.95% with an onset temperature in a range of about 165° C. to about 175° C. For example, the glutamate crystalline salt form of (S)-mepazine can be characterized by a weight loss of about 0.65%. In some embodiments, the glutamate crystalline salt form of (S)-mepazine has a thermogravimetric analysis substantially as depicted in FIG. 16, wherein by “substantially” is meant that the reported TGA features can vary by about ±5° C.

### Adipate Crystalline Salt Form

Adipate crystalline salt form of (S)-mepazine can be characterized by an X-ray powder diffraction pattern, obtained as set forth in the Examples, having peaks at about 14.8, 17.7, and 21.6±0.2° 2θ using Cu Kα radiation. The adipate crystalline salt form optionally can be further characterized by an X-ray powder diffraction pattern having additional peaks at about 13.0, 17.4, 19.3, 23.9, 24.8, and 25.9±0.2° 2θ using Cu Kα radiation. The adipate crystalline salt form optionally can be further characterized by an X-ray powder diffraction pattern having additional peaks at about 14.2, 18.7, 23.7, 25.3, and 25.4±0.2° 2θ using Cu Kα radiation. The adipate crystalline salt form optionally can be further characterized by an X-ray powder diffraction pattern having additional peaks at about 8.1, 11.0, 15.9, 16.3, 17.9, 20.5, 21.3, 22.0, 22.8, 23.2, 24.8, 26.6, 26.9, 28.5, 28.8, 29.4, 29.6, 31.3, 32.0, 32.1, 32.8, 35.8, 36.0, 37.3, 37.9, 39.0, and 39.2±0.2° 2θ using Cu Kα radiation. The adipate crystalline salt form optionally can be characterized by an X-ray powder diffraction pattern having peaks shown in Table 7 set forth in the Examples. In some embodiments, the adipate crystalline salt form has an X-ray powder diffraction pattern substantially as shown in FIG. 17, wherein by “substantially” is meant that the reported peaks can vary by about ±0.2°. It is well known in the field of XRPD that while relative peak heights in spectra are dependent on a number of factors, such as sample preparation and instrument geometry, peak positions are relatively insensitive to experimental details.

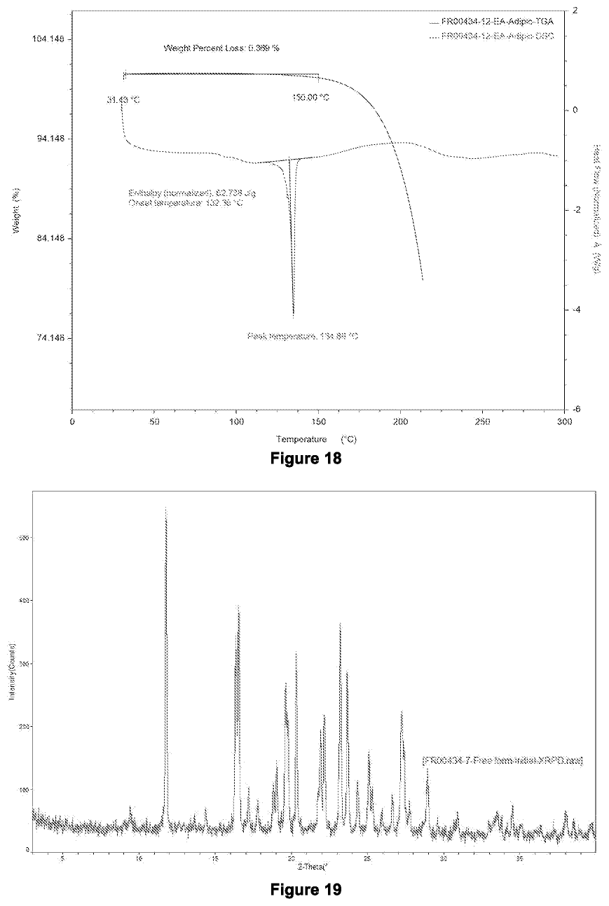

Differential scanning calorimetry (DSC) thermographs were obtained, as set forth in the Examples, for the adipate crystalline salt form. The DSC curve indicates an endothermic transition at about 132.4° C. Thus, in some embodiments, the adipate crystalline salt form can be characterized by a DSC thermograph having a melting endotherm with an onset in a range of about 128° ° C. to about 138° C. For example, in some embodiments, the adipate crystalline salt form is characterized by DSC, as shown in FIG. 18.

The adipate crystalline salt form of (S)-mepazine also can be characterized by thermogravimetric analysis (TGA). Thus, the adipate crystalline salt form of (S)-mepazine can be characterized by a weight loss in a range of about 0.15% to about 0.55% with an onset temperature in a range of about 125° C. to about 135° C. For example, the adipate crystalline salt form of (S)-mepazine can be characterized by a weight loss of about 0.37%. In some embodiments, the adipate crystalline salt form of (S)-mepazine has a thermogravimetric analysis substantially as depicted in FIG. 18, wherein by “substantially” is meant that the reported TGA features can vary by about ±5° C.

International Patent Application No. WO 2014/207067 discloses a hydrochloride salt of (S)-mepazine. That hydrochloride salt form of (S)-mepazine, however, is extremely hygroscopic under humid conditions. For example, under humid conditions, 25° C. and 95% RH for about 5 hours by DVS, the hydrochloride salt form of (S)-mepazine absorbs 16.9 wt % moisture, and under long term humidity conditions, 25° C. and 92.5% RH for one week by DVS, the hydrochloride salt form of (S)-mepazine is deliquescent. Such hygroscopicity is not desirable for a drug product, as it can lead to instability or degradation upon storage (e.g., dissociate to free base drug form), and/or lack of precision on measured amount of drug in a drug product, e.g. significant error in a drug assay by HPLC, particularly if the reference standard has a significantly different amount of water. A drug substance having high hygroscopicity, such as the hydrochloride salt of (S)-mepazine, can induce or facilitate unwanted chemical reactions leading to drug substance degradation and/or change in color and appearance. In contrast, the organic diacid salt forms of (S)-mepazine disclosed herein consistently perform well in non-humid and humid conditions of 0% RH to 95% RH at 25° C. In various embodiments, the organic diacid salt forms absorb less than 5 wt % moisture, or less than 3 wt % moisture, or less than 2 wt % moisture, or less than 1 wt % moisture. A non-hygroscopic or low hygroscopic drug substance, such as the organic acid salt form of (S)-mepazine disclosed herein, provides benefits including, but not limited to, consistency of manufacturing and assays of the drug product or formulation, as well as less weight gain over extended storage time such that the facilitating of unwanted chemical reactions or color/appearance changes from the water gain does not occur. In particular, the succinate salt form of (S)-mepazine exhibits good physical properties for dissolution, but does not dissociate to the (S)-mepazine free base. A lower hygroscopicity of the succinate salt form allows for easier isolation and purification of the salt form. Moreover, the lower the hygroscopicity of the succinate salt form, the easier to formulate a pharmaceutical formulation involving water as a binder solution or processing aid.

### Synthesis of (S)-Mepazine

Provided herein are processes for synthesizing (S)-mepazine, or a salt thereof.

International Patent Application No. WO 2014/207067 discloses a synthesis of (S)-mepazine, and a hydrochloride salt thereof. The synthesis provided there, however, has many shortcomings, such as, the isolation of the tosylated n-heterocycle (e.g., (S)-3-tosylmethyl-1-methylpiperidine), referred to as an example of compound (K) herein, proved to be challenging, as the tosylated n-heterocycle is unstable over time. The tosylated n-heterocycle contains both a nucleophilic site (tertiary amine) and an electrophilic site (the adjacent carbon to the tosylate), thus oligomerization/polymerization or elimination reactions are expected to occur overtime resulting in an impure product. Further, the tosylated n-heterocycle was an oil, which is undesirable for intermediates in the production of API's. Advantages of the methods disclosed herein include one or more of (A) safer method due to the use of safer reagents and waste products by avoiding synthesis of less stable intermediates and reagents; (B) facile isolation of the intermediates, for example, step (b) provides a salt form intermediate which can easily be prepared as a solid and/or is crystallized which solves the instability issue described above for compound (K); and (C) improved overall purity of the product, for example, by decreased work-up, which also allows for decreased costs through decreased chemicals/materials used in the synthesis. Provided herein are processes for synthesizing (S)-mepazine, or a salt thereof, comprising
(a) admixing compound (J), a base, and a leaving group reagent in a solvent to form compound (K):

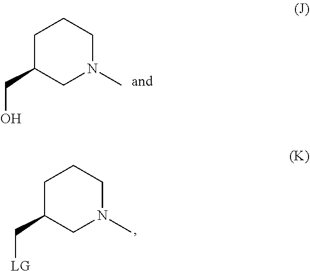

wherein LG is leaving group;
(b) forming a hydrochloride salt of compound (K); and
(c) admixing the hydrochloride salt of compound (K) and phenothiazine in a solvent to form (S)-mepazine.

A general reaction scheme for the processes described herein is provided in Scheme 1, below.

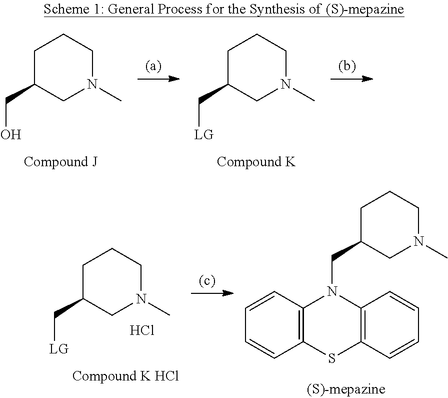

Step (a)—Leaving Group (“LG”) Addition

The processes of the disclosure include reaction of compound J with a leaving group reagent and a base in a solvent to provide compound K. Compound J is reacted with a leaving group reagent and an amine base, which forms compound K. As used here, the leaving group (LG) refers to any suitable atom or functional group that can be displaced by a nucleophile during a nucleophilic substitution reaction. Leaving group reagents that can convert a hydroxyl group to a leaving group to make nucleophilic substitution favorable are well known in the art. Nonlimiting examples of suitable leaving groups include halides, such as CI, Br, or I, or sulfonates as discussed below.

In some embodiments, LG is a sulfonate leaving group. As used herein, the term “sulfonate leaving group” refers to a leaving group in which the oxygen atom of a hydroxyl group is bound to a sulfonyl group—

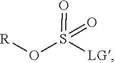

where R—O is derived from the hydroxyl group being converted to a leaving group and LG′ is derived from the rest of the sulfonyl leaving group. In some embodiments, the sulfonate leaving group is selected from the group consisting of mesylate, tosylate, nosylate, and triflate. In some embodiments, the sulfonyl leaving group comprises tosylate.

In general, the leaving group reagent can be any suitable leaving group reagent known to one of ordinary skill in the art as is used to convert a hydroxyl group to a leaving group. In embodiments the leaving group reagent comprises mesyl chloride, tosyl chloride, nosyl chloride, methanesulfonic anhydride, para-toluenesulfonic anhydride, or a combination thereof. In some embodiments, the leaving group reagent can comprise 4-toluenesulfonyl chloride (“tosyl chloride”).

The leaving group reagent and compound J can be present in a molar ratio of 1:0.9 to 1:2, for example, at least a molar ratio of 1:0.9, 1:1, 1:1.1, 1:1.2, 1:1.5, 1:1.6 and/or up to 1:0.9, 1:1.2, 1:1.75, 1:1.5, such as 1:1.2 to 1:1.9, 1:1.2 to 1:7, or 1:1.2 to 1:1.5.

The admixing of step (a) occurs in the presence of a solvent. In some embodiments, the solvent is an organic solvent. In various embodiments, the organic solvent comprises dichloromethane, tetrahydrofuran, 2-methyltetrahydrofuran, dibutyl ether, methyl tert-butyl ether, diisopropyl ether, diethyl ether, chloroform, dimethylformamide, dimethylacetamide, N-methyl-2-pyrrolidone, 1,4-dioxane, or a combination thereof. In some embodiments, the solvent comprises 2-methyltetrahydrofuran.

The admixing of step (a) occurs in the presence of a base. In some embodiments, the base is an amine base (e.g., mono-, di-, or trialkylamine, substituted or unsubstituted piperidine, substituted or unsubstituted pyridine). In some embodiments, the amine base comprises pyridine, 4-dimethylaminopyridine, trimethylamine, triethylamine, aniline, diisopropylethylamine, 1,8-diazabicyclo [5.4.0] undec-7-ene (DBU), 1,4-diazabicyclo [2.2.2] octane (DABCO), 2,6-lutidine, or a combination thereof. In some embodiments, the amine base comprises a trialkyl amine. In some embodiments, the amine base is triethylamine.

Compound J and the amine base can be present in a molar ratio of 1:0.9 to 1:3.3, for example, at least a molar ratio of 1:0.9, 1:1, 1:1.1, 1:1.2, 1:1.5, 1:1.7, 1:1.8, 1:2, 1:2.5 and/or up to 1:3.3, 1:3, 1:2.5, 1:2, 1:1.1, such as 1:1.8 to 1:3, 1:2 to 1:3, or 1:2 to 1:2.5.

In some embodiments, the admixing of step (a) can occur for 30 minutes to 48 hours or longer. In embodiments, the admixing of step (a) can occur for 30 minutes to 36 hours, 30 minutes to 24 hours, 30 minutes to 3 hours, 1.5 hours to 2.5 hours, 6 hours to 20 hours, 12 hours to 24 hours, 12 hours to 20 hours, 12 hour to 18 hours, 15 hours to 20 hours, or 16 hours to 18 hours.

The admixing of step (a) can occur at a temperature of −10° C. to 30° C., for example at least −10, −5, 0, or 5 and/or up to 30, 25, 20, 15, 10, 7, 5, 0, or −10, such as −10° ° C. to 5° ° C., −10° C. to 10° C., −5° C. to 5° C., −5° ° C. to 10° C., 0° ° C. to 10° ° C., 0° C. to 15° C., 0° C. to 20° ° C., −10° ° C. to 25° ° C., 0° C. to 25° C. In some embodiments, the admixing occurs at a temperature of −10° ° C. to 25° C.

### Step (b) Formation of HCl Salt

The processes of the disclosure include formation of a HCl salt from compound K to provide compound K hydrochloride salt. Formation of the HCl salt of compound K can occur using any suitable reaction conditions. In some cases, compound K is reacted with HCl to form compound K hydrochloride salt. In some embodiments, the reaction between compound K and HCl occurs in the presence of a solvent, such as an organic solvent. In some embodiments, the organic solvent comprises dichloromethane, tetrahydrofuran, 2-methyltetrahydrofuran, tert-butyl methyl ether, diethyl ether, chloroform, ethyl acetate, isopropyl acetate, butyl acetate, 1,4-dioxane, or a combination thereof. In some embodiments, the solvent comprises 2-methyltetrahydrofuran.

In some embodiments, the admixing of step (b) can occur for 30 minutes to 48 hours or longer. In embodiments, the admixing of step (b) can occur for 30 minutes to 36 hours, 30 minutes to 24 hours, 30 minutes to 4 hours, 1 hour to 4 hours, 2 hours to 3 hours, 6 hours to 20 hours, 12 hours to 24 hours, 12 hours to 20 hours, 12 hour to 18 hours, 15 hours to 20 hours, or 16 hours to 18 hours.

The admixing of step (b) can occur at a temperature of −10° C. to 30° C., for example at least −10, −5, 0, 10 or 5 and/or up to 30, 25, 20, 15, 10, 7, 5, 0, or −10, such as −10° C. to 5° C., −10° C. to 10° C., −5° C. to 20° C., −5° ° C. to 15° C., 0° C. to 15° ° C., 0° C. to 20° C., 10° C. to 20° C., −10° C. to 25° C., 0° C. to 25° C. In some embodiments, the admixing occurs at a temperature of 10° ° C. to 20° C.

Compound K and hydrochloric acid can be present in a molar ratio of 1:0.9 to 1:5, for example, at least a molar ratio of 1:1.2, 1:1.5, 1:1.6, 1:2, 1:3, and/or up to 1:5, 1:3.5, such as 1:1.2 to 1:2, 1:2.5 to 1:3.5, or 1:1.2 to 1:3.5. In some embodiments, the molar ratio of compound K and the hydrochloric acid is 1:3.

In some embodiments, step (b) further comprises crystallizing the hydrochloride salt of compound (K). In some embodiments, the crystalline hydrochloride salt of compound (K) is also isolated, e.g., by filtration, centrifugation, or both. In some embodiments, step (b) further comprises isolating the hydrochloride salt of compound (K) by filtration.

Compound K hydrochloride salt as formed in step (b) can be used directly in reaction with phenothiazine (i.e., step (c)) without the need for substantial purification. In some embodiments, compound K hydrochloride salt is crystallized, washed, and is then substantially pure for use in the next steps. Advantageously, the generation of the compound K hydrochloride salt provides compound K without the need for excessive purification and workup steps. Moreover, the generation of the compound K hydrochloride salt provides for less impurities, because the free base of compound K is unstable towards elimination of the leaving group, e.g., toluenesulfonic acid, to form the olefin, whereas the compound K hydrochloride salt has better stability towards elimination.

Step (c)—Nucleophilic Substitution

The processes of the disclosure include the nucleophilic substitution of compound K hydrochloride salt with phenothiazine to provide (S)-mepazine. The processes herein comprise admixing the hydrochloride salt of compound K and phenothiazine in a solvent to form (S)-mepazine.

The reaction between compound K hydrochloride salt and phenothiazine occurs in the presence of a solvent. In some embodiments, the solvent is an organic solvent. In some embodiments, the solvent is a polar aprotic solvent. In various embodiments, the polar aprotic solvent comprises dichloromethane, tetrahydrofuran, 2-methyltetrahydrofuran, tert-butyl methyl ether, diethyl ether, chloroform, dimethyl sulfoxide, 1,4-dioxane, dimethyl formamide, pyridine, N-methyl-2-pyrrolidone (“NMP”), dimethylacetamide, or a combination thereof. In some embodiments, the polar aprotic solvent comprises dimethyl formamide, dimethyl acetamide, N-methyl-2-pyrrolidone, or a combination thereof. In some embodiments, the solvent is an amine solvent. Contemplated amine solvents include pyridine, triethylamine, diisopropylethylamine, 2,6-lutidine, and N-methylmorpholine. In some embodiments, the solvent comprises NMP.

The admixing of step (c) can occur for 30 minutes to 48 hours or longer. In some embodiments, the admixing of step (c) occurs for 1 hour to 36 hours, 1 hour to 24 hours, 6 hours to 20 hours, 12 hours to 24 hours, 12 hours to 20 hours, 12 hours to 18 hours, 15 hours to 20 hours, or 16 hours to 20 hours.

In some embodiments, the admixing of step (c) occurs at room temperature. In embodiments, the admixing of step (c) occurs at a temperature of 10-30° C., such as 20-30° ° C. or 15-25° C.

Compound K hydrochloride salt and phenothiazine can be present in a molar ratio of 1:0.9 to 1:2, for example, at least a molar ratio of 1:0.9, 1:1, 1:1.2, 1:1.5, 1:1.6 and/or up to 1:2, 1:1.75, 1:1.5, such as 1:1.2 to 1:1.9, 1:1.2 to 1:7, or 1:1.2 to 1:1.5.

In some embodiments, step (c) further comprises a base. In some embodiments, the base comprises one or more of a hydride, an organolithium reagent, and a Grignard reagent. In some embodiments, the organolithium reagent comprises one or more of methyl lithium, ethyl lithium, tert-butyl lithium, lithium hexamethyldisilylazide, and lithium diisopropylamide. In embodiments, the organolithium reagent can include alkali counterions other than lithium, such as sodium, potassium, rubidium, or cesium. A “Grignard reagent” has a structure of R-MgQ, wherein Q is a halogen (e.g., CI, Br, or l) and R is an alkyl or aryl group (e.g., methyl or phenyl). In embodiments, the Grignard reagent can be complexed with a lithium halide, such as, isopropylmagnesium chloride/lithium chloride. In some embodiments, the base comprises lithium hydride, sodium hydride, potassium hydride, or a combination thereof. In embodiments, the base comprises sodium hydride.

Compound K hydrochloride salt and the base can be present in a molar ratio of 1:0.9 to 1:5, for example, at least a molar ratio of 1:1.5, 1:2, 1:2.5 and/or up to 1:5, 1:4, 1:3, 1:2.5, or 1:1.5 such as 1:0.9, 1:1, 1:1.1, 1:1.2, 1:5, 1:4, 1:3, or 1:2.5.

Step (d)—Organic Diacid Salt Formation

In some embodiments, the processes of the disclosure further comprise the formation of an organic diacid salt of (S)-mepazine, step (d). In some embodiments, the processes disclosed herein comprise admixing (S)-mepazine with an organic diacid to form a salt of structure

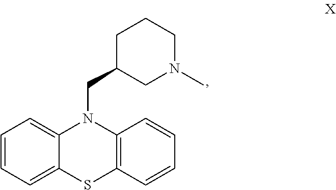 [SMILES: CN1CCC[C@H](CN2c3ccccc3Sc3ccccc32)C1]

wherein X comprises the conjugate base of the organic diacid. In some embodiments, the organic diacid is succinic acid, fumaric acid, tartaric acid, malic acid, glutamic acid, or adipic acid. In some embodiments, the organic diacid is succinic acid, and X is succinate. In some embodiments, the organic diacid is fumaric acid, and X is fumarate or hemi-fumarate. In some embodiments, the organic diacid is tartaric acid, and X is tartrate.

(S)-mepazine and the organic diacid can be present in a molar ratio of 1:0.5 to 1:2.5, for example, at least a molar ratio of 1:0.5, 1:1, 1:1.1, 1:1.2, 1:1.5, 1:1.6 and/or up to 1:2.5, 1:1.75, 1:1.5, such as 1:0.5, 1:0.8, 1:1, 1:1.1, 1:1.2 to 1:1.2.5, 1:1.2 to 1:7, or 1:1.2 to 1:1.5. In some embodiments, the molar ratio of (S)-mepazine and the organic diacid is 1:1. In some embodiments, the molar ratio of (S)-mepazine and the organic diacid is 1:0.5.

The admixing of step (d) can occur for 30 minutes to 8 hours or longer. In some embodiments, the admixing of step (d) can occur for 30 minutes to 5 hours, 1 hour to 3 hours, 1.5 hours to 2.5 hours, or about 2 hours.

The admixing of step (d) can occur at a temperature of 20° C. to 80° ° C., for example at least 20, 25, 30, or 45 and/or up to 80, 65, 50, or 40 such as 30° C. to 75° ° C., 40° C. to 60° ° C., 45° C. to 55° C., or 50° C. In some embodiments, the admixing occurs at a temperature of 45° ° C. to 55° C.

The reaction between (S)-mepazine and the organic diacid can occur in a solvent. In some embodiments, the solvent comprises an organic solvent, water, or both. In some embodiments, the organic solvent comprises methanol, ethanol, acetone, ethyl acetate, isopropyl acetate, butyl acetate, dichloromethane, tetrahydrofuran, 2-methyltetrahydrofuran, tert-butyl methyl ether, diethyl ether, chloroform, 1,4-dioxane, or a combination thereof. In some embodiments, the solvent comprises acetone. In some embodiments, the solvent comprises ethanol. In some embodiments, the solvent comprises ethanol and water.

Step (d) can further comprise crystallizing the organic diacid salt of (S)-mepazine. In some embodiments, crystallization of the organic diacid salt of (S)-mepazine comprises heating a solution of the organic diacid salt of (S)-mepazine, then cooling the solution (e.g., to room temperature or lower) and allowing the solution to crystallize over 15 minutes to 3 days or longer. In some embodiments, the crystallization step can further comprise adding seed crystals of the organic diacid salt of (S)-mepazine. In embodiments, heating the solution can occur at a temperature of 20° ° C. to 80° C., for example at least 20, 25, 30, or 45 and/or up to 80, 65, 50, or 40 such as 30° ° C. to 75° C., 40° C. to 60° ° C., 45° ° C. to 55° C., or 50° ° C. In some embodiments, heating the solution occurs at a temperature of 45° C. to 55° C., such as 50° C.

In some embodiments, step (d) further comprises isolating the crystalline organic diacid salt of (S)-mepazine.

### Pharmaceutical Formulations

Also provided herein are pharmaceutical formulations comprising (S)-mepazine or a pharmaceutically acceptable salt thereof, and an excipient, in the form of a tablet. A pharmaceutically acceptable salt of (S)-mepazine can be one as discussed herein. The pharmaceutical formulations disclosed herein can comprise (S)-mepazine or a pharmaceutically acceptable salt thereof present in an amount of 10% w/w to 50% w/w in the formulation. In embodiments, the pharmaceutical formulation comprise (S)-mepazine or a pharmaceutically acceptable salt thereof present in an amount of 15% w/w to 45% w/w, or 20% w/w to 40% w/w, or 15% w/w to 35% w/w, or 15% w/w to 30% w/w, or 20% w/w to 30% w/w, 22.5% w/w to 27.5% w/w, or 30% w/w to 40% w/w, in the formulation. In embodiments, the pharmaceutical formulation comprise (S)-mepazine or a pharmaceutically acceptable salt thereof present in an amount of 22.5% w/w to 27.5% w/w in the formulation.

“Pharmaceutically acceptable excipient” or as used herein, “excipients” refers to a broad range of ingredients that may be combined with a compound or salt of the present invention to prepare a pharmaceutical composition or formulation. Excipients are additives that are included in a formulation because they either impart or enhance the stability, delivery and manufacturability of a drug product, and are physiologically innocuous to the recipient thereof. Regardless of the reason for their inclusion, excipients are an integral component of a drug product and therefore need to be safe and well tolerated by patients. Given the teachings and guidance provided herein, those skilled in the art will readily be able to vary the amount or range of excipient without increasing viscosity to an undesirable level. Excipients may be chosen to achieve a desired bioavailability, desired stability, resistance to aggregation or degradation or precipitation, protection under conditions of freezing, lyophilization or high temperatures, or other properties. Typically, excipients include, but are not limited to, diluents, colorants, vehicles, anti-adherants, glidants, disintegrants, flavoring agents, coatings, binders, sweeteners, lubricants, sorbents, preservatives, wetting agents, surfactants, anti-tacking agents, flow aids, and the like. Examples of suitable excipients are well known to the person skilled in the art of tablet formulation and may be found e.g. in Handbook of Pharmaceutical Excipients (eds. Rowe, Sheskey & Quinn), 6th edition 2009.

The excipients as disclosed herein can comprise one or more of a filler, lubricant, binder, disintegrant, flow aid, wetting agent, and anti-tacking agent. In various embodiments, the excipient comprises a lubricant. Examples of contemplated lubricants and flow aids include, but are not limited to, magnesium stearate, calcium stearate, stearic acid, hydrogenated vegetable oil, glyceryl palmitostearate, glyceryl behenate, sodium stearyl fumarate, colloidal silicon dioxide, and talc. The amount of lubricant in a tablet can generally be between 0.25-3% by weight. In various embodiments, the excipient comprises a filler. Examples of contemplated fillers include, but are not limited to, starches, maltodextrins, polyols (such as lactose), and celluloses. Tablets provided herein may include lactose and/or microcrystalline cellulose. Lactose can be used in anhydrous or hydrated form (e.g. monohydrate), and is typically prepared by spray drying, fluid bed granulation, or roller drying. Examples of disintegrants include, but are not limited to, starches, celluloses (e.g., microcrystalline cellulose), cross-linked PVP, sodium starch glycolate, croscarmellose sodium, etc. Examples of binders include, but are not limited to, cross-linked PVP, HPMC, microcrystalline cellulose, sucrose, starches, etc. Examples of anti-tacking agents include, but are not limited to, silica, sodium bicarbonate, or the like. Examples of wetting agents include, but are not limited to, ionic surfactants (including both cation and anionic) and non-ionic surfactants, such as sodium lauryl sulfate or cocamidropropyl betaine. In embodiments, the surfactant is an non-ionic surfactant. In embodiments, the surfactant is not an anionic surfactant with a sulfonate or sulfate, such as sodium lauryl sulfate, ammonium lauryl sulfate, ammonium laureth sulfate, sodium myristyl sulfate, or sodium myreth sulfate.

In various embodiments, the excipient comprises lactose, cellulose, microcrystalline cellulose, dibasic calcium phosphate, mannitol, croscarmellose sodium, sodium starch glycolate, hydroxyl propyl cellulose, magnesium stearate, colloidal silicon dioxide, sodium stearyl fumarate, hydroxyl propyl methyl cellulose (HPMC), polyethylene oxide, talc or a combination thereof. In some embodiments, the excipient comprises lactose, cellulose, microcrystalline cellulose, croscarmellose sodium, colloidal silicon dioxide, hydroxyl propyl cellulose, magnesium stearate, or a combination thereof. In some embodiments, the excipient comprises each of lactose, microcrystalline cellulose, croscarmelose sodium, colloidal silicon dioxide, hydroxyl propyl cellulose, talc, and magnesium stearate.

In some embodiments, the pharmaceutical formulation does not include a sodium lauryl sulfate(SLS). Compared to a pharmaceutical formulation including an API with a similar structure (Thioridazine) that performs well with sodium lauryl sulfate in its formulation, the pharmaceutical formulations disclosed herein unexpectedly do not have increased dissolution but instead the dissolution decreases dramatically compared to pharmaceutical formulations disclosed herein without SLS (FIG. 27).

In various embodiments, the excipient is present in the pharmaceutical formulation in an amount of about 50% w/w to about 90% w/w. In some embodiments, the excipient present in an amount of about 50% w/w to about 80% w/w, or about 50% w/w to about 75% w/w, or about 55% w/w to about 70% w/w, or about 70% w/w to about 80% w/w, or about 60% w/w to about 70% w/w. In various embodiments, the excipient is present in an amount of about 70% w/w to about 80% w/w.

In some embodiments, the pharmaceutical formulation is in the form of an immediate release tablet. An immediate release tablet is one that releases or dissolves at least 80% of the active pharmaceutical ingredient (API) in the tablet within 6 hours. In some embodiments, at least 90% of the API is released or dissolved from the formulation within 6 hours. In some embodiments, at least 50% of the API is released or dissolved from the formulation within 4 hours. In embodiments, at least 90% of the API is released or dissolved within 2 hours. In embodiments, at least 85% of API is released or dissolved within 45 minutes. Assessment of release profile can be assessed using an assay as described in the FDA Guidelines (“Dissolution Testing of Immediate Release Solid Oral Dosage Forms”, issued August 1997, Section IV-A) or as provided in the Examples below.

The pharmaceutical formulations disclosed herein provide benefits, including, but not limited to, better bioavailability than currently known pharmaceutical formulations or extended release formulations, a lower Cmax (e.g., about 90 to 150 (ng/ml), a delayed Tmax (e.g., about 1.5 hours to 3 hours), improved stability of the API, and reproducible dissolution (reduced dissolution variability).

In various embodiments, at least 90% of the (S)-mepazine or salt thereof in the tablet is released or dissolved within 2 hours. In some embodiments, at least 90% of the (S)-mepazine or salt thereof is released or dissolved within 1 hour. In some embodiments, at least 99% of the (S)-mepazine or salt thereof is released or dissolved within 1 hour. In some embodiments, at least 90% of the (S)-mepazine or salt thereof is released or dissolved within 40 minutes. In some embodiments, at least 99% of the (S)-mepazine or salt thereof is released or dissolved within 40 minutes. In some embodiments, at least 90% of the (S)-mepazine or salt thereof is released or dissolved within 30 minutes. In some embodiments, at least 99% of the (S)-mepazine or salt thereof is released or dissolved within 30 minutes.

In various embodiments, upon storage at 40° C.±2° C. and 75% relative humidity (RH)±5% RH in an open container for 4 weeks, the pharmaceutical formulation comprises at least 99% of the (S)-mepazine or salt thereof that was initially present in the formulation. In other words, the (S)-mepazine or salt thereof does not degrade or form a byproduct upon storage under the conditions noted. In some embodiments, at least 99.5% of the (S)-mepazine or salt thereof remains, upon storage at 40° C.±2° C. and 75% relative humidity (RH)±5% RH in an open container for 4 weeks. In some embodiments, at least 99.9% of the (S)-mepazine or salt thereof remains, upon storage at 40° C.±2° C. and 75% relative humidity (RH)±5% RH in an open container for 4 weeks.

In some embodiments, the pharmaceutical formulations disclosed herein further comprise up to 0.5 wt % of (S)-mepazine sulfoxide. In some embodiments, the pharmaceutical formulations disclosed herein further comprise up to 0.4 wt %, 0.3 wt %, 0.2 wt %, 0.1 wt % or less of (S)-mepazine sulfoxide.

The pharmaceutical formulations can be included in a container, pack, or dispenser together with instructions for administration.

### Methods of Use

The (S)-mepazine salts disclosed herein or the pharmaceutical formulations disclosed herein, may be used in the treatment or prevention of cancer, including but not limited to: carcinoma, a melanoma, a sarcoma, a myeloma, a leukemia, or a lymphoma. (S) In some embodiments, the cancer is a carcinoma, a melanoma, a sarcoma, a myeloma, a leukemia, or a lymphoma. In some embodiments, the cancer is a melanoma, colon cancer ovarian cancer, prostate cancer or cervical cancer.

In some embodiments, the cancer is a solid tumor. In some embodiments, the solid tumor is an Adrenocortical Tumor, an Alveolar Soft Part Sarcoma, a Chondrosarcoma, a Colorectal Carcinoma, a Desmoid Tumors, a Desmoplastic Small Round Cell Tumor, an Endocrine Tumors, an Endodermal Sinus Tumor, an Epithelioid Hemangioendothelioma, a Ewing Sarcoma, a Germ Cell Tumors (Solid Tumor), a Giant Cell Tumor of Bone and Soft Tissue, a Hepatoblastoma, a Hepatocellular Carcinoma, a Melanoma, a Nephroma, a Neuroblastoma, a Non-Rhabdomyosarcoma Soft Tissue Sarcoma (NRSTS), an Osteosarcoma, a Paraspinal Sarcoma, a Renal Cell Carcinoma, a Retinoblastoma, a Rhabdomyosarcoma, a Synovial Sarcoma, or a Wilms Tumor.

The (S)-mepazine salts disclosed herein or the pharmaceutical formulations disclosed herein, may be used in the treatment or prevention of an immune disease, such as allergic inflammation or an autoimmune disease. In some embodiments, the autoimmune disease is multiple sclerosis.

It is to be understood that while the disclosure is read in conjunction with the detailed description thereof, the foregoing description and following example are intended to illustrate and not limit the scope of the disclosure, which is defined by the scope of the appended claims. Other aspects, advantages, and modifications are within the scope of the following claims.

### EXAMPLES

### Example 1—Salt Screening of (S)-Mepazine

Initial characterization of raw materials: The free form of (S)-mepazine was liberated from hydrochloride appeared as light purple powder. The physical and thermal properties of this batch of free form were characterized by PLM, XRPD, DSC, TGA and NMR.

X-ray Powder Diffractometer (XRPD): Samples were run on XRPD using below method:
Tube: Cu: K-Alpha (λ=1.54179 Å).
Generator: Voltage: 40 kV; Current: 40 mA.
Scan Scope: 3 to 40 degrees;
Sample rotation speed: 15 rpm.
Scanning rate: 10 deg./min

Differential Scanning calorimetric (DSC): Samples (˜ 1 mg) were tested using a hermetic aluminum pan with pinhole and heated from 25° C. to 250° C. at a rate of 10° C./min under 50 mL/min of N2.

Thermal Gravimetric Analysis (TGA): Samples (3˜ 5 mg) were placed in an open platinum pan and heated from 30° C. to 300° C. or weight %<80% at a rate of 10° C./min under 25 mL/min of N2.

Polarized Light Microscope (PLM): Details of polarized light microscope method used in the tests are mentioned below:
Nikon LV100POL equipped with 5 megapixel CCD.
Physical Lens: 10×/20×.

Dynamic Vapor Sorption (DVS): Samples (˜20 mg) were transferred into a DVS instrument and recorded the weight change with respect to the atmospheric humidity at 25° C. using the following parameters: Equilibrium: dm/dt: 0.002%/min. (for min: 60 min and max: 360 min).

RH (%) measurement step: 10%; RH (%) measurement step scope:40-0-95-0-40.

Results were summarized and are shown in the Table 1 and 3 below:

TABLE 1

| Name | Free form ((S)-Mepazine) | |
|---|---|---|
| Parameter | Method | Result |
| X-ray diffraction | XRPD, 3-40° (2 theta) | High-crystallinity |
| Purity | HPLC | 96.6% |
| Melting onset and | DSC, 10° C./min | 100.6° C., 87.9 J/g |
| enthalpy | | |
| Thermogravimetry | TGA, 10° C./min | 0.32% @ about 85° C. |
| Residual solvent | ¹H-NMR | 0.8% DCM |
| Particle size and | Microscopy | Plate, 20~50 um |
| morphology | | |
| pKa | UV | 9.24 |

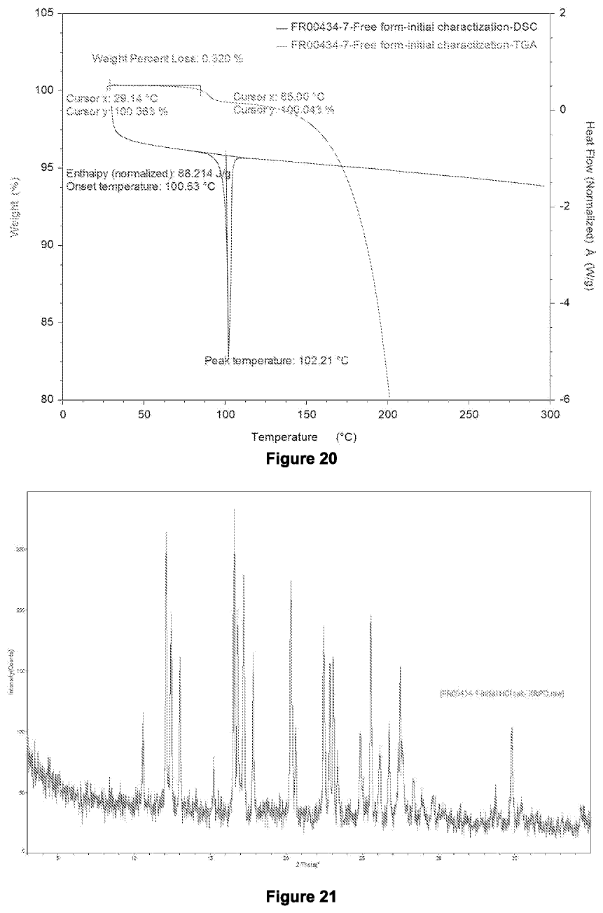

The free form had a high-crystallinity form according to the polarized light microscope photographs and XRPD analysis (Error! Reference source not found. 9). It shows a relative low melting onset of 100.6° ° C. and an enthalpy of 87.9 J/g. The weight loss was 0.32% at around 85° C. (FIG. 20).

Approximate solubility tests of raw material: About 2 mg of free form (S)-mepazine was weighed into 2.0 mL glass vials and then selected solvents were added in the vials stepwise until all solid was dissolved. The experiment was conducted by manual dilution combined with visual observation at 25° C. The total volumes of solvents added were recorded.

The results are listed in Table 2. Based on the solubility of the free form in organic solvents, acetone, ethanol and ethyl acetate were selected for salt screening.

TABLE 2

| Solvent | Solubility (mg/mL) at 25° C. |
|---|---|
| Water | S < 2 |
| Methanol | S > 100 |
| Ethanol | S > 100 |
| 2-Propanol | 33.3 < S < 50 |
| Ethyl acetate | 50 < S < 100 |
| Isopropyl acetate | 50 < S < 100 |
| Acetone | S > 100 |
| Methyl ethyl ketone | S > 100 |
| t-Butyl methyl ether | 25 < S < 33.3 |
| 1,4-Dioxane | S > 100 |
| Tetrahydrofuran | S > 100 |
| Acetonitrile | 25 < S < 33.3 |
| Toluene | S > 100 |
| Heptane | 10 < S < 14.3 |
| Dichloromethane | S > 100 |
| DMF | S > 100 |

Salt screening experiments: 16 counter-ions were selected for salt screening in three solvents ethanol, acetone and ethyl acetate. 30 mg of free form (S)-mepazine was weighted into vials individually, followed by 300 μL solvents were added into vials. For solid counter-ions, 1.0 equivalent (e.q.) of each counter-ion was weighted into 2 ml vials. For liquid acid, 1.0 e.q. counter-ions solutions of corresponding solvents were added to the vials (totally 300 μL). All the vials were placed on the thermo-mixer and heated to 50° C. After keeping at 50° C. for 2 hrs, all the samples were kept stirring at 25° C. for 2 days. Precipitates were collected by centrifugal filtration and analyzed by XRPD. Potential salts were characterized by XRPD, TGA, DSC and NMR. Hygroscopicity is an important property of potential salts, so weight loss investigated by TGA under 92.5% RH condition for 1 week was used to evaluate the hygroscopicity of candidates. Water sorption and desorption behavior were also investigated by DVS at 25° C. through 0(120 min)-95% RH(180 min) humidity, dm/dt was 0.002%. The characterization results are reported in Table 3.

Hemi-fumarate formation: About 30 mg compound was weighted into a 2 ml vial, and then 200 μL Acetone was added into the vial. The sample was placed on the thermo-mixer and kept at 50° C. until the solution became clear. Then 0.5a.q. fumaric acid with 100 μL water and Acetone mixture solution was added into the vial. The sample was kept on the thermo-mixer at 50° ° C. and stirring at 400 rpm. After keeping at 50° ° C. for 2 hrs, the sample was stirred at 25° C. for 24 h.

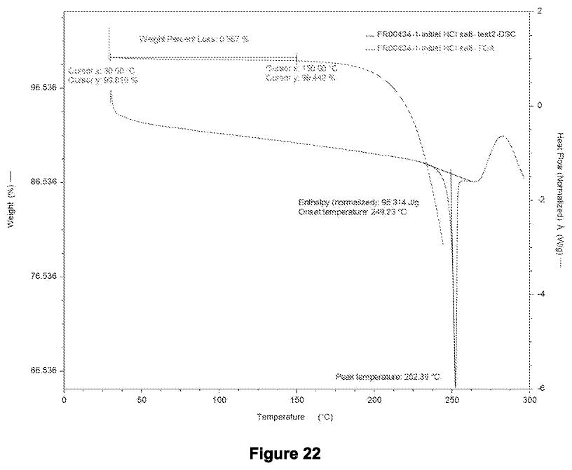

TABLE 3

| | HCl | Sulfate | Phosphate | Tartarate | | Fumarate |
|---|---|---|---|---|---|---|
| Crystallinity | High | Medium | Medium | Medium | Medium | High |
| (By XRPD) | (FIG. 21) | | | Pattern I | Pattern II | (FIG. 3) |
| | | | | (FIG. 9) | (FIG. 11) | |
| Melting | 249.2 | 184.5; 271.7 | 93.3, 231.4 | 149.3 | 95.2; 159.7 | 204.2 |
| onset | (FIG. 22) | | | (FIG. 10) | (FIG. 12) | (FIG. 4) |
| (DSC, ° C.) | | | | | | |
| Enthalpy | D | 48.1; D | 3.2; D | D | 5.4; 66.3 | D |
| (DSC, J/g) | (FIG. 22) | | | (FIG. 10) | (FIG. 12) | (FIG. 4) |
| Weight loss | 0.37% at | 1.6% at | 0.3% at | 0.93% at | 0.59% at | 0.17% at |
| (TGA, %) | 150° C. | 175° C. | 145° C. | 130° C. | 130° C. | 150° C. |
| | (FIG. 22) | | | (FIG. 10) | (FIG. 12) | (FIG. 4) |
| Stoichiometric | 1:0.98 | 1:0.85 | N/A | 1:0.62 | 1:1.5 | 1:0.98 |
| ratio (by | | | | | | |
| ¹H-NMR/IC) | | | | | | |
| Residual | N/A | ND | ND | ND | 0.1% EA | 0.79% |
| solvent | | | | | | Acetone |
| (by ¹H-NMR) | | | | | | |
| 92.5% RH for | Deliqu | Discoloration, | 1.3% at | Deliqu | Deliqu | 0.3% at |
| 1W | | Agglomerate | 145° C. | | | 145° C. |
| (by TGA,%) | | 2.3% at 145° C. | | | | |
| DVS Result | N/A | N/A | 5.18% | N/A | N/A | 1.29% |
| 95% RH | | | | | | |

| | Hemi-fumarate | | Malate | Succinate | Glutamate | Adipate |
|---|---|---|---|---|---|---|
| Crystallinity | Low | Medium | High | High | High | High |
| (By XRPD) | Pattern I | Pattern II | (FIG. 13) | (FIG. 1) | (FIG. 15) | (FIG. |
| | (FIG. 5) | (FIG. 7) | | | | 17) |
| Melting | 148.9; 188.0 | 151.8; 162.0; | 138.0 | 102.5; 164.5 | 98.9; 202.8 | 132.4 |
| onset | (FIG. 6) | 194.9 | (FIG. 14) | (FIG. 2) | (FIG. 16) | (FIG. 18) |
| (DSC, ° C.) | | (FIG. 8) | | | | |
| Enthalpy | D | D | 62.0 | 3.5; D | 10.0; D | 62.7 |
| (DSC, J/g) | (FIG. 6) | (FIG. 8) | (FIG. 14) | (FIG. 2) | (FIG. 16) | (FIG. 18) |
| Weight loss | 0.84% at | 0.47% at | 0.26% at | 0.44% at | 0.65% at | 0.37% at |
| (TGA, %) | 100° C. | 130° C. | 130° C. | 150° C. | 168° C. | 130° C. |
| | (FIG. 6) | (FIG. 8) | (FIG. 14) | (FIG. 2) | (FIG. 16) | (FIG. 18) |
| Stoichiometric | N/A | 1:0.47 | 1:1.07 | 1:1.05 | 1:0.5 | 1:1.18 |
| ratio (by | | | | | | |
| ¹H-NMR/IC) | | | | | | |
| Residual | N/A | 0.5% | 0.67% EA | ND | 0.3% | ND |
| solvent | | Acetone | | | Acetone | |
| (by ¹H-NMR) | | | | | | |
| 92.5% RH for | N/A | N/A | Deliqu | 0.4% at | 2.4% at | 0.6% at |
| 1W | | | | 145° C. | 145° C. | 145° C. |
| (by TGA,%) | | | | | | |
| DVS Result | N/A | N/A | N/A | 1.87% | 16.84% | N/A |
| 95% RH | | | | | | |

Note:
D means that the sample is decomposed before melting point;
Deliqu means deliquescent;
ND means no detectable

Table 4 shows the physico-chemical properties of the mono-fumarate crystalline salt form of (S)-mepazine, mono-succinate crystalline salt form of (S)-mepazine, and the comparative example (i.e., hydrochloride salt form of (S)-mepazine.

TABLE 4

| | Physical form | | |
|---|---|---|---|
| Parameter | Hydrochloride | Mono-fumarate | Mono-succinate |

Crystallinity and thermal properties

| Crystallinity | High | High | High |
|---|---|---|---|
| by XRPD | | | |

Stoichiometry

| | | | |
|---|---|---|---|
| NMR/IC | 1:0.98 | 1:0.99 | 1:1.00 |

Residual solvent

| | | | |
|---|---|---|---|
| NMR | No detectable | 0.56% Acetone | No detectable |

DSC, heating rate [10° C./min]

| Melting | 249.2 | 204.7 | 165.4 |
|---|---|---|---|
| onset (° C.) | | | |
| Melting | Decomposed | Decomposed | Decomposed |
| enthalpy | | | |
| (J/g) | | | |

Themogravimetry [10° C./min]

| | | | |
|---|---|---|---|
| Weight loss | 0.37% at 150° C. | 0.28% at 150° C. | 0.33% at 100° C. |
| in % | | | |

Initial purity [area %]

| | | | |
|---|---|---|---|
| HPLC | 99.9% | 99.7% | 99.7% |

DVS at 25° C. (method: 50-90-0-50% RH) dm/dt = 0.01%

| | Sorption | Desorp | Sorption | Desorp | Sorption | Desorp |
|---|---|---|---|---|---|---|
| | (%) | (%) | (%) | (%) | (%) | (%) |
| 0% | 0.00 | 0.46 | 0.00 | −0.03 | 0.00 | −0.03 |
| 10% | 0.08 | 1.55 | 0.02 | 0.00 | 0.01 | 0.00 |
| 20% | 0.15 | 2.10 | 0.04 | 0.02 | 0.03 | 0.02 |
| 30% | 0.22 | 2.56 | 0.05 | 0.04 | 0.05 | 0.04 |
| 40% | 0.30 | 3.10 | 0.07 | 0.06 | 0.07 | 0.06 |
| 50% | 0.38 | 3.91 | 0.09 | 0.08 | 0.09 | 0.08 |
| 60% | 0.48 | 4.97 | 0.11 | 0.10 | 0.11 | 0.11 |
| 70% | 0.60 | 6.37 | 0.13 | 0.13 | 0.14 | 0.16 |
| 80% | 0.85 | 8.44 | 0.16 | 0.18 | 0.18 | 0.22 |
| 90% | 2.01 | 12.90 | 0.22 | 0.24 | 0.32 | 0.36 |
| 95% | 16.86 | 16.86 | 0.33 | 0.33 | 0.61 | 0.61 |

| | | | |
|---|---|---|---|
| XRPD after | No change | No change | No change |
| DVS cycle | | | |

Morphology

| | | | |
|---|---|---|---|
| PLM | Plate-like | Plate-like | Plate-like |

Particle size

| | | | |
|---|---|---|---|
| PLM | 20~50 um | 10~20 um | 5~10 um |

Solubility at 25° C., target concentration 2 mg/mL

| 24 h | Solubility | Solubility | | Solubility | Solubility | | Solubility | Solubility | |
|---|---|---|---|---|---|---|---|---|---|
| (mg/mL) | (pH) | (pH) | XRPD | (pH) | (pH) | XRPD | (pH) | (pH) | XRPD |
| SGF, pH | >2 (1.79) | >10 (1.67) | N/A | >2 (1.99) | 7.5 (2.60) | NC | >2 (2.04) | 9.3 (3.91) | N/A |
| 2.0 | | | | | | | | | |
| FaSSIF, | >2 (6.47) | >10 (6.23) | N/A | >2 (5.48) | 9.4 (3.63) | N/A | >2 (5.85) | >10 (4.77) | N/A |
| pH 6.5 | | | | | | | | | |
| FeSSIF, | >2 (4.99) | 3.1 (4.88) | N/A | >2 (4.89) | 3.4 (4.52) | N/A | >2 (4.97) | 4.0 (4.76) | N/A |
| pH 5.0 | | | | | | | | | |
| H₂O | >2 (6.69) | >10 (4.48) | N/A | >2 (3.80) | 3.8 (3.49) | NC | >2 (4.94) | >10 (4.49) | N/A |

NC—no change

Methods of scaling up the crystalline salt forms shown below:

Fumarate Crystalline Salt Forms: 303.34 mg free form of (S)-mepazine was weighted into 2 mL Acetone. The sample was kept stirring at 50° C. until it became clear. 123.97 mg fumaric acid with acetone solution was added to the flask (totally 1 mL); (Clear). The solution was held at 50° C. for 2 h, then 20 mg fumarate salt form as seeds were added into the sample; (Clear to Suspension). The suspension was cooled to 25° C. and stirred for 2 days; (Suspension). The suspension was collected by centrifugal filtration and dried in the vacuum oven at 50° C. for 20 hrs resulting in 344 mg dried solids and the yield was 76.9%. (off-white).

Succinate Crystalline Salt Form: 303.68 mg free form of (S)-mepazine was weighted into 2 mL EtOH. The sample was kept stirring at 50° C. until it became clear. 125.89 mg succinic acid with an EtOH and H₂O mixture solution (V:V, 9:1) was added to the flask (totaling 1 mL); (Clear). The solution was held at 50° ° C. for 2 h, then 20 mg succinate crystalline salt form as seeds was added into the sample; (Clear to Suspension). The sample is then cooled to 25° C. and let stir for 2 days; (Suspension). The suspension was collected by centrifugal filtration and dried in the vacuum oven at 50° C. for 20 hrs. 324 mg dried solids were obtained and the yield was 72.0% (off-white).

X-ray Powder Diffraction (XRPD) were run for the succinate salt, the fumarate salt, the hemi-fumarate salt form I, the hemi-fumarate salt form II, the adipate salt, the L-tartrate salt form I, the L-tartrate salt form II, the malate salt form, and the glutamate salt form. The XRPD data for the salt forms are shown in Tables 5-12 below. The XRPD peaks for each XRPD pattern are ranked such that the peaks classified as 1 are defining peaks for the pattern, peaks classified as 2 are less relevant peaks for the pattern, and peaks classified as 3 are the least relevant peaks for the pattern.

X-ray powder diffraction (XRPD) data: samples were run on XRPD using the below method:
Tube: Cu: K-Alpha (λ=1.54179 Å);
Generator: Voltage: 40 kV; Current: 40 mA;
Scan Scope: 2 to 40 degrees;
Sample rotation speed: 15 rpm;
Scanning rate: 10 deg./min or others.

TABLE 5
Succinate Salt Form

| Angle | d Value | Net Intensity | Gross Intensity | Rel. Intensity | Position | Intensity | Classification |
|---|---|---|---|---|---|---|---|
| 3.36 | 26.2733 | 30.308 | 112.599 | 10.40% | 3.36 | 112.599 | 3 |
| 4.062 | 21.7378 | 33.5584 | 96.4597 | 11.50% | 4.062 | 96.4597 | 3 |
| 10.822 | 8.16833 | 109.582 | 146.145 | 37.40% | 10.822 | 146.145 | 1 |
| 12.004 | 7.36712 | 140.737 | 177.549 | 48.10% | 12.004 | 177.549 | 1 |
| 13.448 | 6.57906 | 71.2038 | 105.288 | 24.30% | 13.448 | 105.288 | 2 |
| 14.125 | 6.26506 | 29.7859 | 62.9732 | 10.20% | 14.125 | 62.9732 | 3 |
| 14.419 | 6.13812 | 28.3059 | 61.0206 | 9.70% | 14.419 | 61.0206 | 3 |
| 15.977 | 5.54281 | 83.9343 | 114.765 | 28.70% | 15.977 | 114.765 | 2 |
| 16.823 | 5.26601 | 292.813 | 327.675 | 100.00% | 16.823 | 327.675 | 1 |
| 17.618 | 5.02998 | 127.965 | 164.016 | 43.70% | 17.618 | 164.016 | 1 |
| 18.588 | 4.76966 | 245.415 | 280.34 | 83.80% | 18.588 | 280.34 | 1 |
| 19.332 | 4.58778 | 105.938 | 138.152 | 36.20% | 19.332 | 138.152 | 1 |
| 20.029 | 4.42965 | 77.0361 | 106.702 | 26.30% | 20.029 | 106.702 | 2 |
| 21.389 | 4.15088 | 62.9664 | 92.6247 | 21.50% | 21.389 | 92.6247 | 2 |
| 21.69 | 4.09403 | 40.2502 | 70.3245 | 13.70% | 21.69 | 70.3245 | 3 |
| 23.165 | 3.83662 | 154.706 | 183.947 | 52.80% | 23.165 | 183.947 | 1 |
| 23.673 | 3.75533 | 28.1408 | 56.7246 | 9.60% | 23.673 | 56.7246 | 3 |
| 24.139 | 3.68397 | 25.3365 | 52.9488 | 8.70% | 24.139 | 52.9488 | 3 |
| 24.416 | 3.64268 | 26.8193 | 53.9014 | 9.20% | 24.416 | 53.9014 | 3 |
| 25.174 | 3.53481 | 21.3217 | 48.2127 | 7.30% | 25.174 | 48.2127 | 3 |
| 25.462 | 3.49544 | 40.8696 | 67.3265 | 14.00% | 25.462 | 67.3265 | 3 |
| 27.039 | 3.29509 | 41.0381 | 66.4219 | 14.00% | 27.039 | 66.4219 | 3 |
| 27.504 | 3.24035 | 52.1623 | 78.0786 | 17.80% | 27.504 | 78.0786 | 3 |
| 28.143 | 3.16826 | 21.2447 | 46.4622 | 7.30% | 28.143 | 46.4622 | 3 |
| 28.401 | 3.14002 | 18.1911 | 42.6561 | 6.20% | 28.401 | 42.6561 | 3 |
| 29.064 | 3.06989 | 18.6981 | 39.995 | 6.40% | 29.064 | 39.995 | 3 |
| 29.545 | 3.02097 | 15.3516 | 36.4032 | 5.20% | 29.545 | 36.4032 | 3 |
| 30.903 | 2.89128 | 33.9483 | 52.8647 | 11.60% | 30.903 | 52.8647 | 3 |
| 32.586 | 2.74564 | 22.506 | 41.3402 | 7.70% | 32.586 | 41.3402 | 3 |
| 33.892 | 2.64279 | 16.0403 | 35.9252 | 5.50% | 33.892 | 35.9252 | 3 |

TABLE 6
Fumarate Salt Form

| Angle | d Value | Net Intensity | Gross Intensity | Rel. Intensity | Position | Intensity | Classification |
|---|---|---|---|---|---|---|---|
| 5.503 | 16.0453 | 19.6423 | 68.8802 | 4.70% | 5.503 | 68.8802 | 3 |
| 7.831 | 11.2804 | 47.0829 | 86.906 | 11.30% | 7.831 | 86.906 | 3 |
| 10.292 | 8.5877 | 86.0453 | 126.306 | 20.60% | 10.292 | 126.306 | 2 |
| 11.007 | 8.03203 | 152.171 | 190.174 | 36.50% | 11.007 | 190.174 | 1 |
| 11.338 | 7.79827 | 24.4112 | 60.6716 | 5.90% | 11.338 | 60.6716 | 3 |
| 13.187 | 6.70831 | 67.579 | 102.54 | 16.20% | 13.187 | 102.54 | 3 |
| 13.751 | 6.43466 | 43.7423 | 76.9449 | 10.50% | 13.751 | 76.9449 | 3 |
| 15.661 | 5.65392 | 41.4784 | 75.1131 | 9.90% | 15.661 | 75.1131 | 3 |
| 16.108 | 5.49798 | 187.917 | 223.318 | 45.10% | 16.108 | 223.318 | 1 |
| 16.471 | 5.37766 | 122.079 | 158.206 | 29.30% | 16.471 | 158.206 | 2 |
| 16.768 | 5.28291 | 114.119 | 150.366 | 27.40% | 16.768 | 150.366 | 2 |
| 17.695 | 5.00823 | 417.12 | 454.589 | 100.00% | 17.695 | 454.589 | 1 |
| 18.106 | 4.89565 | 229.03 | 266.386 | 54.90% | 18.106 | 266.386 | 1 |
| 18.241 | 4.85972 | 184.946 | 222.088 | 44.30% | 18.241 | 222.088 | 1 |
| 19.434 | 4.56379 | 38.2805 | 73.0474 | 9.20% | 19.434 | 73.0474 | 3 |
| 19.837 | 4.47214 | 215.11 | 249.957 | 51.60% | 19.837 | 249.957 | 1 |
| 21.095 | 4.2081 | 24.4464 | 60.5469 | 5.90% | 21.095 | 60.5469 | 3 |
| 21.537 | 4.12276 | 109.482 | 146.54 | 26.20% | 21.537 | 146.54 | 2 |
| 22.062 | 4.02575 | 248.517 | 285.488 | 59.60% | 22.062 | 285.488 | 1 |
| 22.234 | 3.99507 | 140.973 | 177.626 | 33.80% | 22.234 | 177.626 | 2 |
| 22.862 | 3.88673 | 204.235 | 239.706 | 49.00% | 22.862 | 239.706 | 1 |
| 23.076 | 3.85123 | 32.7228 | 67.9537 | 7.80% | 23.076 | 67.9537 | 3 |
| 23.584 | 3.76936 | 43.649 | 77.4222 | 10.50% | 23.584 | 77.4222 | 3 |
| 24.323 | 3.65644 | 99.4754 | 131.021 | 23.80% | 24.323 | 131.021 | 2 |
| 24.981 | 3.56165 | 43.91 | 72.723 | 10.50% | 24.981 | 72.723 | 3 |
| 25.915 | 3.43528 | 50.0453 | 76.9349 | 12.00% | 25.915 | 76.9349 | 3 |
| 26.859 | 3.31674 | 85.8138 | 114.293 | 20.60% | 26.859 | 114.293 | 3 |
| 27.188 | 3.27734 | 42.4737 | 70.495 | 10.20% | 27.188 | 70.495 | 3 |
| 27.661 | 3.22231 | 70.8669 | 97.3141 | 17.00% | 27.661 | 97.3141 | 3 |
| 28.858 | 3.09134 | 87.0812 | 111.256 | 20.90% | 28.858 | 111.256 | 3 |
| 29.446 | 3.03095 | 30.2042 | 52.3686 | 7.20% | 29.446 | 52.3686 | 3 |
| 29.654 | 3.01016 | 27.3289 | 48.3826 | 6.60% | 29.654 | 48.3826 | 3 |
| 31.407 | 2.84603 | 103.481 | 124.442 | 24.80% | 31.407 | 124.442 | 3 |
| 32.461 | 2.75601 | 15.1771 | 37.4515 | 3.60% | 32.461 | 37.4515 | 3 |
| 32.725 | 2.73432 | 18.9917 | 42.6751 | 4.60% | 32.725 | 42.6751 | 3 |
| 33.547 | 2.66917 | 11.9426 | 37.8486 | 2.90% | 33.547 | 37.8486 | 3 |
| 34.839 | 2.57309 | 18.0637 | 42.49 | 4.30% | 34.839 | 42.49 | 3 |
| 36.052 | 2.48926 | 27.8296 | 49.3465 | 6.70% | 36.052 | 49.3465 | 3 |

TABLE 7
Adipate Salt Form

| Angle | d Value | Net Intensity | Gross Intensity | Rel. Intensity | Position | Intensity | Classification |
|---|---|---|---|---|---|---|---|
| 8.132 | 10.8641 | 77.6991 | 117.231 | 5.70% | 8.132 | 117.231 | 3 |
| 10.993 | 8.04198 | 116.689 | 155.201 | 8.60% | 10.993 | 155.201 | 3 |
| 13.043 | 6.78223 | 194.315 | 237.526 | 14.30% | 13.043 | 237.526 | 2 |
| 14.169 | 6.24558 | 148.105 | 192.51 | 10.90% | 14.169 | 192.51 | 3 |
| 14.75 | 6.00076 | 539.215 | 581.167 | 39.80% | 14.75 | 581.167 | 1 |
| 15.875 | 5.57799 | 41.1909 | 83.4896 | 3.00% | 15.875 | 83.4896 | 3 |
| 16.333 | 5.42275 | 31.049 | 74.4328 | 2.30% | 16.333 | 74.4328 | 3 |
| 17.399 | 5.09286 | 486.516 | 536.874 | 35.90% | 17.399 | 536.874 | 1 |
| 17.702 | 5.00628 | 1356.16 | 1407.75 | 100.00% | 17.702 | 1407.75 | 1 |
| 17.943 | 4.9396 | 117.496 | 169.496 | 8.70% | 17.943 | 169.496 | 3 |
| 18.694 | 4.74291 | 149.618 | 199.726 | 11.00% | 18.694 | 199.726 | 3 |
| 19.279 | 4.60023 | 224.087 | 272.053 | 16.50% | 19.279 | 272.053 | 2 |
| 20.463 | 4.33658 | 92.0051 | 138.643 | 6.80% | 20.463 | 138.643 | 3 |
| 21.325 | 4.16324 | 132.267 | 181.791 | 9.80% | 21.325 | 181.791 | 3 |
| 21.629 | 4.10533 | 963.464 | 1013.72 | 71.00% | 21.629 | 1013.72 | 1 |
| 22.031 | 4.03142 | 81.4196 | 131.431 | 6.00% | 22.031 | 131.431 | 3 |
| 22.816 | 3.89439 | 15.6331 | 65.6383 | 1.20% | 22.816 | 65.6383 | 3 |
| 23.161 | 3.83722 | 20.7803 | 73.8263 | 1.50% | 23.161 | 73.8263 | 3 |
| 23.655 | 3.75821 | 165.889 | 221.525 | 12.20% | 23.655 | 221.525 | 3 |
| 23.864 | 3.72576 | 288.564 | 344.67 | 21.30% | 23.864 | 344.67 | 2 |
| 23.945 | 3.71332 | 241.758 | 297.946 | 17.80% | 23.945 | 297.946 | 2 |
| 24.817 | 3.58479 | 99.9625 | 157.66 | 7.40% | 24.817 | 157.66 | 3 |
| 25.264 | 3.52231 | 182.59 | 239.658 | 13.50% | 25.264 | 239.658 | 3 |
| 25.406 | 3.50306 | 218.963 | 275.473 | 16.10% | 25.406 | 275.473 | 3 |
| 25.942 | 3.43185 | 540.195 | 593.046 | 39.80% | 25.942 | 593.046 | 1 |
| 26.642 | 3.34326 | 56.0402 | 100.417 | 4.10% | 26.642 | 100.417 | 3 |
| 26.858 | 3.31679 | 18.2202 | 59.1253 | 1.30% | 26.858 | 59.1253 | 3 |
| 28.493 | 3.13007 | 133.183 | 173.665 | 9.80% | 28.493 | 173.665 | 3 |
| 28.762 | 3.10144 | 19.6273 | 59.4622 | 1.40% | 28.762 | 59.4622 | 3 |
| 29.393 | 3.03631 | 34.7997 | 73.1423 | 2.60% | 29.393 | 73.1423 | 3 |
| 29.629 | 3.01258 | 91.3084 | 128.625 | 6.70% | 29.629 | 128.625 | 3 |
| 31.303 | 2.85522 | 97.6143 | 132.951 | 7.20% | 31.303 | 132.951 | 3 |
| 31.949 | 2.79895 | 24.2829 | 57.2108 | 1.80% | 31.949 | 57.2108 | 3 |
| 32.105 | 2.78569 | 28.0873 | 61.1476 | 2.10% | 32.105 | 61.1476 | 3 |
| 32.75 | 2.73232 | 12.2216 | 44.9205 | 0.90% | 32.75 | 44.9205 | 3 |
| 35.759 | 2.50899 | 18.4187 | 50.1333 | 1.40% | 35.759 | 50.1333 | 3 |
| 36.042 | 2.48997 | 26.3424 | 57.1586 | 1.90% | 36.042 | 57.1586 | 3 |
| 37.327 | 2.4071 | 13.3547 | 47.2931 | 1.00% | 37.327 | 47.2931 | 3 |
| 37.92 | 2.37081 | 19.0014 | 55.7007 | 1.40% | 37.92 | 55.7007 | 3 |
| 39.023 | 2.30632 | 28.7347 | 63.2291 | 2.10% | 39.023 | 63.2291 | 3 |
| 39.229 | 2.2947 | 24.6405 | 57.5718 | 1.80% | 39.229 | 57.5718 | 3 |

TABLE 8A
Hemi-Fumarate Salt Form I

| Angle | d Value | Net Intensity | Gross Intensity | Rel. Intensity | Position | Intensity | Classification |
|---|---|---|---|---|---|---|---|
| 7.834 | 11.2767 | 18.8799 | 57.8667 | 15.70% | 7.834 | 57.8667 | 3 |
| 10.144 | 8.7128 | 91.6391 | 132.138 | 76.30% | 10.144 | 132.138 | 1 |
| 10.992 | 8.04254 | 55.3886 | 96.3613 | 46.10% | 10.992 | 96.3613 | 1 |
| 11.797 | 7.49561 | 24.6056 | 64.2195 | 20.50% | 11.797 | 64.2195 | 3 |
| 11.993 | 7.37382 | 120.045 | 159.048 | 100.00% | 11.993 | 159.048 | 1 |
| 13.158 | 6.72299 | 22.3968 | 58.7825 | 18.70% | 13.158 | 58.7825 | 3 |
| 15.473 | 5.72218 | 47.772 | 83.4164 | 39.80% | 15.473 | 83.4164 | 2 |
| 16.089 | 5.50456 | 35.0473 | 69.4295 | 29.20% | 16.089 | 69.4295 | 3 |
| 16.456 | 5.38247 | 42.9823 | 76.0921 | 35.80% | 16.456 | 76.0921 | 2 |
| 16.767 | 5.28342 | 42.9987 | 75.5827 | 35.80% | 16.767 | 75.5827 | 2 |
| 17.681 | 5.01222 | 108.037 | 144.11 | 90.00% | 17.681 | 144.11 | 1 |
| 18.045 | 4.91194 | 63.7247 | 101.483 | 53.10% | 18.045 | 101.483 | 1 |
| 18.221 | 4.86484 | 53.5938 | 92.0308 | 44.60% | 18.221 | 92.0308 | 1 |
| 18.684 | 4.74529 | 15.5894 | 55.3856 | 13.00% | 18.684 | 55.3856 | 3 |
| 19.814 | 4.47717 | 45.6983 | 86.229 | 38.10% | 19.814 | 86.229 | 2 |
| 22.044 | 4.02909 | 59.8848 | 93.3464 | 49.90% | 22.044 | 93.3464 | 1 |
| 22.227 | 3.99628 | 41.3751 | 74.8346 | 34.50% | 22.227 | 74.8346 | 2 |
| 22.87 | 3.88543 | 22.3783 | 56.7236 | 18.60% | 22.87 | 56.7236 | 3 |
| 23.456 | 3.78956 | 27.3999 | 62.5471 | 22.80% | 23.456 | 62.5471 | 3 |
| 24.147 | 3.68265 | 35.6198 | 70.4437 | 29.70% | 24.147 | 70.4437 | 3 |
| 24.243 | 3.66842 | 39.0548 | 73.727 | 32.50% | 24.243 | 73.727 | 2 |
| 24.961 | 3.56449 | 15.1517 | 47.8399 | 12.60% | 24.961 | 47.8399 | 3 |
| 25.804 | 3.44989 | 0.85922 | 31.6215 | 0.70% | 25.804 | 31.6215 | 3 |
| 26.828 | 3.32043 | 42.4414 | 70.3423 | 35.40% | 26.828 | 70.3423 | 3 |
| 27.617 | 3.22736 | 15.9478 | 43.3911 | 13.30% | 27.617 | 43.3911 | 3 |
| 28.848 | 3.09238 | 19.4054 | 44.5167 | 16.20% | 28.848 | 44.5167 | 3 |
| 30 | 2.97625 | 21.303 | 44.8138 | 17.70% | 30 | 44.8138 | 3 |
| 31.398 | 2.84684 | 20.5537 | 43.3047 | 17.10% | 31.398 | 43.3047 | 3 |

TABLE 8B
Hemi-fumarate Salt Form II

| | | Net | Gross | Rel. | | | |
|---|---|---|---|---|---|---|---|
| Angle | d Value | Intensity | Intensity | Intensity | Position | Intensity | Classification |
| 5.859 | 15.0722 | 14.6724 | 58.2313 | 4.70% | 5.859 | 58.2313 | 3 |
| 10.068 | 8.77853 | 21.0314 | 58.061 | 6.70% | 10.068 | 58.061 | 3 |
| 11.648 | 7.59126 | 268.271 | 309.438 | 86.00% | 11.648 | 309.438 | 1 |
| 12.857 | 6.88 | 93.2741 | 134.961 | 29.90% | 12.857 | 134.961 | 2 |
| 13.411 | 6.5968 | 110.743 | 150.536 | 35.50% | 13.411 | 150.536 | 1 |
| 13.886 | 6.3723 | 20.756 | 58.9673 | 6.70% | 13.886 | 58.9673 | 3 |
| 14.225 | 6.22112 | 29.6409 | 68.0804 | 9.50% | 14.225 | 68.0804 | 3 |
| 14.662 | 6.03686 | 49.6101 | 87.6043 | 15.90% | 14.662 | 87.6043 | 3 |
| 15.105 | 5.86069 | 23.4168 | 60.9734 | 7.50% | 15.105 | 60.9734 | 3 |
| 15.449 | 5.73088 | 42.4315 | 79.6735 | 13.60% | 15.449 | 79.6735 | 3 |
| 16.089 | 5.50445 | 65.6799 | 102.349 | 21.10% | 16.089 | 102.349 | 2 |
| 16.656 | 5.31827 | 312.018 | 350.084 | 100.00% | 16.656 | 350.084 | 1 |
| 16.914 | 5.23783 | 23.3784 | 61.617 | 7.50% | 16.914 | 61.617 | 3 |
| 17.485 | 5.06792 | 290.24 | 327.825 | 93.00% | 17.485 | 327.825 | 1 |
| 17.963 | 4.93417 | 16.7971 | 53.3835 | 5.40% | 17.963 | 53.3835 | 3 |
| 20.136 | 4.40642 | 115.003 | 150.877 | 36.90% | 20.136 | 150.877 | 1 |
| 20.626 | 4.30283 | 89.0581 | 126.087 | 28.50% | 20.626 | 126.087 | 2 |
| 21.096 | 4.20788 | 93.6136 | 130.766 | 30.00% | 21.096 | 130.766 | 2 |
| 21.516 | 4.12676 | 56.9312 | 93.3808 | 18.20% | 21.516 | 93.3808 | 3 |
| 22.631 | 3.92587 | 25.4725 | 66.1522 | 8.20% | 22.631 | 66.1522 | 3 |
| 22.876 | 3.88436 | 50.152 | 92.3366 | 16.10% | 22.876 | 92.3366 | 3 |
| 23.975 | 3.70873 | 108.41 | 154.122 | 34.70% | 23.975 | 154.122 | 2 |
| 24.671 | 3.60566 | 148.192 | 193.412 | 47.50% | 24.671 | 193.412 | 1 |
| 24.891 | 3.57424 | 26.3529 | 70.9766 | 8.40% | 24.891 | 70.9766 | 3 |
| 25.505 | 3.48961 | 90.8621 | 134.014 | 29.10% | 25.505 | 134.014 | 2 |
| 26.489 | 3.36217 | 135.46 | 174.801 | 43.40% | 26.489 | 174.801 | 2 |
| 27.76 | 3.21104 | 27.5878 | 62.6651 | 8.80% | 27.76 | 62.6651 | 3 |
| 28.574 | 3.12146 | 43.8829 | 79.523 | 14.10% | 28.574 | 79.523 | 3 |
| 29.267 | 3.04907 | 17.1957 | 51.4964 | 5.50% | 29.267 | 51.4964 | 3 |
| 30.133 | 2.96339 | 15.3472 | 46.2143 | 4.90% | 30.133 | 46.2143 | 3 |
| 31.167 | 2.86734 | 27.1632 | 55.7478 | 8.70% | 31.167 | 55.7478 | 3 |
| 36.963 | 2.43 | 24.2201 | 50.5231 | 7.80% | 36.963 | 50.5231 | 3 |
| 39.088 | 2.3026 | 10.6468 | 36.8409 | 3.40% | 39.088 | 36.8409 | 3 |

TABLE 9
L-tartrate Salt Form I

| Angle | d Value | Net Intensity | Gross Intensity | Rel. Intensity | Position | Intensity | Classification |
|---|---|---|---|---|---|---|---|
| 3.114 | 28.3521 | 30.8425 | 145.689 | 15.00% | 3.114 | 145.689 | 3 |
| 3.868 | 22.8221 | 18.4406 | 85.2214 | 9.00% | 3.868 | 85.2214 | 3 |
| 5.198 | 16.988 | 30.0044 | 78.7333 | 14.60% | 5.198 | 78.7333 | 3 |
| 11.326 | 7.80602 | 47.7295 | 86.6589 | 23.20% | 11.326 | 86.6589 | 2 |
| 14.029 | 6.30775 | 34.9775 | 72.0965 | 17.00% | 14.029 | 72.0965 | 3 |
| 14.503 | 6.10278 | 115.113 | 152.334 | 55.90% | 14.503 | 152.334 | 1 |
| 15.643 | 5.66023 | 206.011 | 245.142 | 100.00% | 15.643 | 245.142 | 1 |
| 17.513 | 5.05998 | 182.058 | 223.991 | 88.40% | 17.513 | 223.991 | 1 |
| 18.644 | 4.75542 | 70.9175 | 114.607 | 34.40% | 18.644 | 114.607 | 2 |
| 19.545 | 4.53822 | 48.0208 | 92.0971 | 23.30% | 19.545 | 92.0971 | 2 |
| 20.361 | 4.35806 | 92.6464 | 137.155 | 45.00% | 20.361 | 137.155 | 1 |
| 20.886 | 4.24968 | 33.6417 | 77.6882 | 16.30% | 20.886 | 77.6882 | 3 |
| 22.482 | 3.95151 | 25.3618 | 63.3781 | 12.30% | 22.482 | 63.3781 | 3 |
| 22.785 | 3.89974 | 89.5923 | 128.071 | 43.50% | 22.785 | 128.071 | 1 |
| 23.466 | 3.78795 | 19.9494 | 58.2618 | 9.70% | 23.466 | 58.2618 | 3 |
| 23.978 | 3.70833 | 61.2349 | 99.0769 | 29.70% | 23.978 | 99.0769 | 2 |
| 24.713 | 3.5997 | 53.3403 | 89.4705 | 25.90% | 24.713 | 89.4705 | 2 |
| 26.225 | 3.39549 | 31.2071 | 66.3998 | 15.10% | 26.225 | 66.3998 | 3 |
| 26.805 | 3.32332 | 13.8879 | 48.1768 | 6.70% | 26.805 | 48.1768 | 3 |
| 28.131 | 3.16951 | 16.2361 | 44.6688 | 7.90% | 28.131 | 44.6688 | 3 |
| 28.815 | 3.09586 | 18.7986 | 45.2866 | 9.10% | 28.815 | 45.2866 | 3 |
| 31.244 | 2.86048 | 29.8973 | 56.3696 | 14.50% | 31.244 | 56.3696 | 3 |
| 35.447 | 2.53037 | 16.6806 | 38.8899 | 8.10% | 35.447 | 38.8899 | 3 |

TABLE 10
L-tartrate Salt Form II

| Angle | d Value | Net Intensity | Gross Intensity | Rel. Intensity | Position | Intensity | Classification |
|---|---|---|---|---|---|---|---|
| 10.387 | 8.50956 | 40.1925 | 85.9161 | 12.80% | 10.387 | 85.9161 | 3 |
| 10.862 | 8.13887 | 29.4682 | 74.7854 | 9.40% | 10.862 | 74.7854 | 3 |
| 11.641 | 7.59606 | 114.922 | 157.45 | 36.70% | 11.641 | 157.45 | 1 |
| 13.493 | 6.55683 | 41.1487 | 87.3296 | 13.20% | 13.493 | 87.3296 | 3 |
| 13.845 | 6.39103 | 16.2387 | 63.5244 | 5.20% | 13.845 | 63.5244 | 3 |
| 14.316 | 6.18179 | 87.8111 | 135.734 | 28.10% | 14.316 | 135.734 | 3 |
| 14.646 | 6.04321 | 226.883 | 274.679 | 72.50% | 14.646 | 274.679 | 1 |
| 16.903 | 5.24108 | 32.2773 | 76.6606 | 10.30% | 16.903 | 76.6606 | 3 |
| 17.592 | 5.03734 | 15.723 | 58.9652 | 5.00% | 17.592 | 58.9652 | 3 |
| 18.653 | 4.75328 | 53.1084 | 102.332 | 17.00% | 18.653 | 102.332 | 3 |
| 18.861 | 4.70123 | 164.82 | 215.298 | 52.70% | 18.861 | 215.298 | 1 |
| 19.183 | 4.62313 | 51.7174 | 103.762 | 16.50% | 19.183 | 103.762 | 3 |
| 19.58 | 4.53007 | 33.9553 | 87.3168 | 10.90% | 19.58 | 87.3168 | 3 |
| 20.225 | 4.38713 | 110.704 | 164.78 | 35.40% | 20.225 | 164.78 | 1 |
| 20.757 | 4.27595 | 312.836 | 366.166 | 100.00% | 20.757 | 366.166 | 1 |
| 20.937 | 4.23945 | 66.2851 | 119.083 | 21.20% | 20.937 | 119.083 | 3 |
| 21.222 | 4.18331 | 34.2941 | 85.9668 | 11.00% | 21.222 | 85.9668 | 3 |
| 21.491 | 4.13141 | 28.0677 | 78.346 | 9.00% | 21.491 | 78.346 | 3 |
| 21.706 | 4.09098 | 31.9966 | 80.9385 | 10.20% | 21.706 | 80.9385 | 3 |
| 22.532 | 3.94285 | 27.586 | 70.7574 | 8.80% | 22.532 | 70.7574 | 3 |
| 23.405 | 3.79783 | 25.6415 | 66.979 | 8.20% | 23.405 | 66.979 | 3 |
| 23.675 | 3.75505 | 41.8018 | 84.3347 | 13.40% | 23.675 | 84.3347 | 3 |
| 23.849 | 3.7281 | 63.6776 | 106.81 | 20.40% | 23.849 | 106.81 | 3 |
| 25.098 | 3.54533 | 100.78 | 144.352 | 32.20% | 25.098 | 144.352 | 2 |
| 26.437 | 3.36866 | 66.2141 | 105.811 | 21.20% | 26.437 | 105.811 | 3 |
| 27.832 | 3.20297 | 35.9608 | 69.9407 | 11.50% | 27.832 | 69.9407 | 3 |
| 29.256 | 3.05023 | 107.442 | 138.533 | 34.30% | 29.256 | 138.533 | 2 |
| 29.776 | 2.99805 | 112.891 | 142.549 | 36.10% | 29.776 | 142.549 | 2 |
| 31.512 | 2.83675 | 19.3299 | 49.8895 | 6.20% | 31.512 | 49.8895 | 3 |
| 32.06 | 2.78956 | 92.1575 | 122.856 | 29.50% | 32.06 | 122.856 | 3 |
| 33.467 | 2.67538 | 32.2418 | 62.1211 | 10.30% | 33.467 | 62.1211 | 3 |
| 34.074 | 2.62908 | 13.4328 | 41.6913 | 4.30% | 34.074 | 41.6913 | 3 |
| 35.4 | 2.53362 | 54.2794 | 81.3586 | 17.40% | 35.4 | 81.3586 | 3 |
| 35.864 | 2.50191 | 140.74 | 167.485 | 45.00% | 35.864 | 167.485 | 2 |
| 36.743 | 2.44402 | 98.5846 | 122.128 | 31.50% | 36.743 | 122.128 | 2 |
| 37.474 | 2.39802 | 63.9069 | 86.9877 | 20.40% | 37.474 | 86.9877 | 3 |
| 38.562 | 2.33285 | 29.0136 | 54.7551 | 9.30% | 38.562 | 54.7551 | 3 |

TABLE 11
Malate Salt Form

| Angle | d Value | Net Intensity | Gross Intensity | Rel. Intensity | Position | Intensity | Classification |
|---|---|---|---|---|---|---|---|
| 10.764 | 8.21266 | 20.9472 | 50.1831 | 11.20% | 10.764 | 50.1831 | 3 |
| 11.763 | 7.51721 | 47.2745 | 77.4956 | 25.20% | 11.763 | 77.4956 | 2 |
| 13.376 | 6.61404 | 55.0786 | 84.5155 | 29.40% | 13.376 | 84.5155 | 2 |
| 14.292 | 6.19204 | 36.6331 | 67.3914 | 19.60% | 14.292 | 67.3914 | 3 |
| 15.896 | 5.57097 | 32.4323 | 61.2087 | 17.30% | 15.896 | 61.2087 | 3 |
| 16.908 | 5.23966 | 134.013 | 164.902 | 71.60% | 16.908 | 164.902 | 1 |
| 17.568 | 5.04427 | 98.1127 | 130.263 | 52.40% | 17.568 | 130.263 | 1 |
| 18.321 | 4.83859 | 129.445 | 161.768 | 69.10% | 18.321 | 161.768 | 1 |
| 19.21 | 4.61659 | 111.999 | 144 | 59.80% | 19.21 | 144 | 1 |
| 19.796 | 4.48118 | 61.3945 | 92.0294 | 32.80% | 19.796 | 92.0294 | 2 |
| 21.288 | 4.17038 | 23.9569 | 57.4094 | 12.80% | 21.288 | 57.4094 | 3 |
| 21.645 | 4.10247 | 37.3551 | 71.26 | 19.90% | 21.645 | 71.26 | 3 |
| 22.967 | 3.86917 | 187.291 | 219.184 | 100.00% | 22.967 | 219.184 | 1 |
| 24.195 | 3.67551 | 11.8055 | 43.651 | 6.30% | 24.195 | 43.651 | 3 |
| 24.896 | 3.57359 | 37.23 | 69.1149 | 19.90% | 24.896 | 69.1149 | 3 |
| 25.499 | 3.4905 | 16.0641 | 46.8548 | 8.60% | 25.499 | 46.8548 | 3 |
| 25.711 | 3.46218 | 10.5657 | 40.6841 | 5.60% | 25.711 | 40.6841 | 3 |
| 26.661 | 3.34084 | 33.4075 | 62.1101 | 17.80% | 26.661 | 62.1101 | 3 |
| 27.559 | 3.23401 | 68.2521 | 96.1528 | 36.40% | 27.559 | 96.1528 | 2 |
| 27.898 | 3.19547 | 22.517 | 50.5708 | 12.00% | 27.898 | 50.5708 | 3 |
| 28.236 | 3.15801 | 23.5724 | 51.6325 | 12.60% | 28.236 | 51.6325 | 3 |
| 28.726 | 3.10527 | 26.9799 | 54.3768 | 14.40% | 28.726 | 54.3768 | 3 |
| 29.817 | 2.99409 | 9.98539 | 32.9353 | 5.30% | 29.817 | 32.9353 | 3 |
| 31.388 | 2.8477 | 11.8834 | 34.3045 | 6.30% | 31.388 | 34.3045 | 3 |
| 32.167 | 2.78045 | 18.5512 | 41.5696 | 9.90% | 32.167 | 41.5696 | 3 |
| 35.465 | 2.52911 | 16.8399 | 38.6117 | 9.00% | 35.465 | 38.6117 | 3 |
| 36.672 | 2.44861 | 16.1646 | 35.3544 | 8.60% | 36.672 | 35.3544 | 3 |
| 39.449 | 2.28239 | 10.7041 | 30.559 | 5.70% | 39.449 | 30.559 | 3 |
| 39.557 | 2.27639 | 8.87324 | 28.827 | 4.70% | 39.557 | 28.827 | 3 |

TABLE 12
Glutamate Salt Form

| Angle | d Value | Net Intensity | Gross Intensity | Rel. Intensity | Position | Intensity | Classification |
|---|---|---|---|---|---|---|---|
| 10.248 | 8.62515 | 43.5752 | 85.4835 | 12.20% | 10.248 | 85.4835 | 3 |
| 13.76 | 6.43023 | 41.9033 | 80.2615 | 11.70% | 13.76 | 80.2615 | 3 |
| 17.993 | 4.92588 | 46.5504 | 83.0625 | 13.00% | 17.993 | 83.0625 | 3 |
| 20.072 | 4.42022 | 78.4154 | 119.556 | 21.90% | 20.072 | 119.556 | 2 |
| 20.578 | 4.31277 | 75.5267 | 116.6 | 21.10% | 20.578 | 116.6 | 2 |
| 21.505 | 4.12884 | 357.468 | 398.489 | 100.00% | 21.505 | 398.489 | 1 |
| 22.127 | 4.01422 | 278.319 | 317.956 | 77.90% | 22.127 | 317.956 | 1 |
| 23.194 | 3.83176 | 51.1501 | 89.5562 | 14.30% | 23.194 | 89.5562 | 3 |
| 23.88 | 3.72321 | 87.7427 | 126.94 | 24.50% | 23.88 | 126.94 | 2 |
| 24.289 | 3.66149 | 25.4696 | 64.0724 | 7.10% | 24.289 | 64.0724 | 3 |
| 25.701 | 3.46344 | 289.363 | 326.675 | 80.90% | 25.701 | 326.675 | 1 |
| 26.227 | 3.39513 | 178.878 | 216.108 | 50.00% | 26.227 | 216.108 | 1 |
| 26.518 | 3.35861 | 26.2113 | 62.8309 | 7.30% | 26.518 | 62.8309 | 3 |
| 27.729 | 3.21463 | 70.676 | 101.623 | 19.80% | 27.729 | 101.623 | 3 |
| 28.904 | 3.0865 | 31.6766 | 59.9214 | 8.90% | 28.904 | 59.9214 | 3 |
| 30.09 | 2.96747 | 114.674 | 142.263 | 32.10% | 30.09 | 142.263 | 1 |
| 31.097 | 2.87365 | 242.003 | 268.075 | 67.70% | 31.097 | 268.075 | 1 |
| 31.538 | 2.83445 | 48.274 | 72.1599 | 13.50% | 31.538 | 72.1599 | 3 |
| 32.82 | 2.72664 | 25.4374 | 50.9571 | 7.10% | 32.82 | 50.9571 | 3 |
| 33.134 | 2.70154 | 33.4336 | 59.7199 | 9.40% | 33.134 | 59.7199 | 3 |
| 33.802 | 2.64967 | 61.5441 | 87.9036 | 17.20% | 33.802 | 87.9036 | 3 |
| 34.864 | 2.5713 | 36.6135 | 61.4648 | 10.20% | 34.864 | 61.4648 | 3 |
| 35.774 | 2.50794 | 51.3944 | 76.0232 | 14.40% | 35.774 | 76.0232 | 3 |
| 36.388 | 2.46704 | 32.5977 | 55.8981 | 9.10% | 36.388 | 55.8981 | 3 |
| 38.079 | 2.36127 | 35.6842 | 58.4598 | 10.00% | 38.079 | 58.4598 | 3 |
| 38.714 | 2.324 | 17.1155 | 38.9675 | 4.80% | 38.714 | 38.9675 | 3 |
| 39.354 | 2.28765 | 8.55034 | 29.8912 | 2.40% | 39.354 | 29.8912 | 3 |

### Comparative Crystalline Salt Forms

### Hydrochloride Salt Crystalline Form of (S)-Mepazine

The hydrochloride salt crystalline form is an anhydrate with high crystallinity and exhibits as plate-like particle morphology. It had a melting onset at 249.2° C. along with decomposition. Weight loss was ˜1% at around 180° C. Hydrochloride was sensitive to high humidity, where it absorbed 0.85% moisture at 80% RH at 25° C., but 16.86% moisture was observed at up to 95% RH at 25° C. Agglomeration was observed after DVS test without form change after two sorption/desorption cycles. However, it is deliquescent after storage at 92.5% RH condition for 1 week.

The hydrochloride salt crystalline form polymorphic behavior was investigated by equilibration, evaporation, anti-solvent precipitation and crystallization from hot saturated solutions. No polymorph was observed in this polymorphism screening study. No form change was observed for hydrochloride upon compression. After grinding and wet granulation with ethanol and water, its form also remained unchanged.

The hydrochloride salt crystalline form is a developable risk due to its high hygroscopicity at high humidity condition.

### Example 2—Development of the Succinate Crystalline Salt Form

### Succinate Crystalline Salt Form of (S)-Mepazine

Succinate crystalline salt form of (S)-mepazine was found to be an anhydrate in high crystallinity with plate-like particles and had a melting onset at 165.6° C. along with decomposition. The weight loss before melting was around 0.41%. The stoichiometry of succinate was 1:0.99 and the residual solvent of ethanol was 0.33%. It was slightly hygroscopic, absorbing 1.65% moisture up to 95% RH at 25° C. No form change was observed after DVS measurement. No form change was observed for succinate upon compression. After grinding and wet granulation with ethanol and water, its form also remained unchanged.

### X-Ray Powder Diffraction (XRPD)

X-ray powder diffraction (XRPD) data: samples were run on XRPD using the below method:
Tube: Cu: K-Alpha (λ=1.54179 Å);
Generator: Voltage: 40 kV; Current: 40 mA;
Scan Scope: 2 to 40 degrees;
Sample rotation speed: 15 rpm;
Scanning rate: 10 deg./min or others.
   Differential Scanning calorimetry (DSC)

Differential scanning calorimetry (DSC) analysis: samples (1˜ 2 mg) were tested using a hermetic aluminum pan with pinhole and heated from 25° C. to 250° C. at a rate of 10° C./min under 50 mL/min of N2.

### Thermal Gravimetric Analysis (TGA)

Thermal gravimetric analysis (TGA): Samples (3˜5 mg) were placed in an open platinum pan and heated from 30° C. to 300° C. or weight %<80% at a rate of 10° C./min under 25 mL/min of N2.

Succinate crystalline salt form: 5547.7 mg free form of (S)-Mepazine was weighed into a 100 mL flask and 40 mL EtOH was added into the flask. The sample was kept stirring at 50° C. until it became clear. Then 2309.1 mg succinic acid with EtOH and H₂O mixture solution (V:V, 15:1) was added to the flask (totally 16 mL). After keeping at 50° C. for 30 minutes, the solution was cooled gradually to 25° C. in a period of 50 min. When the temperature dropped to 40° C., 80.17 mg succinate was added into the flask. At the same time, solids were precipitated from clear solution. After stirring for 24 h at 25° C., the suspension was filtered and dried under vacuum at 45° C. for 50 h. The weight of dried succinate solids was 6.06 g and yield was 76.4%.

Water sorption and desorption behavior were investigated by DVS at 25° C. through 40-0-95-0-40% RH humidity cycles, dm/dt was 0.002%. Succinate Form A (batch No. FR00434-15-succinate-scale up) was slightly hygroscopic. DVS study showed that succinate absorbed about 1.648% water by weight when over the humidity range from 0% to 95% at 25° C. Its crystal modification remained unchanged after two sorption/desorption cycles.

Only one succinate salt polymorph was found despite testing for different polymorph by characterizing the solubility in various solvents, equilibration with various solvents and solvent mixtures, slow evaporation of various solvents, crystallization from hot saturated solutions by fast and slow cooling, precipitation by addition of anti-solvent, grinding simulation, and granulation simulation. Solubility of the succinate salt was tested in water, methanol, ethanol, 2-propanol, ethyl acetate, isopropyl acetate, acetone, methyl ethyl ketone, t-butyl methyl ether, 1,4-dioxane, tetrahydrofuran, acetonitrile, toluene, heptane, and dichloromethane.

Approximate solubility at 25° C. and 50° C.: About 2 mg of drug substance was weighted and dissolved with minimal amount of solvent to determine solubility at 25° C. and 50° C. The experiments were performed by manual dilution combined with visual observation. Solubility of the succinate salt was tested in water, methanol, ethanol, 2-propanol, ethyl acetate, isopropyl acetate, acetone, methyl ethyl ketone, t-butyl methyl ether, 1,4-dioxane, tetrahydrofuran, acetonitrile, toluene, heptane, and dichloromethane. The solid form was evaluated and the degree of crystallinity, if it was crystalline, by XRPD. No differences were observed from the succinate salt crystallized form as seen in FIG. 1.

Equilibration with solvents at 25° C. for 1 week and 50° C. for 1 week: About 30 mg of drug substance was equilibrated in 0.2-0.5 mL of solvent at 25° C. or 50° C. for 2 weeks with a stirring plate or an eppendorf shaker. If some samples were clear, drug substance is increased to 50 mg. The suspension that was obtained was filtered, if applicable. The solid part (wet cake) was investigated by XRPD. Equilibration of the succinate salt was tested in water, methanol, ethanol, 2-propanol, ethyl acetate, isopropyl acetate, acetone, methyl ethyl ketone, t-butyl methyl ether, 1,4-dioxane, tetrahydrofuran, acetonitrile, toluene, heptane, dichloromethane, dimethylformamide, 50:50 (v:v) acetone/water, 50:50 (v:v) methanol/water, 25:75 (v:v) ethanol/water, 50:50 (v:v) ethanol/water, 90:10 (v:v) ethanol/water, and 95:5 (v:v) ethanol/water. The solid form was evaluated and the degree of crystallinity, if it was crystalline, by XRPD. No differences were observed from the succinate salt crystallized form as seen in FIG. 1.

Slow evaporation at ambient temperature: About 30 mg of drug substance was dissolved in a solvent until it became clear, and the upper solution allowed to evaporate slowly at ambient temperature. Residual solid part was investigated by XRPD. The slow evaporation at ambient temperature of the succinate salt was tested in water, methanol, ethanol, 2-propanol, ethyl acetate, isopropyl acetate, acetone, methyl ethyl ketone, t-butyl methyl ether, 1,4-dioxane, tetrahydrofuran, acetonitrile, toluene, heptane, dichloromethane, 50:50 (v:v) acetone/water, 50:50 (v:v) methanol/water, 75:25 (v:v) ethanol/water, 50:50 (v:v) ethanol/water. The solid form was evaluated and the degree of crystallinity, if it was crystalline, by XRPD. No differences were observed from the succinate salt crystallized form as seen in FIG. 1.

Crystallization from hot saturated solutions by fast and slow cooling: Approximately 30 mg of drug substance was dissolved in the minimal amount of selected solvents at 60° C. The obtained solutions were put into an ice bath for fast cooling or applied with cooling rate of 0.1° C./min for slow cooling. Precipitates were collected by filtration and investigated as described in the equilibration with solvents above. If no precipitation was obtained at 0° C., the solutions shall be put in −20° C. freezer for crystallization. The crystallization from hot saturated solutions of the succinate salt was tested in water, methanol, ethanol, 2-propanol, ethyl acetate, isopropyl acetate, acetone, methyl ethyl ketone, t-butyl methyl ether, 1,4-dioxane, tetrahydrofuran, acetonitrile, toluene, heptane, dichloromethane. The solid form was evaluated and the degree of crystallinity, if it was crystalline, by XRPD. No differences were observed from the succinate salt crystallized form as seen in FIG. 1.

Precipitation by addition of anti-solvent: Addition of anti-solvent—About 30 mg of drug substance was dissolved in a solvent in which solubility was high. To the obtained solutions was added solvents in which drug substance was insoluble or was of low solubility. Precipitates were collected by filtration and investigated as described above. Addition of reversed anti-solvent—About 30 mg of drug substance was dissolved in a solvent in which solubility was high. The obtained solution was added into a solvent which the drug substance was insoluble or was of low solubility. Precipitates were collected by filtration and investigated as described. The precipitation by addition of anti-solvent of the succinate salt was tested in water, methanol, ethanol, THE, and dichloromethane wherein 2-propanol, t-butyl methyl ether, and heptane were used as anti-solvents. The solid form was evaluated and the degree of crystallinity, if it was crystalline, by XRPD. No differences were observed from the succinate salt crystallized form as seen in FIG. 1.

Grinding simulation experiments: About 50 mg of drug substance was ground manually with a mortar and pestle for 3 minutes. The solid form was evaluated and the degree of crystallinity by XRPD. No differences were observed from the succinate salt crystallized form as seen in FIG. 1.

Granulation simulation experiments: To 50 mg of drug substance was added water drop wise until solid was wetted sufficiently. This was then vortexed between each addition and dried under ambient condition for 10 min. The solid form was evaluated and the degree of crystallinity by XRPD. No differences were observed from the succinate salt crystallized form as seen in FIG. 1.

### Example 3—Excipient Compatibility Study

The excipient compatibility study was to test the milled active pharmaceutical ingredient (“API”) (S)-mepazine) with different excipients under designated conditions (40° C./75% R.H. and 60° C.).

Based on the excipient compatibility results of 2 week and 4 week time points, the milled API was relatively stable at both 40° C./75% R.H. and 60° ° C. conditions in ten binary mixtures (Binary 3-5, 7-12 and 14). For binary 1 (Lactose Monohydrate) and 2 (Microcrystalline Cellulose), compared with API, the impurity (RRT, 0.52) referred to (S)-mepazine sulfoxide increased as the stored time increased at both 40° C./75% R.H. and 60° C. conditions. For binary 6 (Crospovidone) and 13 (Polyvinylpyrrolidone, P\TP K29/32), compared with API, the impurity (RRT, 0.52) referred to (S)-mepazine sulfoxide increased as the stored time was increased at 40° C./75% R.H. condition. For binary 15 (Sodium Lauryl Sulfate), the impurity (RRT, 0.52) referred to (S)-mepazine sulfoxide increased as the time and temperature increased at 60° C. condition and the impurity (RRT, 0.56) increased as the stored time increased at 40° C./75% RH. And according to the XRPD result, the form of (S)-mepazine in 15 binaries had no change when compared with raw material. For binary 6 (Crospovidone) and 13 (Polyvinylpyrrolidone, P\TP K29/32) impurity identification (RRT, 0.52), the result of LC-MS indicated the impurity (RRT, 0.52) is the (S)-mepazine sulfoxide.

The HPLC method used for excipient compatibility study is provided in Table 13.

TABLE 13
HPLC Method for Excipient Compatibility Test

| Column | Waters Sunfire C18 column | | |
|---|---|---|---|
| | (3.5 μm, 4.6*150 mm) | | |
| Wavelength | 220 nm | | |
| Column Oven Temp. | 30° C. | | |
| Flow Rate | 1.2 mL/min | | |
| Injection Volume | 5 μL | | |
| Mobile Phases | A: 0.05% TFA in Water (v/V) | | |
| | B: 0.05% TFA in ACN (v/v) | | |
| | Time (min) | A % | B % |
| Gradient Program | 0.00 | 85 | 15 |
| | 7.50 | 70 | 30 |
| | 17.50 | 55 | 45 |
| | 20.00 | 5 | 95 |
| | 25.00 | 5 | 95 |
| | 26.00 | 85 | 15 |
| | 31.00 | 85 | 15 |

| | |
|---|---|
| Run Time | 31 min |
| Needle Wash | MeOH: Water (50:50; v/v) |
| Solvent | |
| Diluent | MeOH: Water (50:50; v/v), |
| | MeOH and EtOH |

Excipient compatibility of (S)-mepazine milled API was investigated with 15 excipients. Details of these binary mixtures are listed in Table 14. Samples were set up at each of these two conditions: 40° C./75% R.H. open, and 60° ° C., closed. Three sampling time points (initial, 2 weeks, and 4 weeks) are specified in Table 15.

The API was set up as a control at each time point and each condition. For the pure excipients, only one bulk sample was set up at each condition. They were analyzed at the initial time point and at other time points if degradation of the active substance was observed. For the 15 binary mixtures, triplicate samples were set up at each time point/condition. Two samples were analyzed for purity and XRPD test, and the third one was kept as a contingency sample. The appearance and XRPD of samples under each condition were recorded at each time point.

TABLE 14
Excipient Compatibility Study Design for Impurities
and Assay Test in Binary Blends

| | | API:Excipient |
|---|---|---|
| No. | Raw Material | Ratio |
| 0 | (S)-Mepazine Succinate Salt | — |
| 1 | Lactose Monohydrate | 1:10 |
| 2 | Microcrystalline Cellulose | 1:10 |
| 3 | Dibasic Calcium Phosphate | 1:10 |
| | dehydrate grade | |
| 4 | Mannitol | 1:10 |
| 5 | Croscarmellose Sodium | 1:1 |
| 6 | Crospovidone | 1:1 |
| 7 | Sodium Starch Glycolate | 1:1 |
| 8 | Hydroxy Propyl Cellulose (EXF) | 1:1 |
| 9 | Magnesium Stearate | 1:1 |
| 10 | Colloidal Silicon Dioxide | 1:1 |
| 11 | Sodium Stearyl Fumarate | 1:1 |
| 12 | Hydroxy Propyl Methyl | 1:10 |
| | Cellulose(HPMC), K15M | |
| 13 | Polyvinylpyrrolidone, PVP | 1:1 |
| | K29/32 | |
| 14 | Polyethylene Oxide | 1:10 |
| 15 | Sodium Lauryl Sulfate | 1:10 |

### Sample Preparation and Storage

Binary mixtures: For each binary mixture, ˜13.8 mg of (S)-mepazine succinate salt milled API (equivalent to the 10 mg active API) and the specified amount of excipient for 15 binary mixtures 1-15 as listed in Table 6 were accurately weighed into a 40 mL clear glass vial and mixed well by Vortex (mixing time ˜15 s). The binary mixture was set up in duplicate.

Excipient blank controls: Each excipient blank was weighed into a 40 mL clear glass vial as excipient blank control. The excipient blank was set up as a single sample.

API control: Approximate: 13.8 mg of API was weighed into a 40 mL clear glass vial. The API control was set up in duplicate.

All the samples for open condition storage were put into an uncapped clear glass vial. The mouth of vials were covered by aluminum foil with pinholes, to avoid cross-contamination, and then stored in the stability chamber. The samples were stored at each condition 40° C./75% R.H. open and 60° C. close conditions and monitored for physical appearance, XRPD, impurities/related substances and assay at each time point, respectively.

### Excipient Compatibility Samples Analysis

All samples at 40° C./75% R.H., and 60° C. for 2 weeks and 4 weeks, were pulled out and cooled to room temperature for physical appearance, total related substances (TRS) and assay analysis. The diluent was accurately added into 40 ml vials and then the solution was sonicated for 10 minutes to facilitate dissolution of the API. Then 1 mL solution was taken out and centrifuged at 14000 rpm for 5 min for twice to separate some undissolved excipients, and then the concentration of supernatants were analyzed by HPLC instrument. The recovery and TRS results of excipient compatibility study at 40° C./75% R.H., and 60° C. are shown in Table 9, and the results of the complete impurity content and XRPD result are listed in Table 7. And the appearance is shown in Table 8.

From the excipient compatibility results of 2 week and 4 week time points, the (S)-mepazine succinate salt milled API was relatively stable at both 40° C./75% R.H. and 60° C. conditions in ten binary mixtures (Binary 3-5, 7-12 and 14). For binary 1 and 2, compared with API, the impurity (RRT, 0.52) increased as the stored time increased at both 40° C./75% R.H. and 60° C. conditions. For binary 6 and 13, compared with API, the impurity (RRT, 0.52) increased as the stored time was increased at 40° C./75% R.H. condition. For binary 15, the impurity (RRT, 0.52) increased as the time and temperature increased at 60° C. condition and the impurity (RRT, 0.56) increased as the stored time increased at 40° C./75% RH. And according to the XRPD result, the form of (S)-mepazine succinate salt in 15 binaries had no change when compared with raw material.

TABLE 15
Results of Excipient Compatibility Study for the Compound stored at initial state, 2 weeks and 4 weeks

| | | | 60° C._close | | | | 40° C./75% RH_open | | | |
|---|---|---|---|---|---|---|---|---|---|---|
| | Initial 0 day | | 2 weeks | | 4 weeks | | 2 weeks | | 4 weeks | |
| | Assay | TRS | Assay | TRS | Assay | TRS | Assay | TRS | Assay | TRS |
| Samples | (%) | (%) | (%) | (%) | (%) | (%) | (%) | (%) | (%) | (%) |
| API control | 100.00 | 0.14 | 99.70 | 0.18 | 102.38 | 0.13 | 100.82 | 0.18 | 100.08 | 0.12 |
| Binary 1 | 100.52 | 0.18 | 99.90 | 0.18 | 100.73 | 0.33* | 100.78 | 0.20 | 100.55 | 0.27* |
| (Lactose | | | | | | | | | | |
| Monohydrate) | | | | | | | | | | |
| Binary 2 | 98.18 | 0.11 | 98.71 | 0.24* | 101.00 | 0.28* | 99.63 | 0.18* | 99.85 | 0.22* |
| (Microcrystalline | | | | | | | | | | |
| Cellulose) | | | | | | | | | | |
| Binary 3 | 99.61 | 0.16 | 99.55 | 0.17 | 101.95 | 0.19 | 101.57 | 0.17 | 99.79 | 0.15 |
| (Dibasic Calcium | | | | | | | | | | |
| Phosphate ) | | | | | | | | | | |
| Binary 4 | 96.75 | 0.16 | 98.13 | 0.17 | 99.88 | 0.16 | 99.06 | 0.15 | 98.87 | 0.13 |
| (Mannitol) | | | | | | | | | | |
| Binary 5 | 98.19 | 0.14 | 100.40 | 0.10 | 102.50 | 0.10 | 98.76 | 0.11 | 100.17 | 0.09 |
| (Croscarmellose | | | | | | | | | | |
| Sodium) | | | | | | | | | | |
| Binary 6 | 99.86 | 0.21 | 100.44 | 0.21 | 103.09 | 0.23 | 99.56 | 0.69* | 101.11 | 0.81* |
| (Crospovidone) | | | | | | | | | | |
| Binary 7 | 97.56 | 0.11 | 98.00 | 0.11 | 101.48 | 0.09 | 97.97 | 0.11 | 99.59 | 0.08 |
| (Sodium Starch Glycolate) | | | | | | | | | | |
| Binary 8 | 95.70 | 0.22 | 98.39 | 0.20 | 99.94 | 0.23 | 93.76 | 0.22 | 98.95 | 0.19 |
| (Hydroxy Propyl | | | | | | | | | | |
| Cellulose) | | | | | | | | | | |
| Binary 9 | 96.93 | 0.19 | 97.74 | 0.19 | 98.99 | 0.17 | 98.58 | 0.18 | 98.23 | 0.14 |
| (Magnesium | | | | | | | | | | |
| Stearate) | | | | | | | | | | |
| Binary 10 | 99.90 | 0.20 | 99.84 | 0.15 | 101.27 | 0.17 | 99.82 | 0.22 | 100.60 | 0.20 |
| (Colloidal Silicon | | | | | | | | | | |
| Dioxide) | | | | | | | | | | |
| Binary 11 | 97.06 | 0.23 | 98.95 | 0.16 | 101.26 | 0.12 | 99.33 | 0.16 | 99.95 | 0.13 |
| (Sodium Stearyl | | | | | | | | | | |
| Fumarate) | | | | | | | | | | |
| Binary 12 (Hydroxy Propyl | 99.17 | 0.16 | 98.61 | 0.16 | 102.11 | 0.12 | 100.81 | 0.18 | 97.21 | 0.17 |
| Methyl Cellulose (HPMC), | | | | | | | | | | |
| K15M) | | | | | | | | | | |
| Binary 13 | 100.00 | 0.40 | 98.49 | 0.38 | 101.77 | 0.42 | 100.09 | 1.17* | 100.44 | 1.14* |
| (PVP K29/32) | | | | | | | | | | |
| Binary 14 | 100.57 | 0.20 | 100.90 | 0.16 | 103.57 | 0.15 | 100.07 | 0.14 | 103.33 | 0.12 |
| (Polyethylene | | | | | | | | | | |
| Oxide) | | | | | | | | | | |
| Binary 15 | 96.97 | 0.19 | 97.06 | 0.17 | 96.83 | 0.27* | 97.88 | 0.25* | 96.81 | 0.28* |
| (Sodium Lauryl | | | | | | | | | | |
| Sulfate) | | | | | | | | | | |

Note:
increased impurity was marked with “*”

TABLE 16
Impurity Content and XRPD Results for initial state, 2 weeks, and
4 weeks at 60° C. and 40° C./75% RH condition.

| | | | | Area (100%) | | | |
|---|---|---|---|---|---|---|---|
| Samples | RRT (relative retention time) | | | 0.52 | 0.56 | 1.00 | XRPD |
| API control | Initial | 0 | | 0.04 | 0.10 | 99.86 | — |
| | 60° C. close | 2 | weeks | 0.03 | 0.16 | 99.82 | No change |
| | | 4 | weeks | 0.04 | 0.09 | 99.87 | No change |
| | 40° C./75% RH open | 2 | weeks | 0.03 | 0.15 | 99.82 | No change |
| | | 4 | weeks | 0.02 | 0.10 | 99.88 | No change |
| Binary 1 | Initial | 0 | day | 0.08 | 0.10 | 99.82 | No change |
| (Lactose Monohydrate) | 60° C. close | 2 | weeks | 0.07 | 0.11 | 99.82 | No change |
| | | 4 | weeks | 0.25* | 0.08 | 99.67 | No change |
| | 40° C./75% RH open | 2 | weeks | 0.08 | 0.12 | 99.80 | No change |
| | | 4 | weeks | 0.17* | 0.09 | 99.73 | No change |
| Binary 2 | Initial | 0 | day | 0.03 | 0.08 | 99.89 | No change |
| (Microcrystalline | 60° C. close | 2 | weeks | 0.14* | 0.10 | 99.76 | No change |
| Cellulose) | | 4 | weeks | 0.19* | 0.08 | 99.73 | No change |
| | 40° C./75% RH open | 2 | weeks | 0.07* | 0.11 | 99.82 | No change |
| | | 4 | weeks | 0.12* | 0.10 | 99.78 | No change |
| Binary 3 | Initial | 0 | day | 0.07 | 0.09 | 99.84 | No change |
| (Dibasic Calcium | 60° C. close | 2 | weeks | 0.06 | 0.11 | 99.83 | No change |
| Phosphate) | | 4 | weeks | 0.09 | 0.09 | 99.82 | No change |
| | 40° C./75% RH open | 2 | weeks | 0.06 | 0.10 | 99.83 | No change |
| | | 4 | weeks | 0.06 | 0.09 | 99.85 | No change |
| Binary 4 | Initial | 0 | day | 0.05 | 0.11 | 99.84 | No change |
| (Mannitol) | 60° C. close | 2 | weeks | 0.05 | 0.12 | 99.83 | No change |
| | | 4 | weeks | 0.04 | 0.12 | 99.85 | No change |
| | 40° C./75% RH open | 2 | weeks | 0.03 | 0.12 | 99.85 | No change |
| | | 4 | weeks | 0.02 | 0.11 | 99.87 | No change |
| Binary 5 | Initial | 0 | day | 0.07 | 0.07 | 99.86 | No change |
| (Croscarmellose Sodium) | 60° C. close | 2 | weeks | 0.03 | 0.07 | 99.90 | No change |
| | | 4 | weeks | 0.04 | 0.06 | 99.90 | No change |
| | 40° C./75% RH open | 2 | weeks | 0.03 | 0.09 | 99.89 | No change |
| | | 4 | weeks | 0.03 | 0.07 | 99.91 | No change |
| Binary 6 | Initial | 0 | day | 0.10 | 0.10 | 99.79 | No change |
| (Crospovidone) | 60° C. close | 2 | weeks | 0.10 | 0.11 | 99.79 | No change |
| | | 4 | weeks | 0.13 | 0.10 | 99.77 | No change |
| | 40° C./75% RH open | 2 | weeks | 0.58* | 0.11 | 99.31 | No change |
| | | 4 | weeks | 0.71* | 0.11 | 99.19 | No change |
| Binary 7 | Initial | 0 | day | 0.04 | 0.06 | 99.89 | No change |
| (Sodium Starch Glycolate) | 60° C. close | 2 | weeks | 0.04 | 0.07 | 99.89 | No change |
| | | 4 | weeks | 0.02 | 0.07 | 99.91 | No change |
| | 40° C./75% RH open | 2 | weeks | 0.04 | 0.07 | 99.90 | No change |
| | | 4 | weeks | 0.02 | 0.06 | 99.92 | No change |
| Binary 8 | Initial | 0 | day | 0.13 | 0.10 | 99.78 | No change |
| (Hydroxy Propyl Cellulose) | 60° C. close | 2 | weeks | 0.09 | 0.11 | 99.80 | No change |
| | | 4 | weeks | 0.12 | 0.11 | 99.77 | No change |
| | 40° C./75% RH open | 2 | weeks | 0.11 | 0.11 | 99.78 | No change |
| | | 4 | weeks | 0.09 | 0.10 | 99.81 | No change |
| Binary 9 | Initial | 0 | day | 0.07 | 0.12 | 99.81 | No change |
| (Magnesium Stearate) | 60° C. close | 2 | weeks | 0.05 | 0.14 | 99.81 | No change |
| | | 4 | weeks | 0.04 | 0.13 | 99.83 | No change |
| | 40° C./75% RH open | 2 | weeks | 0.06 | 0.12 | 99.82 | No change |
| | | 4 | weeks | 0.03 | 0.12 | 99.86 | No change |
| Binary 10 | Initial | 0 | day | 0.11 | 0.09 | 99.80 | No change |
| (Colloidal Silicon Dioxide) | 60° C. close | 2 | weeks | 0.04 | 0.11 | 99.85 | No change |
| | | 4 | weeks | 0.07 | 0.10 | 99.83 | No change |
| | 40° C./75% RH open | 2 | weeks | 0.10 | 0.12 | 99.78 | No change |
| | | 4 | weeks | 0.12 | 0.08 | 99.80 | No change |
| Binary 11 | Initial | 0 | day | 0.14 | 0.09 | 99.77 | No change |
| (Sodium Stearyl Fumarate) | 60° C. close | 2 | weeks | 0.05 | 0.11 | 99.84 | No change |
| | | 4 | weeks | 0.03 | 0.10 | 99.88 | No change |
| | 40° C./75% RH open | 2 | weeks | 0.04 | 0.12 | 99.84 | No change |
| | | 4 | weeks | 0.02 | 0.11 | 99.87 | No change |
| Binary 12 | Initial | 0 | day | 0.04 | 0.12 | 99.84 | No change |
| (Hydroxy Propyl Methyl | 60° C. close | 2 | weeks | 0.05 | 0.11 | 99.84 | No change |
| Cellulose, K15M) | | 4 | weeks | 0.04 | 0.08 | 99.88 | No change |
| | 40° C./75% RH open | 2 | weeks | 0.07 | 0.12 | 99.82 | No change |
| | | 4 | weeks | 0.11 | 0.06 | 99.83 | No change |
| Binary 13 | Initial | 0 | day | 0.30 | 0.11 | 99.60 | No change |
| (Polyvinylpyrrolidone, PVP | 60° C. close | 2 | weeks | 0.27 | 0.11 | 99.62 | No change |
| K29/32) | | 4 | weeks | 0.33 | 0.09 | 99.58 | No change |
| | 40° C./75% RH open | 2 | weeks | 1.05* | 0.12 | 98.83 | No change |
| | | 4 | weeks | 1.03* | 0.11 | 98.87 | No change |
| Binary 14 | Initial | 0 | day | 0.14 | 0.06 | 99.80 | No change |
| (Polyethylene Oxide) | 60° C. close | 2 | weeks | 0.09 | 0.07 | 99.84 | No change |
| | | 4 | weeks | 0.09 | 0.06 | 99.85 | No change |
| | 40° C./75% RH open | 2 | weeks | 0.09 | 0.06 | 99.86 | No change |
| | | 4 | weeks | 0.06 | 0.06 | 99.88 | No change |
| Binary 15 | Initial | 0 | day | 0.16 | 0.04 | 99.81 | No change |
| (Sodium Lauryl Sulfate) | 60° C. close | 2 | weeks | 0.09 | 0.08 | 99.83 | No change |
| | | 4 | weeks | 0.21* | 0.06 | 99.73 | No change |
| | 40° C./75% RH open | 2 | weeks | 0.11 | 0.15* | 99.75 | No change |
| | | 4 | weeks | 0.10 | 0.17* | 99.73 | No change |

Note:
Only impurities higher than 0.05% was reported and increased impurity was marked with “*”.

Various formulations were prepared according to the disclosure herein. The API of the formulations below is (S)-mepazine succinate salt form. The formulations were prepared as follows:

Modified release (“MR”) formulations—MR1, MR2, MR3, and MR4 are shown in Table 17.

TABLE 17

| | Formulation details | | | |
|---|---|---|---|---|
| | MR1 | MR2 | MR3 | MR4 |
| Ingredient Name | % w/w | | | |
| API | 25.00 | 25.00 | 25.00 | 25.00 |
| Microcrystalline Cellulose | 15.00 | 15.50 | 14.00 | 15.00 |
| Lactose Monohydrate | 41.00 | 45.50 | 42.00 | 44.75 |
| Hydroxypropyl | 15.00 | 10.00 | N/A | N/A |
| Methylcellulose K4M* | | | | |
| Hydroxypropyl | N/A | N/A | 15.00 | 10.00 |
| Methylcellulose K100 LV* | | | | |
| Hydroxypropylcellulose | 3.00 | 3.00 | 3.00 | 7.50 |
| Silicon dioxide | 0.50 | 0.50 | 0.50 | 1.00 |
| Magnesium Stearate | 0.50 | 0.50 | 0.50 | 1.25 |
| Total | 100.00 | 100.00 | 100.00 | 100.00 |

*K4M is hydroxypropyl methylcellulose having a viscosity of 4,000 cP at 2% in water
K100LV is hydroxypropyl methylcellulose having a viscosity of 100 cP at 2% in water

Immediate release formulations—IR1, IR2, and IR3 are shown in Table 18.

TABLE 18

| | Formulation details | | |
|---|---|---|---|
| | IR1 | IR2 | IR3 |
| Ingredient Name | % w/w | | |
| API | 25.00 | 25.00 | 25.00 |
| Microcrystalline Cellulose | 17.00 | 17.00 | 16.00 |
| Lactose Monohydrate | 51.00 | 50.00 | 50.00 |
| Hydroxypropylcellulose | 3.00 | 5.00 | 5.00 |
| Sodium Carboxymethyl Cellulose | 3.00 | 2.00 | N/A |
| Crospovidone (polyvinylpyrrolidone) | N/A | N/A | 3.00 |
| Silicon dioxide | 0.50 | 0.50 | 0.50 |
| Magnesium Stearate | 0.50 | 0.50 | 0.50 |
| Total | 100.00 | 100.00 | 100.00 |

Immediate release formulations IR4, IR5 and IR6 are shown in Table 19.

TABLE 19

| | Formulation details | | |
|---|---|---|---|
| | IR4 | IR5 | IR6 |
| Ingredient Name | % w/w | | |
| API | 25.00 | 25.00 | 25.00 |
| Microcrystalline Cellulose | 12.00 | 15.00 | 17.00 |
| Lactose Monohydrate | 34.00 | 46.00 | 50.00 |
| Sodium Lauryl Sulfate | 20.00 | 5.00 | N/A |
| Hydroxypropylcellulose | 5.00 | 5.00 | 3.00 |
| Sodium Carboxymethyl Cellulose | 3.00 | 3.00 | 3.00 |
| Silicon dioxide | 0.50 | 0.50 | 1.00 |
| Magnesium Stearate | 0.50 | 0.50 | 1.00 |
| Total | 100.00 | 100.00 | 100.00 |

### Dissolution Results for Tablets

Preparation procedures for dissolution medium:

To make 10 L of pH 1.2 HCl solution, for example, add 85 ml of hydrochloric acid into a suitable container, dilute to volume 10 L with purified water and mix well. Verify the pH value is pH 1.2±0.05 or not If not, adjust pH to 1.2±0.05 with purified water or hydrochloric acid.

To make 10 L of pH 6.8 phosphate buffer solution, for example, weigh 68.05 g of KH₂PO₄ and 8.95 of NaOH into a suitable container, then dissolve and dilute purified water to volume 10 L. Adjust to pH 6.8±0.05 with 50% (w/v) NaOH in purified water and mix well.

TABLE 20
Dissolution parameters

| | |
|---|---|
| Apparatus | USP Apparatus 2 (Paddles) |
| Bath Type | Water bath |
| Rotation Speed | 50 rpm |
| Dissolution Medium | pH 1.2 HCl buffer |
| | pH 6.8 Phosphate buffer |
| Medium Volume | 900 mL |
| Medium Temperature | 37.0 ± 0.5° C. |
| Sampling Volume | Sampling volume: 1.5 mL |
| | Waste drop volume: 0.5 mL |
| | Prime volume: 7 mL |
| Sampling Time | 5, 10, 15, 20, 30, 45, 60 and 75 |
| | minutes |
| Clarification | 10 μm full flow filter |

The results of the dissolution testing for IR Tablets are in Table 21 and shown in FIG. 27.

TABLE 21

| | Dissolu- | | | | | | | | |
|---|---|---|---|---|---|---|---|---|---|
| | tion | Time Point (min)/% Release | | | | | | | |
| | method | 5 | 10 | 15 | 20 | 30 | 45 | 60 | 75 |
| IR1 | pH 1.2 | 91 | 102 | 102 | 102 | 102 | 102 | 103 | 103 |
| IR2 | HCl | 13 | 32 | 67 | 93 | 102 | 102 | 102 | 102 |
| IR3 | solution | 14 | 36 | 68 | 96 | 102 | 103 | 103 | 103 |
| IR4 | | 2 | 4 | 5 | 6 | 8 | 10 | 12 | 15 |
| IR5 | | 9 | 27 | 48 | 61 | 64 | 70 | 74 | 76 |
| IR6 | | 88 | 101 | 102 | 102 | 102 | 102 | 102 | 102 |

The dissolution testing of the IR tablet formulations unexpectedly showed that formulations including sodium lauryl sulfate, IR4 and IR5, had decreased dissolution in pH 1.2 HCl solutions. In general, an ordinary skilled artisan would expect pharmaceutical formulations including SLS to show increased dissolution in acidic and basic solutions, however, that was unexpectedly not the result disclosed herein. FIG. 27 shows IR4 having less than 20% dissolution over a period of time of 75 minutes and IR5 having less than 80% dissolution of the formulation. In contrast, the IR formulations, IR1, IR2, IR3, IR6, all have 80% dissolution or more after a period of 20 minutes and 80% dissolution or more after a period of 30 minutes.

Comparative dissolution profile of tablets 50 mg, immediate release tablets in pH 6.8 Phosphate buffer. Results of the comparison in Table 22.

TABLE 22

| | Dissolution Medium | | | | | | | | |
|---|---|---|---|---|---|---|---|---|---|
| | pH 6.8 Phosphate buffer | | | | | | | | |
| | Test conditions | | | | | | | | |
| | Apparatus: | | RPM: | | | Volume: | | | |
| | USP-II | | 50 RPM | | | 900 mL | | | |
| | API % Release (50 mg) | | | | | | | | |
| | Time in | | | | | | | | |
| | minutes | 5 | 10 | 15 | 20 | 30 | 45 | 60 | 75 |
| IR1 | Mean | 85 | 99 | 100 | 101 | 101 | 101 | 101 | 101 |
| IR2 | Mean | 69 | 97 | 100 | 102 | 103 | 103 | 103 | 103 |
| IR3 | Mean | 60 | 96 | 101 | 101 | 101 | 101 | 101 | 101 |

The results of the dissolution testing for MR Tablets are in Table 23.

TABLE 23

| | | Time Point (min)/% Release | | | | | | | | | |
|---|---|---|---|---|---|---|---|---|---|---|---|
| | | | | | | 150 | 180 | 240 | 360 | 480 | 720 |
| | Dissolution | 30 | 60 | 90 | 120 | Buffer stage (Including acid stage sampling time | | | | | |
| | method | Acid stage-pH 1.2 | | | | point) | | | | | |
| MR1 | Option B in | 16 | 24 | 30 | 35 | 40 | 45 | 50 | 58 | 64 | 73 |
| MR2 | USP 711 | 18 | 29 | 37 | 44 | 50 | 57 | 66 | 83 | 94 | 99 |
| MR3 | | 19 | 33 | 47 | 59 | 74 | 82 | 91 | 99 | 99 | 99 |
| MR4 | | 29 | 51 | 72 | 87 | 99 | 104 | 104 | 104 | 104 | 104 |

The modified release tablets showed differing dissolution profiles in the two-stage dissolution process. Modified release tablets with higher amounts of polymer tended to dissolve slower. Modified release tablets with higher molecular weight polymers tended to dissolve slower.

Dissolution testing summary for IR1 of at least 4 weeks is shown in Table 24 below.

TABLE 24

| | Dissolution | Time Point (min)/% Release | | | | | | | |
|---|---|---|---|---|---|---|---|---|---|
| | method | 5 | 10 | 15 | 20 | 30 | 45 | 60 | 75 |
| IR1 at Initial | pH 1.2 HCl | 91 | 102 | 102 | 102 | 102 | 102 | 103 | 103 |
| IR1- at 17 days | solution | 89 | 104 | 104 | 104 | 104 | 104 | 104 | 104 |
| under 40° C. ± 2° | | | | | | | | | |
| C./75% ± 5% RH | | | | | | | | | |
| IR1- at 4 weeks | | 94 | 103 | 103 | 103 | 101 | 102 | 102 | 102 |
| under 40° C. ± 2° | | | | | | | | | |
| C./75% ± 5% RH | | | | | | | | | |

| | | | | | | | | |
|---|---|---|---|---|---|---|---|---|
| RSD | 2.8% | 1.0% | 1.0% | 1.0% | 1.5% | 1.1% | 1.0% | 1.0% |

Dissolution testing summary for IR2 of at least 4 weeks is shown in Table 25 below.

TABLE 25

| | Dissolution | Time Point (min)/% Release | | | | | | | |
|---|---|---|---|---|---|---|---|---|---|
| Formulation | method | 5 | 10 | 15 | 20 | 30 | 45 | 60 | 75 |
| IR2 at Initial | pH 1.2 HCl | 13 | 32 | 67 | 93 | 102 | 102 | 102 | 102 |
| IR2- at 17 days under 40° | solution | 14 | 56 | 92 | 102 | 104 | 104 | 104 | 104 |
| C. ± 2° C./75% ± 5% RH | | | | | | | | | |
| IR2- at 4 weeks under 40° | | 13 | 54 | 91 | 100 | 104 | 105 | 105 | 105 |
| C. ± 2° C./75% ± 5% RH | | | | | | | | | |

| | | | | | | | | |
|---|---|---|---|---|---|---|---|---|
| RSD | 4.3% | 28.1% | 17.0% | 4.8% | 1.1% | 1.5% | 1.5% | 1.5% |

As shown in the Table 24 and Table 25, both IR1 and IR2 high dissolution (e.g., 80% or higher) at the 20 minute time point or better after 17 days and 4 weeks under conditions of 40° C.±2° C./75%±5% RH.

### Example 4—CYP Phenotyping of (S)-Mepazine Salts

Two test systems were used to determine which CYP enzyme is responsible for the metabolism of (S)-mepazine:

Recombinant CYP enzymes: (S)-mepazine was incubated with heterologously expressed individual human CYP1A2, CYP2A6, CYP2B6, CYP2C₈, CYP2C9, CYP2C19, CYP2D6, CYP2E1, CYP3A4 and CYP3A5 isoforms. These incubations were carried out at a concentration of 1 UM and contained 50 pmol/ml of CYP protein. Samples were taken at 0, 5, 10, 15, 20 and 30 minutes. The CLᵢₙₜ and t_{1/2} were calculated from the data.

Human liver microsomes with or without specific inhibitors: By using human liver microsomes with chemical inhibitor, compared to without chemical inhibitor, the incubation was carried out at a concentration of 1 UM with samples taken 0, 15, 30, 45 and 60 min, and analyzed by UPLC-MS/MS to quantitate the concentration of parent remaining. The % remaining was calculated.

Intrinsic clearance (CLᵢₙₜ) and the half-life (t½) of (S)-mepazine are summarized in Table 26. The % remaining of (S)-mepazine in human liver microsomes in the presence and absence of chemical inhibitors for specific CYP enzymes are summarized in Table 27.

TABLE 26
Intrinsic clearance (CLᵢₙₜ) and the half-life of (S)-mepazine
per CYP (mean ± SD, n = 3)

| | CLᵢₙₜ | t_{1/2} | Contribution |
|---|---|---|---|
| CYP Isoform | (μL/min/pmol CYP) | (min) | % |
| CYP1A2 | 0.272 ± 0.0339 | 51.5 ± 5.99 | 9.61 |
| CYP2A6 a | 0.00 | ∞ | 0.00 |
| CYP2B6 a | 0.00 | ∞ | 0.00 |
| CYP2C8 | 0.254 ± 0.0720 | 57.2 ± 14.4 | 12.8 |
| CYP2C9 b | 0.109 | 127 | 8.24 |
| CYP2C19 | 0.537 ± 0.0361 | 25.9 ± 1.68 | 8.01 |
| CYP2D6 | 1.40 ± 0.112 | 9.94 ± 0.819 | 11.0 |
| CYP2E1 a | 0.00 | ∞ | 0.00 |
| CYP3A4 | 0.593 ± 0.0562 | 23.5 ± 2.35 | 50.3 |
| CYP3A5 b | 0.0829 | 167 | 0.0651 |

a If turnover was not significant (t-test with p < 0.05 was not obtained) and the percentage remaining at the last time point was > 80%, t1/2 and CLint were reported as “∞” and “0.00”, respectively.
b Turnover was observed for one or two replicates of the incubations, the t1/2 and the Clint was not calculated for other replicates (turnover was not significant (t-test) and the percentage remaining at the last time point was ≥80%). The t1/2 was reported as minimum and Clint was reported as maximum.

TABLE 27
% Remaining of (S)-mepazine in human liver microsomes in the presence
and absence of chemical inhibitors (mean ± SD, n = 3).

| | % Remaining at 60 | |
|---|---|---|
| CYPs (Inhibitor) | min (%) | Inhibition % |
| Without inhibitor | 44.7 ± 2.57 | 0.00 |
| CYP1A2 (Furafylline) | 55.3 ± 4.07 | 31.6 |
| CYP2A6 (Tranylcypromine) | 53.4 ± 2.69 | 25.6 |
| CYP2B6 (Ticlopidine) | 52.6 ± 3.79 | 22.8 |
| CYP2C8 (Montelukast) | 50.5 ± 4.24 | 19.3 |
| CYP2C9 (Sulfaphenazole) | 57.4± 2.68 | 33.9 |
| CYP2C19 (N-3-benzylnirvanol) | 53.1 ± 1.75 | 24.3 |
| CYP2D6 (Quinidine) | 80.9 ± 3.69 | 74.4 |
| CYP3A (Ketoconazole) | 50.2 ± 4.00 | 21.4 |

In recombinant CYP Isoform incubation: The CYP2A6, CYP2B6 and CYP2E1 were not found to substantially metabolize (S)-mepazine in this system. The metabolism of (S)-mepazine in CYP1A2, CYP2C8, CYP2C19, CYP2D6 and CYP3A4 was observed, and the CLᵢₙₜ value was 0.272 μL/min/pmol, 0.254 μL/min/pmol, 0.537 μL/min/pmol, 1.40 μL/min/pmol, 0.593 μL/min/pmol, respectively. In CYP2C9 and CYP3A5 incubations, (S)-mepazine turnover was observed in one of the three replicates, and the CLᵢₙₜ value were 0.109 μL/min/pmol and 0.0829 μL/min/pmol, respectively. The contribution % of CYP1A2, CYP2C₈, CYP2C₉, CYP2C19, CYP2D6, CYP3A4 and CYP3A5 in (S)-mepazine was 9.61, 12.8, 8.24, 8.01, 11.0, 50.3 and 0.0651, respectively.

In human liver microsome incubations with and without specific inhibitors, the metabolism of (S)-mepazine was inhibited significantly when the specific inhibitor of CYP1A2, CYP2A6, CYP2B6, CYP2C9, CYP2C19, CYP2D6 and CYP3A was added, and the inhibition % were 31.6%, 25.6%, 22.8%, 33.9%, 24.3%, 74.4% and 21.4%, respectively. The inhibition of (S)-mepazine in human liver microsomes with inhibitor of CYP2C8 was <20%, so CYP2C8 may have less contribution for the metabolism of (S)-mepazine.

Based on the data obtained using both expressed human cytochrome P450s and liver microsomes with specific inhibitors, the results from the two test systems were relatively consistent. CYP2D6 is major CYP isoform participating in (S)-mepazine metabolism.

### Example 5—Dog PK Screening

Plasma pharmacokinetics (PK) of (S)-mepazine from different tablet formulations in beagle dogs: Tablets formulations IR1, IR2, MR1 and MR2 are disclosed in the above examples and results are shown in Table 25. table

Multi-phase PK studies were conducted to evaluate (S)-mepazine tablet formulations in the same group of non-naive male beagle dogs (n=5). Two immediate release (IR) formulations (IR1 and IR2) and two modified release (MR) formulations (MR1 and MR2) were tested. Each tablet contained 50 mg of (S)-mepazine free base. In the study design, a group of male beagle dogs was fed with standard canned dog food (half a can of enriched canned food) 1 hour before dosing and pre-treated with pentagastrin (at 6 μg/kg or 0.024 mL/kg by intramuscular injection of 0.25 mg/mL solution) 30 minutes before tablet dosing. Each tablet formulation (containing 50 mg of (S)-mepazine free base) was administered orally representing about 5 mg/kg/dose based on dog weight followed by approximately 40 mL of drinking water. The washout period between each phase was at least 7 days. Plasma samples were collected at pre-dose (0), 0.083, 0.25, 0.5, 0.75, 1, 2, 4, 6, 8, 12 and 24 hours post-dose. Concentrations of (S)-mepazine in plasma samples were determined by an LC-MS/MS method. The results are summarized in Table 25.

TABLE 25
Mean Plasma Pharmacokinetic Parameters of
(S)-Mepazine in Four Tablet Formulations

| | Tablet IR1 | Tablet IR2 | Tablet MR1 | Tablet MR2 |
|---|---|---|---|---|
| | 1 × 50 mg, | 1 × 50 mg, | 1 × 50 mg, | 1 × 50 mg, |
| | 5 mg/kg/dose, PO | 5 mg/kg/dose, PO | 5 mg/kg/dose, PO | 5 mg/kg/dose, PO |
| Parameter | (n = 5) | (n = 5) | (n = 5) | (n = 5) |
| Tₘₐₓ (h) | 1.5 ± 0.69 | 1.6 ± 0.55 | 2.8 ± 1.1 | 2.6 ± 1.34 |
| Cₘₐₓ (ng/ml) | 130 ± 38.6 | 128 ± 22.0 | 91.5 ± 26.4 | 121 ± 21.1 |
| t_{1/2} (h) | 5.37 ± 0.36 | 5.34 ± 0.33 | 5.14 ± 0.48 | 5.37 ± 0.86 |
| AUC_{0-inf} | 1026 ± 300 | 976 ± 246 | 740 ± 194 | 996 ± 237 |
| (ng · h/mL) | | | | |

AUC = area under the curve; Cₘₐₓ = peak (or maximum) concentration; IR = immediate release; MR = modified release; PO = oral; t_{1/2} = half-life; Tₘₐₓ = time to reach maximum concentration.

The modified release tablet formulations (MR1 and MR2) showed a delay in reaching Tmax. All tablet formulations exhibited similar Cₘₐₓ and AUC.

### Example 6—Synthesis of (S)-Mepazine and (S)-Mepazine Succinate Salt

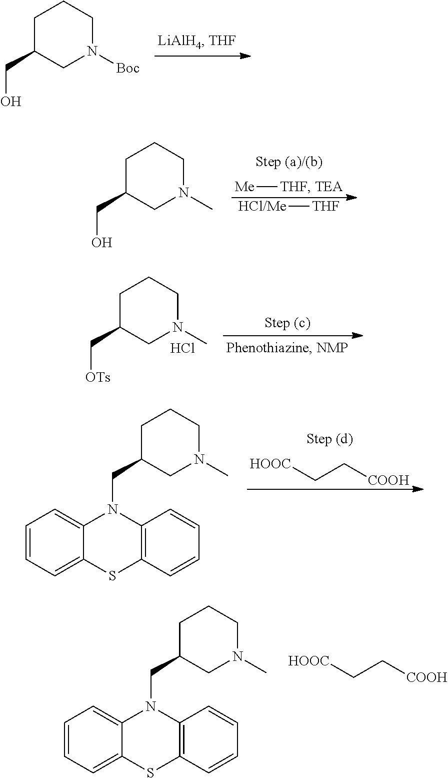

The synthesis of (S)-mepazine and (S)-mepazine succinate salt disclosed herein differs from the previously reported synthesis of (S)-mepazine and (S)-mepazine HCl salt in a variety of ways including, but not limited to, step 1 included the use a water/THE solution to quench the reaction, the use of NaOH/MgSO₄ instead of potassium sodium tartrate solution, and the preparation of a 2-Me-THE solution to carry over to the next step; step 2 included a different reaction solvent, e.g., 2-Me-THF, and the workup further included making an HCl salt and slurry to purge impurities; step 3 included a different reaction solvent, e.g., NMP, and the workup further included making the HCl salt and followed by neutralization of the HCl salt for purification; and, step 4 included the formation of the (S)-mepazine succinate salt.

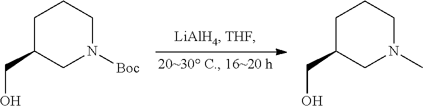

(S)-(1-methylpiperidin-3-yl)methanol (Compound J): To a first vessel was charged 99.0 g of tert-butyl (3S)-3-(hydroxymethyl)piperidine-1-carboxylate and 1200 ml of THF. The solution was adjusted to 20-30° C. LiAlH₄ was charged to the first vessel and the mixture was stirred for 16 hours at 20-30° C. The first vessel was cooled to −5-5° C. and a mixture of 10 volumes (mL/g of tert-butyl (3S)-3-(hydroxymethyl)piperidine-1-carboxylate) of THF and 45 mL of water was charged into the first vessel over 3 hours. A mixture of 13.5 g of NaOH and 31.5 mL of water were charged into the first vessel and a further 135 ml of water was charged to the first vessel. 100 g of MgSO₄ was added to the first vessel and then the mixture was warmed to 20-30° C. for 1.5 hours. The resulting mixture was filtered and 8 volumes of 2-MeTHF was added and kept at 20-30° C. for 16 hours. The resulting mixture was filtered and washed with 1 volume of 2-MeTHF. The product was washed again with 2-Me-THF and concentrated to 5 volumes. The product was obtained in an amount of 523.3 g and 88.3% purity with a 79.8% yield.

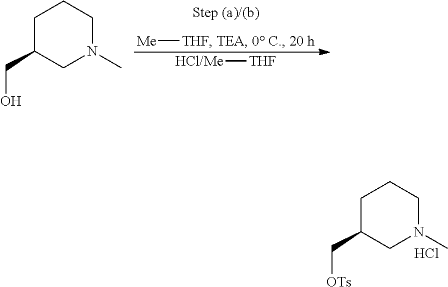

[(3S)-1-methyl-3-piperidyl]methyl 4-methylbenzenesulfonate hydrochloride salt (Compound K hydrochloride salt, wherein LG is OTs): To a vessel was charged a 1230.0 g solution of (S)-(1-methylpiperidin-3-yl)methanol in THF (10.4% (S)-(1-methylpiperidin-3-yl)methanol). 600 mL of 2-MeTHF was charged to the vessel and the solution was cooled to −5-5° C. Et₃N (450.0 g, 4.492 eq.) was charged to the vessel slowly. Tosyl chloride (TsCl, 397.0 g, 2.103 eq.) was charged to the solution at −5-5° C. and the solution was brought back to 20-30° C., and further stirred for 20 hours. The product [(3S)-1-methyl-3-piperidyl]methyl 4-methylbenzenesulfonate was synthesized in 99.72% purity.

600 ml of 2-MeTHF was charged to the vessel with the [(3S)-1-methyl-3-piperidyl]methyl 4-methylbenzenesulfonate and the temperature was adjusted to 0-10° C. The solution was washed with 800 ml of saturated NaHCO₃ three times. The solution was further washed with 800 ml of water and washed with 800 mL of brine. The organic layer was then let stand for 60 hours at 0-10° C., was concentrated to 8 volumes (mL/g of [(3S)-1-methyl-3-piperidyl]methyl 4-methylbenzenesulfonate) in 2-MeTHF, and let stand for 16 hours. 770.5 g 15% (wt %) HCl/2-MeTHE solution was added dropwise to the solution and the temperature was kept at 10-20° C. and stirred for 2-3 hours. The solution was then filtered under an N2 atmosphere and washed with 2-MeTHF twice (120 mL each). The solution was charged with 2-MeTHF (1600 mL) and acetonitrile (800 mL) and was stirred for 16 hours at 20-30° C. The solution was then filtered and washed two times with 120 mL of 2-MeTHF and dried at 25-30° ° C. for 16 hours. The product [(3S)-1-methyl-3-piperidyl]methyl 4-methylbenzenesulfonate hydrochloride salt was recovered as a solid in a 80.46% yield, purity of 98.35%.

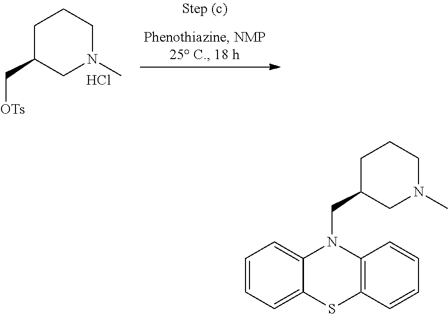 [SMILES: Cc1ccc(S(=O)(=O)OC[C@H]2CCCN(C)C2)cc1 ; Cl ; CN1CCC[C@H](CN2c3ccccc3Sc3ccccc32)C1]

(S)-Mepazine: 1380 mL of N-methyl-2-pyrrolidone (NMP) was charged to a first vessel and the temperature was adjusted to 0-10° C. Sodium hydride (86.5 g, 3.007 eq.) was added to the first vessel and followed by the slow addition of 10H-phenothiazine (158.0 g, 1.103 eq.). The mixture was stirred for 0.5 hours at 0-10° C. and then slowly charged with 230 g of [(3S)-1-methyl-3-piperidyl]methyl 4-methylbenzenesulfonate hydrochloride salt. The solution was warmed to 20-30° C. and stirred for 20 hours. The solution was let stand for 24 hours and charged with 2300 mL of methyl tert-butyl ether (MTBE). A second vessel was charged with NH₄Cl (193.0 g, 5.017 eq.) and 2300 ml of water. The contents of the first vessel were slowly added to the second vessel at 15-25° C. and stirred for 1 hour. The water layer was separated out and further washed with 1150 mL of MTBE. The organic layers were combined and washed twice with 1150 ml of water each wash. The aqueous layer is further washed with 460 ml of water and charged with 35% HCl (150 g) at 20-30° C. The aqueous layer and organic layer were then combined and the stirred for 1 hour at 0-10° C. The aqueous layer and organic layer were then separated and the aqueous layer was washed with 2300 ml of MTBE and 1150 ml of MTBE. The organic layer is again washed with water and Na₂CO₃ (230 g) is added and stirred for 0.5 hours. The organic layer is separated and washed twice more with water. The organic layer is concentrated to 6 v. (S)-mepazine was obtained in 1206 g; purity: 99.3% and yield: 89.675%.

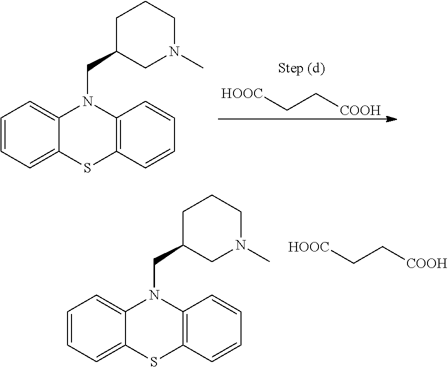 [SMILES: CN1CCC[C@H](CN2c3ccccc3Sc3ccccc32)C1 ; O=C(O)CCC(=O)O ; CN1CCC[C@H](CN2c3ccccc3Sc3ccccc32)C1 ; O=C(O)CCC(=O)O]

(S)-mepazine succinate salt: 199.2 g of (S)-mepazine in a 1200 g total solution of acetone was charged into a first vessel and distilled to 3-5 volumes below 50° C. The solution was concentrated to 2 mL and the first vessel was charged with 2300 mL (10 vol) of acetone. The distillation and addition of acetone were repeated four times, and after, the first vessel was left to stand for 16 hours. A second vessel was charged with succinic acid (79.6 g, 1.051 eq.) and 3000 ml of acetone, and let stir for 1 hour at 20-30° C. A portion of the succinic acid solution (250 g) in the second vessel was charged to the first vessel. A 1.1 g of seed crystals were charged into the first vessel and stirred for 4 hours at 22-27° C. The remaining solution in the second vessel was added dropwise to the first vessel over 14 hours. The first vessel was stirred for 6 hours at 22-27° C. and cooled to 0-5° C. in 3-6 hours. The first vessel was let stir at that temperature for 60 hours. The contents of the first vessel were then filtered and washed with pre-cooled acetone (250 mL) twice. The crystals were dried under vacuum at 25-30° ° C. for 24 hours. 240.1 g of (S)-mepazine succinate salt was obtained in 88.5% yield, a chiral purity of 100.0%, and purity of 99.82%.

What is claimed:

1. A salt having a structure: 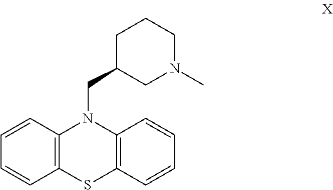 [SMILES: CN1CCC[C@H](CN2c3ccccc3Sc3ccccc32)C1]
wherein X comprises a conjugate base of an organic diacid.

2. The salt of claim 1, wherein X is succinate, fumarate, hemi-fumarate, tartrate, malate, glutamate, or adipate.

3. The salt of claim 1 or 2, wherein X is succinate.

4. The salt of any one of claims 1 to 3, in a crystalline form.

5. The salt of claim 4, wherein X is succinate and the crystalline form is characterized by an X-ray powder diffraction (XRPD) pattern comprising peaks at 12.0, 16.8, and 18.6±0.2° 2θ using Cu Kα radiation.

6. The salt of claim 5, further characterized by XRPD pattern peaks at 10.8, 16.0, 17.6, 19.3, and 23.2±0.2° 2θ using Cu Kα radiation.

7. The salt of claim 5, further characterized by XRPD pattern peaks at 3.4, 4.1, 13.5, 14.1, 20.0, 21.4, 21.7, 25.5, 27.0, 27.5, and 30.9±0.2° 2θ using Cu Kα radiation.

8. The salt of any one of claims 5 to 7, having an XRPD pattern substantially as shown in FIG. 1.

9. The salt of any one of claims 5 to 8, having an endothermic transition at 156° ° C. to 176° C., as measured by differential scanning calorimetry.

10. The salt of claim 9, wherein the endothermic transition is at 166° C.±3° C.

11. The salt of any one of claims 5 to 10, having a thermogravimetric analysis (“TGA”) substantially as shown in FIG. 2.

12. The salt of claim 1 or 2, wherein X is fumarate.

13. The salt of claim 12, in a crystalline form.

14. The salt of claim 13, characterized by an X-ray powder diffraction (XRPD) pattern comprising peaks at 17.7, 18.1, and 22.1±0.2° 2θ using Cu Kα radiation.

15. The salt of claim 14, further characterized by XRPD pattern peaks at 11.0, 16.1, 18.2, 19.8, and 22.9±0.2° 2θ using Cu Kα radiation.

16. The salt of claim 15, further characterized by XRPD pattern peaks at 10.2, 16.5, 16.8, 21.5, 22.2, and 24.3±0.2° 2θ using Cu Kα radiation.

17. The salt of any one of claims 13 to 16, having an XRPD pattern substantially as shown in FIG. 3.

18. The salt of any one of claims 13 to 17, having an endothermic transition at 156° C. to 176° C., as measured by differential scanning calorimetry.

19. The salt of claim 18, wherein the endothermic transition is at 166° C.±3° C.

20. The salt of any one of claims 13 to 19, having a thermogravimetric analysis (“TGA”) substantially as shown in FIG. 4.

21. The salt of claim 1 or 2, wherein X is tartrate.

22. The salt of claim 21, in a crystalline form.

23. The salt of claim 22, characterized by an X-ray powder diffraction (XRPD) pattern comprising peaks at 14.5, 15.6, and 17.5±0.2° 2θ using Cu Kα radiation. [Tartrate Form I]

24. The salt of claim 23, further characterized by XRPD pattern peaks at 18.6, 20.4, 22.8, 24.0, and 24.7±0.2° 2θ using Cu Kα radiation.

25. The salt of claim 24, further characterized by XRPD pattern peaks at 3.1, 3.9, 5.2, 11.3, 14.0, 19.6, 20.9, 22.5, 26.2, and 31.2±0.2° 2θ using Cu Kα radiation.

26. The salt of any one of claims 22 to 25, having an XRPD pattern substantially as shown in FIG. 9.

27. The salt of any one of claims 22 to 26, having an endothermic transition at 200° C. to 210° C., as measured by differential scanning calorimetry.

28. The salt of claim 27, wherein the endothermic transition is at 204° C.±3° C.

29. The salt of any one of claims 22 to 28, having a thermogravimetric analysis (“TGA”) substantially as shown in FIG. 10.

30. The salt of claim 22, characterized by an X-ray powder diffraction (XRPD) pattern comprising peaks at 14.7, 18.9, and 20.8±0.2° 2θ using Cu Kα radiation. [Tartrate Form II]

31. The salt of claim 30, further characterized by XRPD pattern peaks at 11.6, 20.2, 29.8, 29.3, and 35.9±0.2° 2θ using Cu Kα radiation.

32. The salt of claim 31, further characterized by XRPD pattern peaks at 10.4, 13.5, 14.3, 16.9, 18.7, 19.2, 19.6, 20.9, 21.2, 21.7, 23.7, 23.8, 25.1, 26.4, 27.8, 32.0, 33.5, 35.4, 36.7, and 37.5±0.2° 2θ using Cu Kα radiation.

33. The salt of any one of claims 22 and 30 to 32, having an XRPD pattern substantially as shown in FIG. 11.

34. The salt of any one of claims 22 and 30 to 33, having an endothermic transition at 145° C. to 155° C. and 185° C. to 195° C., as measured by differential scanning calorimetry.

35. The salt of claim 34, wherein the endothermic transition is at 148° C. and 188±3° C.

36. The salt of any one of claims 22 and 30 to 35, having a thermogravimetric analysis (“TGA”) substantially as shown in FIG. 12.

37. The salt of claim 1 or 2, wherein the X is malate.

38. The salt of claim 37, in a crystalline form.

39. The salt of claim 38, characterized by an X-ray powder diffraction (XRPD) pattern comprising peaks at 16.9, 18.3, and 23.0±0.2° 2θ using Cu Kα radiation.

40. The salt of claim 39, further characterized by XRPD pattern peaks at 13.8, 17.6, 19.2, 19.8, and 27.6±0.2° 2θ using Cu Kα radiation.

41. The salt of claim 40, further characterized by XRPD pattern peaks at 10.8, 11.8, 14.3, 15.9, 21.3, 21.7, 24.9, 26.7, 27.9, 28.2, and 28.7±0.2° 2θ using Cu Kα radiation.

42. The salt of any one of claims 38 to 41, having an XRPD pattern substantially as shown in FIG. 13.

43. The salt of any one of claims 38 to 42, having an endothermic transition at 135° C. to 145° C., as measured by differential scanning calorimetry.

44. The salt of claim 43, wherein the endothermic transition is at 138° C.±3° C.

45. The salt of any one of claims 38 to 44, having a thermogravimetric analysis (“TGA”) substantially as shown in FIG. 14.

46. The salt of claim 1 or 2, wherein X is glutamate.

47. The salt of claim 46, in a crystalline form.

48. The salt of claim 47, characterized by an X-ray powder diffraction (XRPD) pattern comprising peaks at 21.5, 22.1, and 25.7±0.2° 2θ using Cu Kα radiation.

49. The salt of claim 48, further characterized by XRPD pattern peaks at 20.1, 20.6, 23.9, 26.2, and 30.1±0.2° 2θ using Cu Kα radiation.

50. The salt of claim 49, further characterized by XRPD pattern peaks at 10.3, 13.8, 18.0, 23.2, 27.7, 31.5, 33.8, 34.9, 35.8, and 38.1±0.2° 2θ using Cu Kα radiation.

51. The salt of any one of claims 47 to 50, having an XRPD pattern substantially as shown in FIG. 15.

52. The salt of any one of claims 47 to 51, having an endothermic transition at 95° C. to 105° C. and 198° C. to 208° C., as measured by differential scanning calorimetry.

53. The salt of claim 52, wherein the endothermic transition is at 99° C. and 203° C.±3° C.

54. The salt of any one of claims 47 to 53, having a thermogravimetric analysis (“TGA”) substantially as shown in FIG. 16.

55. The salt of claim 1 or 2, wherein X is adipate.

56. The salt of claim 55, in a crystalline form.

57. The salt of claim 56, characterized by an X-ray powder diffraction (XRPD) pattern comprising peaks at 14.8, 17.7, and 21.6±0.2° 2θ using Cu Kα radiation.

58. The salt of claim 57, further characterized by XRPD pattern peaks at 13.0, 17.4, 19.3, 23.9, 24.8, and 25.9±0.2° 2θ using Cu Kα radiation.

59. The salt of claim 58, further characterized by XRPD pattern peaks at 14.2, 18.7, 23.7, 25.3, and 25.4±0.2° 2θ using Cu Kα radiation.

60. The salt of any one of claims 56 to 59, having an XRPD pattern substantially as shown in FIG. 17.

61. The salt of any one of claims 56 to 60, having an endothermic transition at 128° C. to 138° C., as measured by differential scanning calorimetry.

62. The salt of claim 61, wherein the endothermic transition is at 132° C.±3° C.

63. The salt of any one of claims 56 to 62, having a thermogravimetric analysis (“TGA”) substantially as shown in FIG. 18.

64. The salt of claim 1 or 2, wherein X is fumarate and the salt is a hemi-fumarate.

65. A process for synthesizing (S)-mepazine, or a salt thereof: 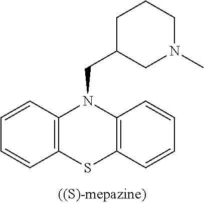
comprising:
(a) admixing compound (J), a base, and a leaving group reagent in a solvent to form compound (K): 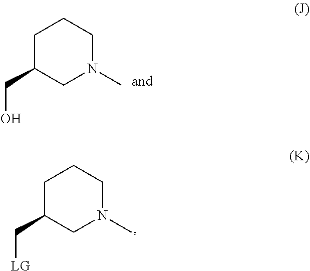
wherein LG is a leaving group;
(b) forming a hydrochloride salt of compound (K); and
(c) admixing the hydrochloride salt of compound (K) and phenothiazine in a solvent to form (S)-mepazine.

66. The process of claim 65 or 66, further comprising step (d): admixing (S)-mepazine with an organic diacid to form a salt of structure 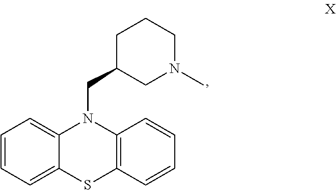 [SMILES: CN1CCC[C@H](CN2c3ccccc3Sc3ccccc32)C1]
wherein X comprises a conjugate base of the organic diacid.

67. The process of claim 66, wherein the salt formed in step (d) is the salt of any one of claims 1 to 64.

68. The process of any one of claims 65 to 69, wherein compound (J) is prepared by admixing compound (I) with LiAlH₄:

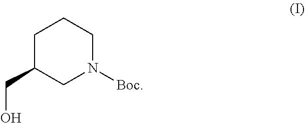 [SMILES: CC(C)(C)OC(=O)N1CCC[C@H](CO)C1]

69. The process of any one of claims 65 to 68, wherein the base of step (a) comprises an amine base.

70. The process of claim 69, wherein the amine base comprises triethyl amine, trimethyl amine, pyridine, 4,-dimethylaminopyridine, aniline, diisopropylamine, 1,8-diazabicyclo [5.4.0] undec-7-ene (DBU), 1,4-diazabicyclo[2.2.2] octane (DABCO), 2,6-lutidine, or a combination thereof.

71. The process of any one of claims 65 to 70, wherein the leaving group reagent comprises mesyl chloride, tosyl chloride, nosyl chloride, methanesulfonic anhydride, para-toluenesulfonic anhydride, or a combination thereof.

72. The process of claim 71, wherein the leaving group reagent is tosyl chloride.

73. The process of any one of claims 65 to 72, wherein the admixing of step (a) occurs at a temperature of −10° ° C. to 25° C.

74. The process of any one of claims 65 to 73, wherein the admixing of step (a) occurs for 1 hour to 36 hours.

75. The process of claim 74, wherein the admixing of step (a) occurs for 15 hours to 25 hours.

76. The process of any one of claims 65 to 75, wherein the solvent of step (a) comprises 2-methyl tetrahydrofuran, tetrahydrofuran, 1,4-dioxane, diethyl ether, dibutyl ether, methyl tert-butyl ether, diisopropyl ether or a combination thereof.

77. The process of any one of claims 65 to 77, wherein step (b) further comprises filtering the hydrochloride salt of compound (K).

78. The process of claim 77, further comprising isolating the crystalline hydrochloride salt of compound (K).

79. The process of any one of claims 65 to 78, wherein the solvent of step (c) comprises a polar aprotic solvent.

80. The process of claim 79, wherein the polar aprotic solvent comprises dimethyl formamide, dimethyl acetamide, N-methyl-2-pyrrolidone, or a combination thereof.

81. The process of claim 80, wherein the polar aprotic solvent comprises N-methyl-2-pyrrolidone.

82. The process of any one of claims 65 to 81, wherein the admixing of step (c) occurs for 1 hour to 36 hours.

83. The process of claim 82, wherein the admixing of step (c) occurs for 15 hours to 20 hours.

84. The process of any one of claims 65 to 83, wherein step (c) further comprises a base.

85. The process of claim 84, wherein the base comprises lithium hydride, sodium hydride, potassium hydride, or a combination thereof.

86. The process of claim 85, wherein the base is sodium hydride.

87. A pharmaceutical formulation comprising (S)-mepazine or a pharmaceutically acceptable salt thereof, and an excipient, in the form of a tablet.

88. The pharmaceutical formulation of claim 87, wherein the tablet is an immediate release tablet.

89. The pharmaceutical formulation of claim 88, wherein at least 90% of the (S)-mepazine or salt thereof is released or dissolved within 6 hours, optionally at least 90% of the (S)-mepazine or salt thereof is released or dissolved within 2 hours, optionally, at least 85% of the(S)-mepazine or salt thereof is released or dissolved within 45 minutes.

90. The pharmaceutical formulation of claim 87, 88, or 89, wherein the (S)-mepazine is present as a salt of any one of claims 1 to 64.

91. The pharmaceutical formulation of claim 90, wherein the (S)-mepazine is present as a salt of any one of claims 3 to 11.

92. The pharmaceutical formulation of any one of claims 87 to 91 wherein the (S)-mepazine or salt thereof is present in an amount of 10% w/w to 50% w/w in the formulation.

93. The pharmaceutical formulation of claim 92, wherein the (S)-mepazine or salt thereof is present in an amount of 20% w/w to 30% w/w in the formulation.

94. The pharmaceutical formulation of any one of claims 87 to 93, wherein the excipient comprises a filler, a lubricant, a disintegrant, a binder, an anti-tacking agent, a flow aid, a wetting agent, or a combination thereof.

95. The pharmaceutical formulation of claim 94, wherein the excipient comprises lactose, cellulose, microcrystalline cellulose, dibasic calcium phosphate, mannitol, croscarmellose sodium, sodium starch glycolate, hydroxyl propyl cellulose, magnesium stearate, colloidal silicon dioxide, sodium stearyl fumarate, hydroxyl propyl methyl cellulose (HPMC), polyethylene oxide, talc, or a combination thereof.

96. The pharmaceutical formulation of claim 95, wherein the excipient comprises lactose, microcrystalline cellulose, croscarmellose sodium, colloidal silicon dioxide, hydroxyl propyl cellulose, and magnesium stearate.

97. The pharmaceutical formulation of any one of claims 87 to 96, wherein the excipient is present in an amount of about 50% w/w to about 90% w/w.

98. The pharmaceutical formulation of claim 97, wherein the excipient is present in an amount of about 70% w/w to about 80% w/w.

99. The pharmaceutical formulation of any one of claims 87 to 98, wherein the excipient does not include a sodium lauryl sulfate.

100. The pharmaceutical formulation of any one of claims 87 to 99, wherein the formulation comprises at least 99% of the (S)-mepazine or salt thereof, upon storage at 40° C.±2° C. and 75% relative humidity (RH)±5% RH in an open container for 4 weeks.

101. The pharmaceutical formulation of claim 100, wherein the formulation comprises at least 99.9% of the (S)-mepazine or salt thereof, upon storage at 40° C.±2° C. and 75% relative humidity (RH)±5% RH in an open container for 4 weeks.

102. The pharmaceutical formulation of any one of claims 87 to 101, further comprising up to 0.5 wt % of (S)-mepazine sulfoxide.

103. A method of treating a subject suffering from cancer, comprising administering to the subject a therapeutically effective amount of the salt of any one of claims 1-64 or the pharmaceutical formulation of any one of claims 87 to 102.

104. The method of claim 103, wherein the cancer is a carcinoma, a melanoma, a sarcoma, a myeloma, a leukemia, or a lymphoma.

105. The method of claim 103, wherein the cancer is a melanoma, colon cancer ovarian cancer, prostate cancer or cervical cancer.

106. The method of claim 103, wherein cancer is a solid tumor.

107. The method of claim 106, wherein the solid tumor is an Adrenocortical Tumor, an Alveolar Soft Part Sarcoma, a Chondrosarcoma, a Colorectal Carcinoma, a Desmoid Tumors, a Desmoplastic Small Round Cell Tumor, an Endocrine Tumors, an Endodermal Sinus Tumor, an Epithelioid Hemangioendothelioma, a Ewing Sarcoma, a Germ Cell Tumors (Solid Tumor), a Giant Cell Tumor of Bone and Soft Tissue, a Hepatoblastoma, a Hepatocellular Carcinoma, a Melanoma, a Nephroma, a Neuroblastoma, a Non-Rhabdomyosarcoma Soft Tissue Sarcoma (NRSTS), an Osteosarcoma, a Paraspinal Sarcoma, a Renal Cell Carcinoma, a Retinoblastoma, a Rhabdomyosarcoma, a Synovial Sarcoma, or a Wilms Tumor.

108. A method of treating a subject suffering from an autoimmune disease, comprising administering to the subject a therapeutically effective amount of the salt of any one of claims 1-64 or the pharmaceutical formulation of any one of claims 87 to 102.

109. The method of claim 103, wherein the autoimmune disease is multiple sclerosis.

110. A free base crystalline form of (S)-mepazine 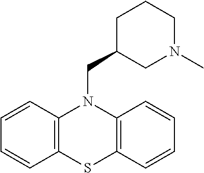 [SMILES: CN1CCC[C@H](CN2c3ccccc3Sc3ccccc32)C1]
(S)-mepazine)).

111. The free base crystalline form of (S)-mepazine of claim 110, having an XRPD pattern substantially as shown in FIG. 19.
